SEQUENCE LISTING (163 sequences)

SEQ ID NO: 1 - protein, 466 aa, Homo sapiens
MEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAVDVVLDPDTAHPDLFLSEDRRSVRRCPFRHLGESVPDNPERFDSQPCVLGRESFASGKHYWEVEVENVIEWTVGVCRDSVERKGEVLLIPQNGFWTLEMHKGQYRAVSSPDRILPLKESLCRVGVFLDYEAGDVSFYNMRDRSHIYTCPRSAFSVPVRPFFRLGCEDSPIFICPALTGANGVTVPEEGLTLHRVGTHQSL

SEQ ID NO: 2 - protein, 330 aa, Homo sapiens
MESAAALHFSRPASLLLLLLSLCALVSAQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAGPV

SEQ ID NO: 3 - protein, 527 aa, Homo sapiens
MESAAALHFSRPASLLLLLLSLCALVSAQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAVDVVLDPDTAHPDLFLSEDRRSVRRCPFRHLGESVPDNPERFDSQPCVLGRESFASGKHYWEVEVENVIEWTVGVCRDSVERKGEVLLIPQNGFWTLEMHKGQYRAVSSPDRILPLKESLCRVGVFLDYEAGDVSFYNMRDRSHIYTCPRSAFSVPVRPFFRLGCEDSPIFICPALTGANGVTVPEEGLTLHRVGTHQSL

SEQ ID NO: 4 - protein, 330 aa, Homo sapiens; identical to SEQ ID NO: 2

SEQ ID NO: 5 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 6 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 7 - protein, 318 aa, Artificial Sequence
  Lactadherin C1C2 sequence (aka MFG-E8)
CVEPLGMENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 8 - protein, 9 aa, Homo sapiens
CLSYYPSYC

SEQ ID NO: 9 - DNA, 19 bp, Artificial Sequence
  primer
tgggcaggagtcatgtcag

SEQ ID NO: 10 - DNA, 24 bp, Artificial Sequence
  primer
gcaggatcaaactctgttatcttc

SEQ ID NO: 11 - DNA, 21 bp, Artificial Sequence
  primer
ggctctgtgtgtatatggtgc

SEQ ID NO: 12 - DNA, 24 bp, Artificial Sequence
  primer
ctgacgatacatctgtgttctttg

SEQ ID NO: 13 - DNA, 26 bp, Artificial Sequence
  primer
ataccggtgccattgagttggtgcct

SEQ ID NO: 14 - DNA, 33 bp, Artificial Sequence
  primer
tgttccggatatccttggggtagaattccttca

SEQ ID NO: 15 - DNA, 27 bp, Artificial Sequence
  primer
ataccggtgcaggtcacctagagcaac

SEQ ID NO: 16 - DNA, 32 bp, Artificial Sequence
  primer
cagcaattgaaggaagaaaaatagtgggcttg

SEQ ID NO: 17 - DNA, 21 bp, Artificial Sequence
  primer
atgaagggcgcagccatcggc

SEQ ID NO: 18 - DNA, 18 bp, Artificial Sequence
  primer
gctacaccgcagtgtggc

SEQ ID NO: 19 - DNA, 30 bp, Artificial Sequence
  primer
cttcatctacgcagagaaggacatctacgg

SEQ ID NO: 20 - DNA, 18 bp, Artificial Sequence
  primer
gtcatggtgttgccctgg

SEQ ID NO: 21 - DNA, 28 bp, Artificial Sequence
  primer
ctgtgacacagctggaatgggcggcgag

SEQ ID NO: 22 - DNA, 18 bp, Artificial Sequence
  primer
gcgcagtagtagctgccc

SEQ ID NO: 23 - DNA, 21 bp, Artificial Sequence
  primer
ggacatctggcacagccccag

SEQ ID NO: 24 - DNA, 18 bp, Artificial Sequence
  primer
agcgccatacacacacag

SEQ ID NO: 25 - DNA, 22 bp, Artificial Sequence
  primer
caagaccgccgcactggaatgc

SEQ ID NO: 26 - DNA, 18 bp, Artificial Sequence
  primer
ctcagtgtcttggtgctg

SEQ ID NO: 27 - DNA, 21 bp, Artificial Sequence
  primer
gccagactggcatgcgtggtg

SEQ ID NO: 28 - DNA, 20 bp, Artificial Sequence
  primer
ggtcttgctcagtgtcttgg

SEQ ID NO: 29 - DNA, 21 bp, Artificial Sequence
  primer
gtccggcatcgcaatcagcgc

SEQ ID NO: 30 - DNA, 19 bp, Artificial Sequence
  primer
accacgcattccagtctgg

SEQ ID NO: 31 - DNA, 22 bp, Artificial Sequence
  primer
gtccatcagcgccgatggcacc

SEQ ID NO: 32 - DNA, 20 bp, Artificial Sequence
  primer
accaggaactggatcacttc

SEQ ID NO: 33 - DNA, 21 bp, Artificial Sequence
  primer
ctacgatggcgccgtgcggaa

SEQ ID NO: 34 - DNA, 21 bp, Artificial Sequence
  primer
ctgatggacaccaggaactgg

SEQ ID NO: 35 - DNA, 22 bp, Artificial Sequence
  primer
caccgtgcgggcagagagcggc

SEQ ID NO: 36 - DNA, 20 bp, Artificial Sequence
  primer
ccatcgtagctgatggacac

SEQ ID NO: 37 - DNA, 24 bp, Artificial Sequence
  primer
gcggaaagaggccggcatcccttc

SEQ ID NO: 38 - DNA, 18 bp, Artificial Sequence
  primer
acggtgccatcgtagctg

SEQ ID NO: 39 - DNA, 21 bp, Artificial Sequence
  primer
cggcatccctgctggcaagtt

SEQ ID NO: 40 - DNA, 18 bp, Artificial Sequence
  primer
ctctctttccgcacggtg

SEQ ID NO: 41 - DNA, 24 bp, Artificial Sequence
  primer
ggcaagttcgcggtggacagaatc

SEQ ID NO: 42 - DNA, 18 bp, Artificial Sequence
  primer
agaagggatgccgctctc

SEQ ID NO: 43 - DNA, 21 bp, Artificial Sequence
  primer
agaatccccgcgacaagcacc

SEQ ID NO: 44 - DNA, 18 bp, Artificial Sequence
  primer
gtccacctcgaacttgcc

SEQ ID NO: 45 - DNA, 28 bp, Artificial Sequence
  primer
actgaccatcgccaacgtggaaaagcag

SEQ ID NO: 46 - DNA, 18 bp, Artificial Sequence
  primer
gtgctggtgcttgtctcg

SEQ ID NO: 47 - DNA, 25 bp, Artificial Sequence
  primer
gaccatccacgccgtggaaaagcag

SEQ ID NO: 48 - DNA, 18 bp, Artificial Sequence
  primer
agtgtgctggtgcttgtc

SEQ ID NO: 49 - DNA, 24 bp, Artificial Sequence
  primer
cacaacgtggcaaagcaggatatc

SEQ ID NO: 50 - DNA, 18 bp, Artificial Sequence
  primer
gatggtcagtgtgctggt

SEQ ID NO: 51 - DNA, 22 bp, Artificial Sequence
  primer
cgtggaaaaggcggatatcgcc

SEQ ID NO: 52 - DNA, 18 bp, Artificial Sequence
  primer
ttgtggatggtcagtgtg

SEQ ID NO: 53 - DNA, 29 bp, Artificial Sequence
  primer
agagctgggcgcgaaaatcaaggtgttcg

SEQ ID NO: 54 - DNA, 18 bp, Artificial Sequence
  primer
tgttgggcttcccacagg

SEQ ID NO: 55 - DNA, 25 bp, Artificial Sequence
  CRISPR 2 top
tcacaaaggtggttcttcctgtttt

SEQ ID NO: 56 - DNA, 25 bp, Artificial Sequence
  CRISPR 2 bottom
aggaagaaccacctttgtgacggtg

SEQ ID NO: 57 - DNA, 20 bp, Artificial Sequence
  CRISPR 4 top
caatagatgcatacggcaat

SEQ ID NO: 58 - DNA, 25 bp, Artificial Sequence
  CRISPR 4 bottom
attgccgtatgcatctattgcggtg

SEQ ID NO: 59 - DNA, 20 bp, Artificial Sequence
  sc-404202 A
ggcacttacgagatgcatac

SEQ ID NO: 60 - DNA, 20 bp, Artificial Sequence
  sc-404202 B
gagagacattcagcctataa

SEQ ID NO: 61 - DNA, 20 bp, Artificial Sequence
  sc-404202 C
accatcagaagttccctcct

SEQ ID NO: 62 - DNA, 42 bp, Artificial Sequence
  2A1 CRISPR1 MiSeqF
gtgacctatgaactcaggagtccttgagtgacgggagaggtt

SEQ ID NO: 63 - DNA, 41 bp, Artificial Sequence
  2A1 CRISPR1 MiSeqR
ctgagacttgcacatcgcagctccttttggacagtgctggt

SEQ ID NO: 64 - DNA, 42 bp, Artificial Sequence
  2A1 CRISPR2 MiSeqF
gtgacctatgaactcaggagtccctttgttgaacagcccagt

SEQ ID NO: 65 - DNA, 41 bp, Artificial Sequence
  2A1 CRISPR2 MiSeqR
ctgagacttgcacatcgcagctaggacctgccttcttggaa

SEQ ID NO: 66 - DNA, 42 bp, Artificial Sequence
  2A1 CRISPR3 MiSeqF
gtgacctatgaactcaggagtcccgagaaaaatgctgaggac

SEQ ID NO: 67 - DNA, 41 bp, Artificial Sequence
  2A1 CRISPR3 MiSeqR
ctgagacttgcacatcgcagcaatgggcctgaggttaggag

SEQ ID NO: 68 - DNA, 42 bp, Artificial Sequence
  2A1 CRISPR4 MiSeqF
gtgacctatgaactcaggagtcagaaagcaggagagcaggtg

SEQ ID NO: 69 - DNA, 41 bp, Artificial Sequence
  2A1 CRISPR4 MiSeqR
ctgagacttgcacatcgcagcttgcacacgttctttctcca

SEQ ID NO: 70 - protein, 311 aa, Artificial Sequence
  Soluble delta chain 2
  X (20)..(20): V or I
  X (49)..(49): M or I
ETGAIELVPEHQTVPVSIGXPATLRCSMKGEAIGNYYINWYRKTQGNTXTFIYREKDIYGPGFKDNFQGDIDIAKNLAVLKILAPSERDEGSYYCACDPLLGSERLGDTGIDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSASGLVPRGSGSGLTDTLQAETDQLEDKKSALQTEIANLLKEKEKLEFILAAYGSGLNDIFEAQKIEWHEHHHHHH

SEQ ID NO: 71 - protein, 308 aa, Artificial Sequence
  Soluble delta chain 2 (clone 3)
  X (20)..(20): V or I
  X (49)..(49): M or I
ETGAIELVPEHQTVPVSIGXPATLRCSMKGEAIGNYYINWYRKTQGNTXTFIYREKDIYGPGFKDNFQGDIDIAKNLAVLKILAPSERDEGSYYCACDPVQVTGGYKVDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSASGLVPRGSGSGLTDTLQAETDQLEDKKSALQTEIANLLKEKEKLEFILAAYGSGLNDIFEAQKIEWHEHHHHHH

SEQ ID NO: 72 - protein, 308 aa, Artificial Sequence
  Soluble delta chain 2 (clone 4)
  X (20)..(20): V or I
  X (49)..(49): M or I
ETGAIELVPEHQTVPVSIGXPATLRCSMKGEAIGNYYINWYRKTQGNTXTFIYREKDIYGPGFKDNFQGDIDIAKNLAVLKILAPSERDEGSYYCACDTVQRLGDRPTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSASGLVPRGSGSGLTDTLQAETDQLEDKKSALQTEIANLLKEKEKLEFILAAYGSGLNDIFEAQKIEWHEHHHHHH

SEQ ID NO: 73 - protein, 306 aa, Artificial Sequence
  Soluble delta chain 2 (clone 5)
  X (20)..(20): V or I
  X (49)..(49): M or I
ETGAIELVPEHQTVPVSIGXPATLRCSMKGEAIGNYYINWYRKTQGNTXTFIYREKDIYGPGFKDNFQGDIDIAKNLAVLKILAPSERDEGSYYCACDGILGDSHTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSASGLVPRGSGSGLTDTLQAETDQLEDKKSALQTEIANLLKEKEKLEFILAAYGSGLNDIFEAQKIEWHEHHHHHH

SEQ ID NO: 74 - protein, 307 aa, Artificial Sequence
  Soluble delta chain 2 (clone 7)
  X (20)..(20): V or I
  X (49)..(49): M or I
ETGAIELVPEHQTVPVSIGXPATLRCSMKGEAIGNYYINWYRKTQGNTXTFIYREKDIYGPGFKDNFQGDIDIAKNLAVLKILAPSERDEGSYYCACDRVLGDTRWTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSASGLVPRGSGSGLTDTLQAETDQLEDKKSALQTEIANLLKEKEKLEFILAAYGSGLNDIFEAQKIEWHEHHHHHH

SEQ ID NO: 75 - protein, 123 aa, Artificial Sequence
  soluble delta chain 2 variable region
  X (17)..(17): V or I
  X (46)..(46): V or I
AIELVPEHQTVPVSIGXPATLRCSMKGEAIGNYYINWYRKTQGNTXTFIYREKDIYGPGFKDNFQGDIDIAKNLAVLKILAPSERDEGSYYCACDPLLGSERLGDTGIDKLIFGKGTRVTVEP

SEQ ID NO: 76 - protein, 120 aa, Artificial Sequence
  variable region of gamma 2 (clone 3)
  X (17)..(17): V or I
  X (46)..(46): M or I
AIELVPEHQTVPVSIGXPATLRCSMKGEAIGNYYINWYRKTQGNTXTFIYREKDIYGPGFKDNFQGDIDIAKNLAVLKILAPSERDEGSYYCACDPVQVTGGYKVDKLIFGKGTRVTVEP

SEQ ID NO: 77 - protein, 120 aa, Artificial Sequence
  variable region of Soluble delta chain 2 (clone 4)
  X (17)..(17): V ot I
  X (46)..(46): M ot I
AIELVPEHQTVPVSIGXPATLRCSMKGEAIGNYYINWYRKTQGNTXTFIYREKDIYGPGFKDNFQGDIDIAKNLAVLKILAPSERDEGSYYCACDTVQRLGDRPTDKLIFGKGTRVTVEP

SEQ ID NO: 78 - protein, 118 aa, Artificial Sequence
  variable region of Soluble delta chain 2 (clone 5)
  X (17)..(17): V or I
  X (46)..(46): M or I
AIELVPEHQTVPVSIGXPATLRCSMKGEAIGNYYINWYRKTQGNTXTFIYREKDIYGPGFKDNFQGDIDIAKNLAVLKILAPSERDEGSYYCACDGILGDSHTDKLIFGKGTRVTVEP

SEQ ID NO: 79 - protein, 119 aa, Artificial Sequence
  variable region of Soluble delta chain 2 (clone 7)
  X (17)..(17): V or I
  X (46)..(46): M or I
AIELVPEHQTVPVSIGXPATLRCSMKGEAIGNYYINWYRKTQGNTXTFIYREKDIYGPGFKDNFQGDIDIAKNLAVLKILAPSERDEGSYYCACDRVLGDTRWTDKLIFGKGTRVTVEP

SEQ ID NO: 80 - protein, 19 aa, Artificial Sequence
  CDR3 gamma (clone 3)
CACDPVQVTGGYKVDKLIF

SEQ ID NO: 81 - protein, 19 aa, Artificial Sequence
  of CDR3 gamma (clone 4)
CACDTVQRLGDRPTDKLIF

SEQ ID NO: 82 - protein, 17 aa, Artificial Sequence
  of CDR3 gamma (clone 5)
CACDGILGDSHTDKLIF

SEQ ID NO: 83 - protein, 22 aa, Artificial Sequence
  of CDR3 gamma (clone 6)
CACDPLLGSERLGDTGIDKLIF

SEQ ID NO: 84 - protein, 18 aa, Artificial Sequence
  of CDR3 gamma (clone 7)
CACDRVLGDTRWTDKLIF

SEQ ID NO: 85 - protein, 296 aa, Artificial Sequence
  Soluble gamma chain 9
ETGAGHLEQPQISSTKTLSKTARLECVVSGITISATSVYWYRERPGEVIQFLVSISYDGTVRKESGIPSGKFEVDRIPETSTSTLTIHNVEKQDIATYYCALWESQELGKKIKVFGPGTKLIITDKQLDADVSPKPTIFLPSIAETKLQKAGTYLCLLEKFFPDVIKIHWQEKKSNTILGSQEGNTMKTNDTYMKFSWLTVPEESLDKEHRCIVRHENNKNGVDQEIIFPPIKTDVITMDPKDNASGLVPRGSGSGRIARLEEKVKTLKAQNSELASTANMLREQVAQLKQKVMNY

SEQ ID NO: 86 - protein, 296 aa, artificial
  Soluble gamma chain 9 (clone 3)
ETGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEVHELGKKIKVFGPGTKLIITDKQLDADVSPKPTIFLPSIAETKLQKAGTYLCLLEKFFPDVIKIHWQEKKSNTILGSQEGNTMKTNDTYMKFSWLTVPEESLDKEHRCIVRHENNKNGVDQEIIFPPIKTDVITMDPKDNASGLVPRGSGSGRIARLEEKVKTLKAQNSELASTANMLREQVAQLKQKVMNY

SEQ ID NO: 87 - protein, 296 aa, artificial
  gamma 9 (clone 4)
ETGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEVQELGKKIKVFGPGTKLIITDKQLDADVSPKPTIFLPSIAETKLQKAGTYLCLLEKFFPDVIKIHWQEKKSNTILGSQEGNTMKTNDTYMKFSWLTVPEESLDKEHRCIVRHENNKNGVDQEIIFPPIKTDVITMDPKDNASGLVPRGSGSGRIARLEEKVKTLKAQNSELASTANMLREQVAQLKQKVMNY

SEQ ID NO: 88 - protein, 296 aa, Artificial Sequence
  gamma 9 (clone 5)
ETGAGHLEQPQISSTKTLSKTARLECVVSGITISATSVYWYRERPGEVIQFLVSISYDGTVRKESGIPSGKFEVDRIPETSTSTLTIHNVEKQDIATYYCALWELAELGKKIKVFGPGTKLIITDKQLDADVSPKPTIFLPSIAETKLQKAGTYLCLLEKFFPDVIKIHWQEKKSNTILGSQEGNTMKTNDTYMKFSWLTVPEESLDKEHRCIVRHENNKNGVDQEIIFPPIKTDVITMDPKDNASGLVPRGSGSGRIARLEEKVKTLKAQNSELASTANMLREQVAQLKQKVMNY

SEQ ID NO: 89 - protein, 296 aa, Artificial Sequence; identical to SEQ ID NO: 86
  gamma 9 (clone 7)

SEQ ID NO: 90 - protein, 121 aa, Artificial Sequence
  soluble gamma chain 9 variable region
AGHLEQPQISSTKTLSKTARLECVVSGITISATSVYWYRERPGEVIQFLVSISYDGTVRKESGIPSGKFEVDRIPETSTSTLTIHNVEKQDIATYYCALWESQELGKKIKVFGPGTKLIIT

SEQ ID NO: 91 - protein, 121 aa, Artificial Sequence
  variable region of gamma 9 (clone 3)
AGHLEQPQISSTKTLSKTARLECVVSGITISATSVYWYRERPGEVIQFLVSISYDGTVRKESGIPSGKFEVDRIPETSTSTLTIHNVEKQDIATYYCALWEVHELGKKIKVFGPGTKLIIT

SEQ ID NO: 92 - protein, 121 aa, Artificial Sequence
  variable region of gamma 9 (clone 4)
AGHLEQPQISSTKTLSKTARLECVVSGITISATSVYWYRERPGEVIQFLVSISYDGTVRKESGIPSGKFEVDRIPETSTSTLTIHNVEKQDIATYYCALWEVQELGKKIKVFGPGTKLIIT

SEQ ID NO: 93 - protein, 121 aa, Artificial Sequence
  variable region of gamma 9 (clone 5)
AGHLEQPQISSTKTLSKTARLECVVSGITISATSVYWYRERPGEVIQFLVSISYDGTVRKESGIPSGKFEVDRIPETSTSTLTIHNVEKQDIATYYCALWELAELGKKIKVFGPGTKLIIT

SEQ ID NO: 94 - protein, 121 aa, Artificial Sequence; identical to SEQ ID NO: 91
  variable region of gamma 9 (clone 7)

SEQ ID NO: 95 - protein, 16 aa, Artificial Sequence
  CDR3 gamma (clone 3)
CALWEVHELGKKIKVF

SEQ ID NO: 96 - protein, 16 aa, Artificial Sequence
  CDR3 gamma (clone 4)
CALWEVQELGKKIKVF

SEQ ID NO: 97 - protein, 16 aa, Artificial Sequence
  CDR3 gamma (clone 5)
CALWELAELGKKIKVF

SEQ ID NO: 98 - protein, 16 aa, Artificial Sequence
  CDR3 gamma (clone 6)
CALWESQELGKKIKVF

SEQ ID NO: 99 - protein, 16 aa, Artificial Sequence
  CDR3 gamma (clone 7)
CALWEVHELGKKIKVF

SEQ ID NO: 100 - protein, 116 aa, Artificial Sequence
  amino acid sequence of Hu34C VH
EVQLLESGGGLVQPGGSLRLSCAASGFTFSHYPMIWVRQAPGKGLEWVSSISSSGGSTHYADSVKGRFTISRDNSKNTLYLQMNSLRAEDTAMYYCAALVSDTDFWGQGTLVTVSS

SEQ ID NO: 101 - protein, 107 aa, Artificial Sequence
  amino acid sequence of Hu34C VL
DIQMTQSPATLSVSPGESTTLSCRASQSVTSNLAWYQQKPGQAPSLLIYGASTRATGIPARFSGSGSGTEFTLTISSLQSEDSAVYYCQQYNNWPRTFGQGTKVEIK

SEQ ID NO: 102 - protein, 8 aa, Artificial Sequence
  amino acid sequence of Hu34C VH CDR1
GFTFSHYP

SEQ ID NO: 103 - protein, 8 aa, Artificial Sequence
  amino acid sequence of Hu34C VH CDR2
ISSSGGST

SEQ ID NO: 104 - protein, 9 aa, Artificial Sequence
  amino acid sequence of Hu34C VH CDR3
AALVSDTDF

SEQ ID NO: 105 - protein, 6 aa, Artificial Sequence
  amino acid sequence of Hu34C VL CDR1
QSVTSN

SEQ ID NO: 106 - protein, 3 aa, Artificial Sequence
  amino acid sequence of Hu34C VL CDR2
GAS

SEQ ID NO: 107 - protein, 9 aa, Artificial Sequence
  amino acid sequence of Hu34C VL CDR3
QQYNNWPRT

SEQ ID NO: 108 - protein, 116 aa, Artificial Sequence
  amino acid sequence of clone 227 VH
EVQLLESGGGLVQPGGSLRLSCAASGFTFSIYSMWWVRQAPGKGLEWVSRISPSGGGTYYADSVKGRFTISRDNSKNTLYLQMNSLRAEDTAVYYCTTPTVTTDYWGQGTLVTVSS

SEQ ID NO: 109 - protein, 108 aa, Artificial Sequence
  amino acid sequence of clone 227 VL
DIQMTQSPGTLSLSPGERATLSCRASQSVSSSYLAWYQQKPGQAPRLLIYGASSRATGIPDRFSGSGSGTDFTLTISRLEPEDFAVYYCQQYDSSLWTFGQGTKVEVK

SEQ ID NO: 110 - protein, 8 aa, Artificial Sequence
  amino acid sequence of clone 227 VH CDR1
GFTFSIYS

SEQ ID NO: 111 - protein, 8 aa, Artificial Sequence
  amino acid sequence of clone 227 VH CDR2
ISPSGGGT

SEQ ID NO: 112 - protein, 9 aa, Artificial Sequence
  amino acid sequence of clone 227 VH CDR3
TTPTVTTDY

SEQ ID NO: 113 - protein, 7 aa, Artificial Sequence
  amino acid sequence of clone 227 VL CDR1
QSVSSSY

SEQ ID NO: 114 - protein, 3 aa, Artificial Sequence
  amino acid sequence of clone 227 VL CDR2
GAS

SEQ ID NO: 115 - protein, 9 aa, Artificial Sequence
  amino acid sequence of clone 227 VL CDR3
QQYDSSLWT

SEQ ID NO: 116 - protein, 115 aa, Artificial Sequence
  amino acid sequence of clone 236 VH
EVQLLESGGGLVQPGGSLRLSCAASGFTFSHYSMSWVRQAPGKGLEWVSGISSSGGFTPYADSVKGRFTISRDNSKNTLYLQMNSLRAEDTAVYYCTGGSGFDIWGQGTMVTVSS

SEQ ID NO: 117 - protein, 107 aa, Artificial Sequence
  amino acid sequence of clone 236 VL
DIQMTQSPSTLSVSVGDRVTITCRASQTISSWLAWFQQKPGQAPKLLIYRASTLETGVPSRFSGSESGTTFTLTINGLQPDDLATYYCQQYATFPWTFGQGTKVEVK

SEQ ID NO: 118 - protein, 8 aa, Artificial Sequence
  amino acid sequence of clone 236 VH CDR1
GFTFSHYS

SEQ ID NO: 119 - protein, 8 aa, Artificial Sequence
  amino acid sequence of clone 236 VH CDR2
ISSSGGFT

SEQ ID NO: 120 - protein, 8 aa, Artificial Sequence
  amino acid sequence of clone 236 VH CDR3
TGGSGFDI

SEQ ID NO: 121 - protein, 6 aa, Artificial Sequence
  amino acid sequence of clone 236 VL CDR1
QTISSW

SEQ ID NO: 122 - protein, 3 aa, Artificial Sequence
  amino acid sequence of clone 236 VL CDR2
RAS

SEQ ID NO: 123 - protein, 9 aa, Artificial Sequence
  amino acid sequence of clone 236 VL CDR3
QQYATFPWT

SEQ ID NO: 124 - protein, 116 aa, Artificial Sequence
  amino acid sequence of clone 266 VH
EVQLLESGGGLVQPGGSLRLSCAASGFTFSMYTMMWVRQAPGKGLEWVSGIGSSGGHTKYADSVKGRFTISRDNSKNTLYLQMNSLRAEDTAVYYCARYGHTFETWGQGTMVTVSS

SEQ ID NO: 125 - protein, 111 aa, Artificial Sequence
  amino acid sequence of clone 266 VL
QYELTQPPSASGTPGQRVTISCSGSSSNIGSNYVYWYQQLPGTAPKLLIYRNNQRPSGVPDRFSGSKSGTSASLAISGLRSEDEADYYCAAWDDSLSGSYVFGTGTKVTVL

SEQ ID NO: 126 - protein, 8 aa, Artificial Sequence
  amino acid sequence of clone 266 VH CDR1
GFTFSMYT

SEQ ID NO: 127 - protein, 8 aa, Artificial Sequence
  amino acid sequence of clone 266 VH CDR2
IGSSGGHT

SEQ ID NO: 128 - protein, 9 aa, Artificial Sequence
  amino acid sequence of clone 266 VH CDR3
ARYGHTFET

SEQ ID NO: 129 - protein, 8 aa, Artificial Sequence
  amino acid sequence of clone 266 VL CDR1
SSNIGSNY

SEQ ID NO: 130 - protein, 3 aa, Artificial Sequence
  amino acid sequence of clone 266 VL CDR2
RNN

SEQ ID NO: 131 - protein, 12 aa, Artificial Sequence
  amino acid sequence of clone 266 VL CDR3
AAWDDSLSGSYV

SEQ ID NO: 132 - protein, 117 aa, Artificial Sequence
  amino acid sequence of clone 267 VH
EVQLLESGGGLVQPGGSLRLSCAASGFTFSTYVMYWVRQAPGKGLEWVSSISPSGGLTVYVDSVKGRFTISRDNSKNTLYLQMNSLRAEDTAVYYCARVGYSSSDIWGQGTMVTVSS

SEQ ID NO: 133 - protein, 110 aa, Artificial Sequence
  amino acid sequence of clone 267 VL
QYELTQPHSVSESPGKTVTISCTRNGGSIASNAVQWYQQRPGSAPITVIYDDSQRPSGVPDRFSGSIDSSSNTASLTISGLTTEDEADYYCLSFDTTSQIFGGGTKLTVL

SEQ ID NO: 134 - protein, 8 aa, Artificial Sequence
  amino acid sequence of clone 267 VH CDR1
GFTFSTYV

SEQ ID NO: 135 - protein, 8 aa, Artificial Sequence
  amino acid sequence of clone 267 VH CDR2
ISPSGGLT

SEQ ID NO: 136 - protein, 10 aa, Artificial Sequence
  amino acid sequence of clone 267 VH CDR3
ARVGYSSSDI

SEQ ID NO: 137 - protein, 8 aa, Artificial Sequence
  amino acid sequence of clone 267 VL CDR1
GGSIASNA

SEQ ID NO: 138 - protein, 3 aa, Artificial Sequence
  amino acid sequence of clone 267 VL CDR2
DDS

SEQ ID NO: 139 - protein, 9 aa, Artificial Sequence
  amino acid sequence of clone 267 VL CDR3
LSFDTTSQI

SEQ ID NO: 140 - protein, 108 aa, Artificial Sequence
  amino acid sequence of the VL of antibody 244
QSVLTQPHSVSESPGKTVTISCTRSSGRIGSNYVHWYQHLPGRAPKLLIYGNSNRPSGVPDRFSGSKSGTSASLAITGLQADDEADYYCQTYDNNVWVFGGGTKLTVL

SEQ ID NO: 141 - protein, 8 aa, Artificial Sequence
  amino acid sequence of CDR1 of the VL of antibody 244
SGRIGSNY

SEQ ID NO: 142 - protein, 3 aa, Artificial Sequence
  amino acid sequence of CDR2 of the VL of antibody 244
GNS

SEQ ID NO: 143 - protein, 9 aa, Artificial Sequence
  amino acid sequence of CDR3 of the VL of antibody 244
QTYDNNVWV

SEQ ID NO: 144 - protein, 120 aa, Artificial Sequence
  amino acid sequence of the VH of antibody 244
EVQLLESGGGLVQPGGSLRLSCAASGFTFSEYSMSWVRQAPGKGLEWVSRIGPSGGGTVYADSVKGRFTISRDNSKNTLYLQMNSLRAEDTAMYYCARLGRGAVAELDYWGQGTLVTVSS

SEQ ID NO: 145 - protein, 8 aa, Artificial Sequence
  amino acid sequence of CDR1 of the VH of antibody 244
GFTFSEYS

SEQ ID NO: 146 - protein, 8 aa, Artificial Sequence
  amino acid sequence of CDR2 of the VH of antibody 244
IGPSGGGT

SEQ ID NO: 147 - protein, 13 aa, Artificial Sequence
  amino acid sequence of CDR3 of the VH of antibody 244
ARLGRGAVAELDY

SEQ ID NO: 148 - protein, 110 aa, Artificial Sequence
  amino acid sequence of the VL of antibody 253
QSALTQPPSASGTPGQRVTLSCSGSSSNIGSNRVNWYQQLPGTAPKLLIDSNNQRPSGVPDRFSGSKSGTSASLAISGLRSEDEVFYYCAAWDDSVHGPVFGGGTMVTVL

SEQ ID NO: 149 - protein, 8 aa, Artificial Sequence
  amino acid sequence of CDR1 of the VL of antibody 253
SSNIGSNR

SEQ ID NO: 150 - protein, 3 aa, Artificial Sequence
  amino acid sequence of CDR2 of the VL of antibody 253
SNN

SEQ ID NO: 151 - protein, 11 aa, Artificial Sequence
  amino acid sequence of CDR3 of the VL of antibody 253
AAWDDSVHGPV

SEQ ID NO: 152 - protein, 127 aa, Artificial Sequence
  amino acid sequence of the VH of antibody 253
EVQLLESGGGLVQPGGSLRLSCAASGFTFSTYDMYWVRQAPGKGLEWVSYISPSGGLTTYADSVKGRFTISRDNSKNTLYLQMNSLRAEDTAVYYCARDHYDSSGRATNYYYYMDVWGKGTTVTVSS

SEQ ID NO: 153 - protein, 8 aa, Artificial Sequence
  amino acid sequence of CDR1 of the VH of antibody 253
GFTFSTYD

SEQ ID NO: 154 - protein, 8 aa, Artificial Sequence
  amino acid sequence of CDR2 of the VH of antibody 253
ISPSGGLT

SEQ ID NO: 155 - protein, 20 aa, Artificial Sequence
  amino acid sequence of CDR3 of the VH of antibody 253
ARDHYDSSGRATNYYYYMDV

SEQ ID NO: 156 - protein, 110 aa, Artificial Sequence
  amino acid sequence of the VL of antibody 259
QSELTQPPSASETPGQRVTISCSGSSSNIGSNTVSWFQQLPGSAPRLLIYNDHRRPSGVPDRFSGSKSGTSASLVISGLQSQDEADYYCSAWDDSLNGVVFGGGTKLTVL

SEQ ID NO: 157 - protein, 8 aa, Artificial Sequence
  amino acid sequence of CDR1 of the VL of antibody 259
SSNIGSNT

SEQ ID NO: 158 - protein, 3 aa, Artificial Sequence
  amino acid sequence of CDR2 of the VL of antibody 259
NDH

SEQ ID NO: 159 - protein, 11 aa, Artificial Sequence
  amino acid sequence of CDR3 of the VL of antibody 259
SAWDDSLNGVV

SEQ ID NO: 160 - protein, 120 aa, Artificial Sequence
  amino acid sequence of the VH of antibody 259
EVQLLESGGGLVQPGGSLRLSCAASGFTFSSYAMQWVRQAPGKGLEWVSRIGPSGGGTMYADSVKGRFTISRDNSKNTLYLQMNSLRAEDMAVYYCARDLGGLSVLFDYWGQGTLVTVSS

SEQ ID NO: 161 - protein, 8 aa, Artificial Sequence
  amino acid sequence of CDR1 of the VH of antibody 259
GFTFSSYA

SEQ ID NO: 162 - protein, 8 aa, Artificial Sequence
  amino acid sequence of CDR2 of the VH of antibody 259
IGPSGGGT

SEQ ID NO: 163 - protein, 13 aa, Artificial Sequence
  amino acid sequence of CDR3 of the VH of antibody 259
ARDLGGLSVLFDY

(19) United States
(12) Patent Application Publication
(10) Pub. No.: US 2022/0273713 A1
(43) Pub. Date: Sep. 1, 2022

(54) METHOD OF INHIBITING OR ACTIVATING GAMMA DELTA T CELLS

(71) Applicants: The University of Melbourne, Victoria (AU); Olivia Newton-John Cancer Research Institute, Heidelberg, Victoria (AU); CSL INNOVATION PTY LTD, Parkville, Victoria (AU)

(72) Inventors: Andreas BEHREN, Heidelberg, Victoria (AU); Jonathan CEBON, Heidelberg, Victoria (AU); Marc Rigau CORTAL, Victoria (AU); Thomas Samuel FULFORD, Victoria (AU); Dale Ian GODFREY, Victoria (AU); Andrew HAMMET, Parkville, Victoria (AU); Simone OSTROUSKA, Heidelberg, Victoria (AU); Con PANOUSIS, Parkville, Victoria (AU); Adam Peter ULDRICH, Victoria (AU)

(73) Assignees: The University of Melbourne, Victoria (AU); Olivia Newton-John Cancer Research Institute, Heidelberg, Victoria (AU); CSL INNOVATION PTY LTD, Parkville, Victoria (AU)

(21) Appl. No.: 17/622,563

(22) PCT Filed: Jun. 26, 2020
(86) PCT No.: PCT/AU2020/050662

§ 371 Date: Dec. 23, 2021

(30) Foreign Application Priority Data
Jun. 28, 2019 (AU) 2019902308
Dec. 17, 2019 (AU) 2019904771
Dec. 17, 2019 (AU) 2019904773

(51) Int. Cl. A61K 35/17; C07K 16/28; C07K 14/725; A61P 31/00; A61P 35/00; A61P 37/00

(52) U.S. Cl. CPC A61K 35/17; C07K 16/2803; C07K 14/7051; A61P 31/00; A61P 35/00; A61P 37/00

(57) ABSTRACT

The present disclosure relates to methods for inhibiting activation of γδ T cells that express a Vγ9+ TCR in a subject by administering a BTN2A1 antagonist to a subject as well as methods for inducing or enhancing γδ T cells that express a Vγ9+ TCR in a subject by administering a BTN2A1 antagonist to a subject. The disclosure additionally relates to BTN2A1 antagonists and BTN2A1 agonists.

### RELATED APPLICATION DATA

The present application claims priority from Australian Patent Application No. 2019902308 entitled “Methods of inhibiting or activating gamma delta T cells” filed on 28 Jun. 2019, Australian Patent Application No. 2019904771 entitled “Methods of inhibiting or activating gamma delta T cells” filed 17 Dec. 2019 and Australian Patent Application No. 2019904773 entitled “Methods of inhibiting or activating gamma delta T cells” filed 17 Dec. 2019. The entire contents of each application is hereby incorporated by reference.

### SEQUENCE LISTING

The present application is filed with a Sequence Listing in electronic form. The entire contents of the Sequence Listing are hereby incorporated by reference.

### FIELD

The present disclosure relates to reagents and methods for inhibiting or activating γδ T cells.

### INTRODUCTION

Alpha-beta (αβ) T cells recognize antigens (Ag) via T cell receptors (TCRs) encoded by TCR-α and TCR-β gene loci, that bind to Ag displayed by Ag-presenting molecules. This fundamental principle applies to αβ T cells that recognize peptide Ags presented by MHC molecules, NKT cells that recognize lipid Ags presented by CD1d, and mucosal-associated invariant T (MAIT) cells that recognize vitamin B metabolites presented by MRI (J. Rossjohn et al. (2015)). Gamma-delta (γδ) T cells are a unique lineage that expresses TCRs derived from separate variable (V), diversity (D), joining (J) and constant (C) TCR-γ and TCR-δ gene loci. Most circulating human γδ T cells express a Vγ9⁺ TCR, and most of these react to a distinct class of Ag, termed phosphoantigens (pAg) (P. Constant et al. (1994); Y. Tanaka et al., (1995)).

pAgs are intermediates in the biosynthesis of isoprenoids, present in virtually all cellular organisms. While vertebrates produce isoprenoids via the mevalonate pathway, microbes utilize the non-mevalonate pathway, which yields chemically distinct pAg intermediates (L. Zhao et al. (2013)). Vγ9⁺ T cells sense pAgs produced via either pathway, including isopentenyl pyrophosphate (IPP) from the mevalonate pathway and 4-hydroxy-3-methyl-but-2-enyl pyrophosphate (HMBPP) from the non-mevalonate pathway, but with ˜1000-fold higher sensitivity for microbial HMBPP than vertebrate IPP pAgs (A. Sandstrom et al. (2014)). Thus, they can respond to HMBPP derived from microbial infection, but also accumulated IPP in abnormal cells such as cancer cells. During bacterial and parasitic infections, pAg drives Vγ9⁺ T cells to produce cytokines and expand to represent ˜10%-50% of peripheral blood mononuclear cells (PBMCs) (Y. L. Wu et al. (2014); J. Zheng et al. (2013)). The important role that Vγ9⁺ T cells play in anti-bacterial immunity was demonstrated by human PBMC transfer into immune-deficient mice, which led to Vγ9 T cell-dependent protection against bacterial infection (L. Wang et al. (2001)). They can also kill diverse tumor cell lines in vitro in a pAg-dependent manner, and numerous clinical trials have examined their anti-cancer potential, with some encouraging results (D. I. Godfrey et al. (2018)). Accordingly, Vγ9+ γδ T cells represent a critical and non-redundant arm of the human immune system.

Despite the importance of pAg sensing by γδ T cells in protective immunity, the molecular mechanisms that govern pAg recognition are unclear.

It will be clear to the skilled person from the foregoing that there is a need to better understand the mechanisms that govern pAg recognition to provide novel immunotherapies and agents that can induce or inhibit γδ T cell responses in, for example, cancer patients, or patients with chronic infections.

### SUMMARY

In arriving at the present invention, the inventors identified the surface protein butyrophilin, subfamily 2, member A1 (BTN2A1) as a novel ligand for the pAg-reactive γδTCR. The inventors demonstrated that BTN2A1 expression is obligatory for effective pAg responses by γδ T cells. The inventors also showed that BTN2A1 closely associates with BTN3A1 on the surface of antigen presenting cells (APCs) and this complex is necessary and sufficient to confer mouse and hamster APC with pAg-presenting capacity.

These findings by the inventors provide the basis for reagents that bind to BTN2A1 and enhance γδ T activation, and their use in the treatment of, for example, cancer or infection.

These findings by the inventors also provide the basis for reagents that bind to BTN2A1 and disrupt γδ T activation, and their use in the treatment of, for example, autoimmune disease, transplantation rejection, or graft versus host disease.

Accordingly, the present disclosure provides a method for inhibiting activation of γδ T cells that express a Vγ9+ TCR in a subject, the method comprising administering a BTN2A1 antagonist to the subject, wherein the BTN2A1 antagonist:
i) inhibits formation of a BTN2A1/BTN3 complex, for example, a BTN2A1/BTN3A1 complex on the surface of a cell;
ii) inhibits binding of BTN2A1 to Vγ9;
iii) inhibits binding of a BTN2A1/BTN3, for example, a BTN2A1/BTN3A1 complex to the Vγ9+ TCR; and/or
iv) decreases the activity and/or survival of cells that express BTN2A1.

In an embodiment, the method inhibits activation of one or more Vγ9+ T cell subsets. For example, the method inhibits activation of one or more of Vγ9Vδ2+, Vγ9Vδ1+, Vγ9Vδ3+, Vγ9Vδ4+, or Vγ9Vδ5+ γδ T cells. In another example, the method inhibits activation of Vγ9Vδ2− T cells. For example, the method inhibits activation of one or more of Vγ9Vδ2+, Vγ9Vδ1+, Vγ9Vδ3+, Vγ9Vδ4+, or Vγ9Vδ5+ γδ or Vγ9Vδ2− T cells. For example, the method inhibits CD25 upregulation on the surface of one or more Vγ9+ T cell subsets and/or production of IFN-γ therefrom. In an embodiment, the method inhibits activation of Vγ9Vδ2+ γδ T cells. In another embodiment, the method inhibits activation of Vγ9Vδ2− γδ T cells. In a further embodiment, the method inhibits activation of Vγ9Vδ2+ γδ T cells and/or Vγ9Vδ2− γδ T cells.

In an embodiment, the BTN2A1/BTN3 is a BTN2A1/BTN3A1 complex. The complex may be a heteromeric complex or a multimeric complex.

In an embodiment, BTN2A1 and BTN3 are expressed on the same cell.

In a further embodiment, the BTN2A1/BTN3A1 complex comprises one or more additional molecules such as BTN3A2 and/or BTN3A3. The one or more additional molecules may enhance activation of the γδ T cells.

In an embodiment, the method inhibits one or more of cytolytic function, cytokine production of one or more cytokines, or proliferation of the γδ T cells.

In an embodiment, the BTN2A1 antagonist inhibits phosphoantigen mediated activation of the γδ T cells.

In an embodiment or a further embodiment, the BTN2A1 antagonist inhibits association of BTN2A1 and BTN3A1, for example, the BTN2A1 antagonist inhibits direct association of BTN2A1 and BTN3A1.

In an embodiment or a further embodiment, the BTN2A1 antagonist inhibits binding of BTN2A1 to the germline-encoded region of Vγ9 and/or distal to the TCR δ-chain. In an embodiment, the BTN2A1 antagonist prevents binding of BTN2A1 to a framework region and/or a region including at least one of Arg20, Glu70 and His85 of Vγ9. The BTN2A1 antagonist may prevent binding to a region on the outer faces of the B, D, and E strands of the ABED antiparallel β-sheet of Vγ9. In an embodiment, the BTN2A1 antagonist binds to a region that is closer to the Cγ domain than the CDR loops.

In an embodiment, the BTN2A1 antagonist inhibits binding of a BTN2A1/BTN3 complex to the germline-encoded regions of Vδ2 such as the CDR2 loop of the TCR δ chain and/or the CDR3 loop of the TCR γ chain. For example, the BTN2A1 antagonist prevents binding of BTN2A1 to a region in proximity of Arg51 of Vδ2 and Lys108 of Vγ9−JγP-encoded CDR3 loop.

In an embodiment, the BTN2A1 antagonist modifies one or more of the extracellular domains (IgV and/or IgC) of the BTN2A1 molecule to switch the BTN2A1 molecule from stimulatory BTN2A1 to that of non-stimulatory.

In an embodiment, or a further embodiment, the BTN2A1 antagonist modifies one or more of the extracellular domains (IgV and/or IgC) of the BTN2A1 molecule and inhibits phosphoantigen activation. For example, the BTN2A1 antagonist inhibits binding of the phosphoantigen to a cytoplasmic domain of BTN2A1 and/or a BTN3 molecule.

In an embodiment, the BTN2A1 antagonist is bi-specific for BTN2A1 and a BTN3 molecule, for example, BTN3A1. In another embodiment, the BTN2A1 antagonist cross-reacts with a BTN3 molecule, for example, BTN3A1. In another embodiment, the BTN2A1 antagonist is a soluble Vγ9+ TCR.

The present disclosure also provides a method of suppressing or inhibiting Vγ9+ γδ T cell responses in a subject, wherein the method comprises administering a BTN2A1 antagonist to the subject, wherein the BTN2A1 antagonist:
i) inhibits formation of a BTN2A1/BTN3 complex, for example, a BTN2A1/BTN3A1 complex on the surface of a cell;
ii) inhibits binding of BTN2A1 to Vγ9+ TCR;
iii) inhibits binding of a BTN2A1/BTN3 complex, for example, a BTN2A1/BTN3A1 complex to the Vγ9+ TCR; and/or
iv) decreases the activity and/or survival of cells that express BTN2A1.

In an embodiment, the method suppresses or inhibits one or more one or more of Vγ9Vδ2+, Vγ9Vδ1+, Vγ9Vδ3+, Vγ9Vδ4+, or Vγ9Vδ5+ γδ T cell responses. In an embodiment, the method suppresses or inhibits one or more one or more of Vγ9Vδ2+, Vγ9Vδ2−, Vγ9Vδ1+, Vγ9Vδ3+, Vγ9Vδ4+, or Vγ9Vδ5+ γδ T cell responses. In an embodiment, the method suppresses or inhibits Vγ9Vδ2+ γδ T cell responses. In another embodiment, the method suppresses or inhibits Vγ9Vδ2−γδ T cell responses. In a further embodiment, the method suppresses or inhibits Vγ9Vδ2+ γδ T cell responses and/or Vγ9Vδ2− γδ T cell responses.

In an embodiment, the BTN2A1/BTN3 is a BTN2A1/BTN3A1 complex. The complex may be a heteromeric complex or a multimeric complex.

In a further embodiment, the BTN2A1/BTN3A1 complex comprises one or more additional molecules such as BTN3A2 and/or BTN3A3. The one or more additional molecules may enhance activation of the γδ T cells.

In an embodiment, the method suppresses or inhibits one or more of cytolytic function, cytokine production of one or more cytokines, or proliferation of the γδ T cells.

In an embodiment, the BTN2A1 antagonist inhibits phosphoantigen mediated activation of the γδ T cells.

In an embodiment or a further embodiment, the BTN2A1 antagonist inhibits association of BTN2A1 and BTN3A1, for example, the BTN2A1 antagonist inhibits direct association of BTN2A1 and BTN3A1.

In an embodiment or a further embodiment, the BTN2A1 antagonist inhibits binding of BTN2A1 to the germline-encoded region of Vγ9 and/or distal to the TCR δ-chain. In an embodiment, the BTN2A1 antagonist prevents binding of BTN2A1 to a framework region and/or a region including at least one of Arg20, Glu70 and His85 of Vγ9. The BTN2A1 antagonist may prevent binding to a region on the outer faces of the B, D, and E strands of the ABED antiparallel β-sheet of Vγ9. In an embodiment, the BTN2A1 antagonist binds to a region that is closer to the Cγ domain than the CDR loops.

In an embodiment, the BTN2A1 antagonist inhibits binding of a BTN2A1/BTN3 complex to the germline-encoded regions of Vδ2 such as the CDR2 loop and/or the CDR3 loop of the TCR γ chain. For example, the BTN2A1 antagonist prevents binding of BTN2A1 to a region in proximity of at least one of Arg51 and Lys108 of Vγ9−JγP-encoded CDR3 loop.

In an embodiment, the BTN2A1 antagonist modifies one or more of the extracellular domains (IgV and/or IgC) of the BTN2A1 molecule to switch the BTN2A1 molecule from stimulatory BTN2A1 to that of non-stimulatory.

In an embodiment, or a further embodiment, the BTN2A1 antagonist modifies one or more of the extracellular domains (IgV and/or IgC) of the BTN2A1 molecule and inhibits phosphoantigen activation. For example, the BTN2A1 antagonist inhibits binding of the phosphoantigen to a cytoplasmic domain of BTN2A1 and/or a BTN3 molecule.

In an embodiment, the BTN2A1 antagonist is bi-specific for BTN2A1 and a BTN3 molecule, for example, BTN3A1. In another embodiment, the BTN2A1 antagonist cross-reacts with a BTN3 molecule, for example, BTN3A1. In another embodiment, the BTN2A1 antagonist is a soluble Vγ9+ TCRs.

The present disclosure also provides a method for inhibiting activation of γδ T cells that express a Vγ9+ TCR in vitro or ex vivo, the method comprising culturing the γδ T cells and cells expressing BTN2A1 in the presence of a BTN2A1 antagonist, wherein the BTN2A1 antagonist:
i) inhibits formation of a BTN2A1/BTN3A1 heteromeric complex on the surface of the cells;
ii) inhibits binding of BTN2A1 to Vγ9;
iii) inhibits binding of a BTN2A1/BTN3A1 heteromeric complex to the Vγ9+ TCR; and/or
iv) decreases the activity and/or survival of cells that express BTN2A1.

In an embodiment, the method further comprises the step of administering the γδ T cells to a subject in need thereof. For example, the γδ T cells comprise an engineered receptor, e.g., a genetically engineered or modified T cell receptor. For example, the γδ T cells do not comprise an engineered receptor, e.g., a genetically engineered or modified T cell receptor. In a further embodiment, the γδ T cells are engineered γδ T cells. This method may be useful in the context of treating a patient with a tissue graft or allogeneic blood cell graft.

The present disclosure also provides a method of preventing, treating, delaying the progression of, preventing a relapse of, or alleviating a symptom of an autoimmune disease, transplantation rejection, graft versus host disease, or graft versus tumour effect, the method comprising administering a BTN2A1 antagonist to a subject in need thereof in an amount sufficient to prevent, treat, delay the progression of, prevent a relapse of, or alleviate the symptom of the autoimmune disease, transplant rejection or graft versus host disease, or graft versus tumour effect in the subject.

The present disclosure also provides a method of preventing, treating, delaying the progression of, preventing a relapse of, or alleviating a symptom of a cancer or an infection, the method comprising administering a BTN2A1 antagonist to a subject in need thereof in an amount sufficient to prevent, treat, delay the progression of, prevent a relapse of, or alleviate the symptom of the cancer or infection in the subject.

The present disclosure also provides a method for activating γδ T cells that express a Vγ9+ TCR in a subject, the method comprising administering a BTN2A1 agonist to the subject, wherein the BTN2A1 agonist:
i) promotes formation of a BTN2A1/BTN3, for example, a BTN2A1/BTN3A1 complex on the surface of a cell;
ii) induces ligation of Vγ9+ TCR on γδ T cells; and/or
iii) increases the activity and/or survival of cells that express BTN2A1.

In an embodiment, the method activates one or more Vγ9+ T cell subsets. For example, one or more of Vγ9Vδ2+, Vγ9Vδ1+, Vγ9Vδ3+, Vγ9Vδ4+, or Vγ9Vδ5+ γδ T cells. For example, one or more of Vγ9Vδ2+, Vγ9Vδ2−, Vγ9Vδ1+, Vγ9Vδ3+, Vγ9Vδ4+, or Vγ9Vδ5+ γδ T cells. In an embodiment, the method activates Vγ9Vδ2+ γδ T cells. In another embodiment, the method activates Vγ9Vδ2− γδ T cells. In a further embodiment, the method activates Vγ9Vδ2+ γδ T cells and Vγ9Vδ2− γδ T cells.

In an embodiment, the BTN2A1/BTN3 is a BTN2A1/BTN3A1 complex. The complex may be a heteromeric complex or a multimeric complex.

In a further embodiment, the BTN2A1/BTN3A1 complex comprises one or more additional molecules such as BTN3A2 and/or BTN3A3. The one or more additional molecules may enhance activation of the γδ T cells.

In an embodiment, the method activates one or more of cytolytic function, cytokine production of one or more cytokines, or proliferation of the γδ T cells.

In an embodiment, or a further embodiment, the activated γδ T cells express one or more of CD25, CD40-Ligand (CD40-L), CD69 and CD107a.

In an embodiment, the BTN2A1 agonist activates the γδ T cells independent of phosphoantigen binding.

In an embodiment or a further embodiment, the BTN2A1 agonist promotes association of BTN2A1 and BTN3A1, for example, the BTN2A1 agonist promotes direct association of BTN2A1 and BTN3A1. For example, the BTN2A1 agonist cross-links BTN2A1 and BTN3A1.

In an embodiment, the BTN2A1 agonist is bi-specific for BTN2A1 and a BTN3 molecule, for example, BTN3A1. In another embodiment, the BTN2A1 agonist cross-reacts with a BTN3 molecule, for example, BTN3A1.

In an embodiment, the BTN2A1 agonist modifies one or more of the extracellular domains (IgV and/or IgC) of the BTN2A1 molecule to switch the BTN2A1 from non-stimulatory BTN2A1 to that of stimulatory.

The present disclosure also provides a method of inducing or enhancing Vγ9+ γδ T cell responses in a subject, wherein the method comprises administering a BTN2A1 agonist to the subject, wherein the BTN2A1 agonist:
i) promotes formation of a BTN2A1/BTN3, for example, a BTN2A1/BTN3A1 complex on the surface of a cell;
ii) induces ligation of Vγ9+ TCR on γδ T cells; and/or
iii) increases the activity and/or survival of cells that express BTN2A1.

In an embodiment, the method induces one or more Vγ9+ T cell subsets. For example, one or more of Vγ9Vδ2+, Vγ9Vδ1+, Vγ9Vδ3+, Vγ9Vδ4+, or Vγ9Vδ5+ γδ T cells. For example, one or more of Vγ9Vδ2+, Vγ9Vδ2− γδ, Vγ9Vδ1+, Vγ9Vδ3+, Vγ9Vδ4+, or Vγ9Vδ5+ γδ T cells. In an embodiment, the method induces Vγ9Vδ2+ γδ T cell responses. In another embodiment, the method induces Vγ9Vδ2− γδ T cell responses. In a further embodiment, the method induces Vγ9Vδ2+ γδ T cell and Vγ9Vδ2− γδ T cell responses.

In an embodiment, the BTN2A1/BTN3 is a BTN2A1/BTN3A1 complex. The complex may be a heteromeric complex or a multimeric complex.

In a further embodiment, the BTN2A1/BTN3A1 complex comprises one or more additional molecules such as BTN3A2 and/or BTN3A3. The one or more additional molecules may enhance activation of the γδ T cells.

In an embodiment, the method activates one or more of cytolytic function, cytokine production of one or more cytokines, or proliferation of the γδ T cells.

In an embodiment, or a further embodiment, the activated γδ T cells express one or more activation associated markers such as of CD25, CD69, CD40-Ligand (CD40-L) and CD107a.

In an embodiment, the BTN2A1 agonist activates the γδ T cells independent of phosphoantigen binding.

In an embodiment or a further embodiment, the BTN2A1 agonist promotes association of BTN2A1 and BTN3A1, for example, the BTN2A1 agonist promotes direct association of BTN2A1 and BTN3A1. For example, the BTN2A1 agonist cross-links BTN2A1 and BTN3A1.

In an embodiment, the BTN2A1 agonist is bi-specific for BTN2A1 and a BTN3 molecule, for example, BTN3A1. In another embodiment, the BTN2A1 agonist cross-reacts with a BTN3 molecule, for example, BTN3A1.

In an embodiment, the BTN2A1 agonist modifies one or more of the extracellular domains (IgV and/or IgC) of the BTN2A1 molecule to switch the BTN2A1 from non-stimulatory BTN2A1 to that of stimulatory.

The present disclosure also provides a method for activating γδ T cells that express a Vγ9+ TCR in vitro or ex vivo, the method comprising culturing the γδ T cells and cells expressing BTN2A1 in the presence of a BTN2A1 agonist, wherein the BTN2A1 agonist:
i) promotes formation of a BTN2A1/BTN3A1 heteromeric complex on the surface of antigen presenting cells;
ii) induces ligation of Vγ9+ TCR on γδ T cells; and/or
iii) increases the activity and/or survival of cells that express BTN2A1.

In an embodiment, the method further comprises the step of administering the activated γδ T cells to a subject in need thereof. In a further embodiment, the method further comprises the step of administering the engineered γδ T cells to a subject in need thereof.

The present disclosure also provides a method of preventing, treating, delaying the progression of, preventing a relapse of, or alleviating a symptom of an autoimmune disease, transplantation rejection, graft versus host disease, or graft versus tumour effect, the method comprising administering a BTN2A1 agonist to a subject in need thereof in an amount sufficient to prevent, treat, delay the progression of, prevent a relapse of, or alleviate the symptom of the autoimmune disease, transplant rejection, graft versus host disease, or graft versus tumour effect in the subject.

The present disclosure also provides a method of preventing, treating, delaying the progression of, preventing a relapse of, or alleviating a symptom of a cancer or an infection, the method comprising administering a BTN2A1 agonist to a subject in need thereof in an amount sufficient to prevent, treat, delay the progression of, prevent a relapse of, or alleviate the symptom of the cancer or infection in the subject.

The present disclosure also provides a BTN2A1 antagonist, wherein the BTN2A1 antagonist specifically binds to BTN2A1 and inhibits:
i) formation of a BTN2A1/BTN3 complex, for example, a BTN2A1/BTN3A1 complex on the surface of a cell;
ii) binding of BTN2A1 to Vγ9;
iii) binding of a BTN2A1/BTN3A1 complex to the Vγ9+ TCR; and/or
iv) decreases the activity and/or survival of cells that express BTN2A1.

The present disclosure also provides a BTN2A1 agonist specifically binds to BTN2A1 and:
i) promotes formation of a BTN2A1/BTN3 complex, for example, a BTN2A1/BTN3A1 complex on the surface of a cell;
ii) induces ligation of Vγ9+ TCR on γδ T cells; and/or
iii) increases the activity and/or survival of cells that express BTN2A1.

In an embodiment, the BTN2A1 antagonist or agonist is a protein comprising an antigen binding domain.

In an embodiment, the protein is:
(i) a single chain Fv fragment (scFv);
(ii) a dimeric scFv;
(iii) a Fv fragment;
(iv) a single domain antibody (sdAb) (for example, a nanobody);
(v) a diabody, triabody, tetrabody or higher order multimer;
(vi) Fab fragment;
(vii) a Fab′ fragment;
(viii) a F(ab′) fragment;
(ix) a F(ab′)₂ fragment;
(x) any one of (i)-(ix) linked to a Fc region of an antibody;
(xi) any one of (i)-(ix) fused to an antibody or antigen binding fragment thereof that binds to an immune effector cell; or
(xii) an antibody.

In an embodiment, the protein is:
(i) a single chain Fv fragment (scFv);
(ii) a dimeric scFv;
(iii) a Fv fragment;
(iv) a single domain antibody (sdAb);
(v) a nanobody;
(vi) a diabody, triabody, tetrabody or higher order multimer;
(vii) Fab fragment;
(viii) a Fab′ fragment;
(ix) a F(ab′) fragment;
(x) a F(ab′)₂ fragment;
(xi) any one of (i)-(x) linked to a Fc region of an antibody;
(xii) any one of (i)-(x) fused to an antibody or antigen binding fragment thereof that binds to an immune effector cell; or
(xiii) an antibody.

In one example, the protein of the present disclosure is an affinity matured, chimeric, CDR grafted, or humanized antibody, or antigen binding fragment thereof.

In one example, the BTN2A1 antagonist is an antibody comprising a heavy chain variable region (V_{H}) comprising a sequence set forth in SEQ ID NO: 100 and a light chain variable region (V_{L}) comprising a sequence set forth in SEQ ID NO: 101.

In another example, the BTN2A1 antagonist is an antibody comprising a heavy chain variable region (V_{H}) comprising a sequence set forth in SEQ ID NO: 108 and a light chain variable region (V_{L}) comprising a sequence set forth in SEQ ID NO: 109.

In another example, the BTN2A1 antagonist is an antibody comprising a heavy chain variable region (V_{H}) comprising a sequence set forth in SEQ ID NO: 116 and a light chain variable region (V_{L}) comprising a sequence set forth in SEQ ID NO: 117.

In another example, the BTN2A1 antagonist is an antibody comprising a heavy chain variable region (V_{H}) comprising a sequence set forth in SEQ ID NO: 124 and a light chain variable region (V_{L}) comprising a sequence set forth in SEQ ID NO: 125.

In another example, the BTN2A1 antagonist is an antibody comprising a heavy chain variable region (V_{H}) comprising a sequence set forth in SEQ ID NO: 132 and a light chain variable region (V_{L}) comprising a sequence set forth in SEQ ID NO: 133.

In one example, the BTN2A1 antagonist is an antibody comprising a V_{H} Comprising the complementarity determining regions (CDRs) of a V_{H} comprising an amino acid sequence set forth in SEQ ID NO: 100 and a V_{L} comprising the CDRs of a V_{L} comprising an amino acid sequence set forth in SEQ ID NO: 101.

For example, the antagonist is an antibody comprising:

(i) a V_{H} comprising:
(a) a CDR1 comprising a sequence set forth in amino acids 26-33 of SEQ ID NO: 100;
(b) a CDR2 comprising a sequence set forth in amino acids 51-58 of SEQ ID NO: 100; and
(c) a CDR3 comprising a sequence set forth in amino acids 97-105 of SEQ ID NO: 100; and/or
   (ii) a V_{L} comprising:
(a) a CDR1 comprising a sequence set forth in amino acids 27-32 of SEQ ID NO: 101;
(b) a CDR2 comprising a sequence set forth in amino acids 50-52 of SEQ ID NO: 101; and
(c) a CDR3 comprising a sequence set forth in amino acids 89-97 of SEQ ID NO: 101.

In one example, the antagonist is an antibody comprising:

(i) a V_{H} comprising:
(a) a CDR1 comprising a sequence set forth in SEQ ID NO: 102;
(b) a CDR2 comprising a sequence set forth in SEQ ID NO: 103; and
(c) a CDR3 comprising a sequence set forth in SEQ ID NO: 104; and/or
   (ii) a V_{L} comprising:
(a) a CDR1 comprising a sequence as set forth in SEQ ID NO: 105;
(b) a CDR2 comprising a sequence as set forth in SEQ ID NO: 106; and
(c) a CDR3 comprising a sequence as set forth in SEQ ID NO: 107.

In another example, the antagonist is an antibody comprising:

(i) a V_{H} comprising:
(a) a CDR1 comprising a sequence set forth in amino acids 26-33 of SEQ ID NO: 108;
(b) a CDR2 comprising a sequence set forth in amino acids 51-58 of SEQ ID NO: 108; and
(c) a CDR3 comprising a sequence set forth in amino acids 97-105 of SEQ ID NO: 108; and/or
   (ii) a V_{L} comprising:
(a) a CDR1 comprising a sequence set forth in amino acids 27-33 of SEQ ID NO: 109;
(b) a CDR2 comprising a sequence set forth in amino acids 51-53 of SEQ ID NO: 109; and
(c) a CDR3 comprising a sequence set forth in amino acids 90-98 of SEQ ID NO: 109.

In one example, the antagonist is an antibody comprising:

(i) a V_{H} comprising:
(a) a CDR1 comprising a sequence set forth in SEQ ID NO: 110;
(b) a CDR2 comprising a sequence set forth in SEQ ID NO: 111; and
(c) a CDR3 comprising a sequence set forth in SEQ ID NO: 112; and/or
   (ii) a V_{L} comprising:
(a) a CDR1 comprising a sequence as set forth in SEQ ID NO: 113;
(b) a CDR2 comprising a sequence as set forth in SEQ ID NO: 114; and
(c) a CDR3 comprising a sequence as set forth in SEQ ID NO: 115.

In another example, the antagonist is an antibody comprising:

(i) a V_{H} comprising:
(a) a CDR1 comprising a sequence set forth in amino acids 26-33 of SEQ ID NO: 116;
(b) a CDR2 comprising a sequence set forth in amino acids 51-58 of SEQ ID NO: 116; and
(c) a CDR3 comprising a sequence set forth in amino acids 97-104 of SEQ ID NO: 116; and/or
   (ii) a V_{L} comprising:
(a) a CDR1 comprising a sequence set forth in amino acids 27-32 of SEQ ID NO: 117;
(b) a CDR2 comprising a sequence set forth in amino acids 24-26 of SEQ ID NO: 117; and
(c) a CDR3 comprising a sequence set forth in amino acids 89-97 of SEQ ID NO: 117.

In one example, the antagonist is an antibody comprising:

(i) a V_{H} comprising:
(a) a CDR1 comprising a sequence set forth in SEQ ID NO: 118;
(b) a CDR2 comprising a sequence set forth in SEQ ID NO: 119; and
(c) a CDR3 comprising a sequence set forth in SEQ ID NO: 120; and/or
   (ii) a V_{L} comprising:
(a) a CDR1 comprising a sequence as set forth in SEQ ID NO: 121;
(b) a CDR2 comprising a sequence as set forth in SEQ ID NO: 122; and
(c) a CDR3 comprising a sequence as set forth in SEQ ID NO: 123.

In another example, the antagonist is an antibody comprising:

(i) a V_{H} comprising:
(a) a CDR1 comprising a sequence set forth in amino acids 26-33 of SEQ ID NO: 124;
(b) a CDR2 comprising a sequence set forth in amino acids 51-58 of SEQ ID NO: 124; and
(c) a CDR3 comprising a sequence set forth in amino acids 97-105 of SEQ ID NO: 124; and/or
   (ii) a V_{L} comprising:
(a) a CDR1 comprising a sequence set forth in amino acids 26-33 of SEQ ID NO: 125;
(b) a CDR2 comprising a sequence set forth in amino acids 51-53 of SEQ ID NO: 125; and
(c) a CDR3 comprising a sequence set forth in amino acids 90-101 of SEQ ID NO: 125.

In one example, the antagonist is an antibody comprising:

(i) a V_{H} comprising:
(a) a CDR1 comprising a sequence set forth in SEQ ID NO: 126;
(b) a CDR2 comprising a sequence set forth in SEQ ID NO: 127; and
(c) a CDR3 comprising a sequence set forth in SEQ ID NO: 128; and/or
   (ii) a V_{L} comprising:
(a) a CDR1 comprising a sequence as set forth in SEQ ID NO: 129;
(b) a CDR2 comprising a sequence as set forth in SEQ ID NO: 130; and
(c) a CDR3 comprising a sequence as set forth in SEQ ID NO: 131.

In another example, the antagonist is an antibody comprising:

(i) a V_{H} comprising:
(a) a CDR1 comprising a sequence set forth in amino acids 26-33 of SEQ ID NO: 132;
(b) a CDR2 comprising a sequence set forth in amino acids 51-58 of SEQ ID NO: 132; and
(c) a CDR3 comprising a sequence set forth in amino acids 97-106 of SEQ ID NO: 132; and/or
   (ii) a V_{L} comprising:
(a) a CDR1 comprising a sequence set forth in amino acids 26-33 of SEQ ID NO: 133;
(b) a CDR2 comprising a sequence set forth in amino acids 51-53 of SEQ ID NO: 133; and
(c) a CDR3 comprising a sequence set forth in amino acids 92-100 of SEQ ID NO: 133.

In one example, the antagonist is an antibody comprising:

(i) a V_{H} comprising:
(a) a CDR1 comprising a sequence set forth in SEQ ID NO: 134;
(b) a CDR2 comprising a sequence set forth in SEQ ID NO: 135; and
(c) a CDR3 comprising a sequence set forth in SEQ ID NO: 136; and/or
   (ii) a V_{L} comprising:
(a) a CDR1 comprising a sequence as set forth in SEQ ID NO: 137;
(b) a CDR2 comprising a sequence as set forth in SEQ ID NO: 138; and
(c) a CDR3 comprising a sequence as set forth in SEQ ID NO: 139.

In one example, the protein of the present disclosure is an affinity matured, chimeric, CDR grafted, or humanized antibody, or antigen binding fragment thereof.

In one example, the protein, antibody or antigen binding fragment thereof is any form of the protein, antibody or functional fragment thereof encoded by a nucleic acid encoding any of the foregoing proteins, antibodies or functional fragments

In one example, the antagonist is a protein, for example, an antibody comprising a variable region that competitively inhibits the binding of an antibody disclosed herein.

In another embodiment, the BTN2A1 antagonist is a soluble Vγ9+ TCR. The soluble Vγ9+ TCR can comprise any TCR allele.

In an embodiment, the soluble Vγ9+ TCR is a monomer.

In an embodiment, the soluble Vγ9+ TCR is a multimer.

In an embodiment, the soluble Vγ9+ TCR comprises a γ chain comprising a sequence set forth in any one of SEQ ID NO:85-89 and/or a δ chain comprising a sequence set forth in any one of SEQ ID NO:70-74. In an embodiment, the γ and δ chains are cleaved, for example, at the thrombin protease cleavage site (e.g., LVPRGS).

In an embodiment, the soluble Vγ9+ TCR comprises a γ chain comprising a variable region comprising a sequence set forth in any one of SEQ ID NO:90-94 and/or a δ chain comprising a variable region comprising a sequence set forth in any one of SEQ ID NO:75-79.

In an embodiment, the soluble Vγ9+ TCR comprises the complementarity determining region 3 (CDR3) of the γ chain variable region comprising a sequence set forth in any one of SEQ ID NO:95-99 and/or a complementarity determining region 3 (CDR3) of the δ chain variable region comprising a sequence set forth in any one of SEQ ID NO:80-84.

In an embodiment, the soluble Vγ9+ TCR comprises a γ chain variable region comprising a CDR3 set forth in any one of SEQ ID NO:95-99 and/or a δ chain variable region comprising a CDR set forth in any one of SEQ ID NO:80-84.

In an embodiment, the soluble Vγ9+ TCR comprises a γ chain variable region comprising a CDR3 set forth in SEQ ID NO:95 and a δ chain variable region comprising a CDR3 set forth in SEQ ID NO:80.

In an embodiment, the soluble Vγ9+ TCR comprises a γ chain variable region comprising a CDR3 set forth in SEQ ID NO:96 and a δ chain variable region comprising a CDR3 set forth in SEQ ID NO:81.

In an embodiment, the soluble Vγ9+ TCR comprises a γ chain variable region comprising a CDR3 set forth in SEQ ID NO:97 and a δ chain variable region comprising a CDR3 set forth in SEQ ID NO:82.

In an embodiment, the soluble Vγ9+ TCR comprises a γ chain variable region comprising a CDR3 set forth in SEQ ID NO:98 and a δ chain variable region comprising a CDR3 set forth in SEQ ID NO:83.

In an embodiment, the soluble Vγ9+ TCR comprises a γ chain variable region comprising a CDR3 set forth in SEQ ID NO:99 and a δ chain variable region comprising a CDR3 set forth in SEQ ID NO:84.

The present disclosure additionally provides a BTN2A1 agonist, wherein the BTN2A1 agonist specifically binds to BTN2A1 and leads to the activation of γδ T cells.

The present disclosure additionally provides a BTN2A1 agonist, wherein the BTN2A1 agonist specifically binds to BTN2A1 and induces expression of a cell surface markers associated with γδ T cell activation.

The present disclosure additionally provides a BTN2A1 agonist, wherein the BTN2A1 agonist specifically binds to BTN2A1 and induces secretion of a cytokine, or cytokines, by γδ T cells.

The present disclosure additionally provides a BTN2A1 agonist, wherein the BTN2A1 agonist specifically binds to BTN2A1 and induces the γδ T cells to kill a cancer cell and/or inhibit growth of the cancer cell and/or kill a cell infected with, e.g., a virus, bacteria or parasite and/or inhibit growth of a cell infected with e.g., a virus, bacteria or parasite.

The present disclosure additionally provides a BTN2A1 agonist, wherein the BTN2A1 agonist specifically binds to BTN2A1 and:

(i) activates γδ T cells and/or increases the number of activated γδ T cells in a population of cells; and/or
(i) increases the percentage of γδ T cells expressing a marker of T cell activation; and/or
(ii) increases secretion of a cytokine (e.g., interferon-γ) by γδ T cells; and/or
(iii) induces γδ T cells to kill cancer cells and/or inhibit growth of the cancer cells and/or kill infected cells and/or inhibit growth of infected cells; and/or
(iv) increases the amount of a marker of T cell activation expressed on the cell surface of γδ T cells.

The present disclosure additionally provides a BTN2A1 agonist, wherein the BTN2A1 agonist specifically binds to BTN2A1 and:

(i) increases the percentage of γδ T cells expressing CD25 on the cell surface; and/or
(ii) increases secretion of interferon γ by γδ T cells; and/or
(iii) induces γδ T cells to kill cancer cells and/or inhibit growth of the cancer cells; and/or
(iv) increases the amount of CD25 expressed on the cell surface of γδ T cells.

In one example, the BTN2A1 agonist increases the number of γδ T cells expressing CD25 on the cell surface as measured in an assay comprising contacting a population of γδ T cells in vitro with the BTN2A1 agonist for a period of at least 6 hours or 8 hours or 10 hours or 12 hours and measuring the percentage of γδ T cells in the population expressing CD25 with flow cytometry. Such assays are also useful for assessing the level of CD25 and or other molecules expressed on γδ T cells.

In one example, the increase in the percentage of γδ T cells expressing CD25 on the cell surface is relative to:

(i) the percentage of γδ T cells expressing CD25 on the cell surface in a population of γδ T cells that have not been contacted with the BTN2A1 agonist; and/or
(ii) the percentage of γδ T cells expressing CD25 on the cell surface in a population of γδ T cells that have been contacted with an antibody that binds specifically to BTN2A1 that is not a BTN2A1 agonist or a BTN2A1 antagonist.

In one example, the agonist increases the percentage of γδ T cells expressing one or more additional markers (additional to CD25) of activation of γδ T cells and/or increases the amount of one or more additional markers of activation CD25 (additional to CD25) expressed on the cell surface of γδ T cells.

In one example, the BTN2A1 agonist increases the percentage of γδ T cells expressing CD25 on the cell surface to at least 10% of the cells in a population of γδ T cells. In one example, the BTN2A1 agonist increases the percentage of γδ T cells expressing CD25 on the cell surface to at least 15% of the cells in a population of γδ T cells. In one example, the BTN2A1 agonist increases the percentage of γδ T cells expressing CD25 on the cell surface to at least 20% of the cells in a population of γδ T cells. In one example, the BTN2A1 agonist increases the percentage of γδ T cells expressing CD25 on the cell surface to at least 30% of the cells in a population of γδ T cells. In one example, the BTN2A1 agonist increases the percentage of γδ T cells expressing CD25 on the cell surface to at least 40% of the cells in a population of γδ T cells.

In another example, the BTN2A1 agonist increases secretion of interferon-γ by γδ T cells as measured in an assay comprising culturing a population of γδ T cells in an in vitro cell culture with the BTN2A1 agonist for a period of at least 6 hours or 8 hours or 10 hours or 12 hours and measuring the amount of interferon-γ per mL of cell culture fluid.

In one example, the BTN2A1 agonist increases secretion of interferon-γ to 10 μg/mL of fluid from a γδ T cell culture. In one example, the BTN2A1 agonist increases secretion of interferon-γ to 20 μg/mL of fluid from a γδ T cell culture. In one example, the BTN2A1 agonist increases secretion of interferon-γ to 30 μg/mL of fluid from a γδ T cell culture. In one example, the BTN2A1 agonist increases secretion of interferon-γ to 40 μg/mL of fluid from a γδ T cell culture.

In one example, the agonist increases secretion of one or more additional or alternative cytokines (additional to or alternative to interferon-γ).

In a further example, induces γδ T cells to kill and/or inhibit the growth of cells (e.g., cancer cells or infected cells) as measured in an assay comprising culturing cells, e.g., melanoma cells or a melanoma cell line, in the presence of γδ T cells and a BTN2A1 agonist and a reagent that is reduced by living cells (e.g., 3-(4,5-dimethylthiazol-2-yl)-2,5-diphenyltetrazolium bromide [MTT]) to a detectable reagent (e.g., formazan) and detecting the detectable reagent, wherein a reduced level of the detectable reagent in the presence of the BTN2A1 agonist compared to in the absence of the BTN2A1 agonist indicates that the cells have been killed or the growth of the cells has been inhibited.

In one example, the BTN2A1 agonist is a protein comprising an antigen binding domain.

In an embodiment, the protein is:

(i) a single chain Fv fragment (scFv);

(ii) a dimeric scFv;

(iii) a Fv fragment;

(iv) a single domain antibody(sdAb)

(v) a diabody, triabody, tetrabody or higher order multimer;

(vi) Fab fragment;

(vii) a Fab′ fragment;

(viii) a F(ab′) fragment;

(ix) a F(ab′)₂ fragment;

(x) any one of (i)-(ix) linked to a Fc region of an antibody;

(xi) any one of (i)-(ix) fused to an antibody or antigen binding fragment thereof that binds to an immune effector cell; or

(xii) an antibody.

In one example, the BTN2A1 agonist is an antibody comprising a light chain variable region (V_{L}) comprising a sequence set forth in SEQ ID NO: 140 and a heavy chain variable region (V_{H}) comprising a sequence set forth in SEQ ID NO: 144.

In one example, the BTN2A1 agonist is an antibody comprising a V_{L} comprising a sequence set forth in SEQ ID NO: 148 and a V_{H} comprising a sequence set forth in SEQ ID NO: 152.

In one example, the BTN2A1 agonist is an antibody comprising a V_{L} comprising a sequence set forth in SEQ ID NO: 156 and a V_{H} comprising a sequence set forth in SEQ ID NO: 160.

In one example, the BTN2A1 agonist is an antibody comprising a V_{L} and a V_{H} comprising the CDRs of any of the foregoing antibodies. For example, the CDRs are as defined by the numbering system of Kabat (Kabat Sequences of Proteins of Immunological Interest, National Institutes of Health, Bethesda, Md., 1987 and 1991).

For example, the BTN2A1 agonist is an antibody comprising:

(i) a V_{L} comprising:

(a) a CDR1 comprising a sequence set forth in amino acids 26-33 of SEQ ID NO: 140;

(b) a CDR2 comprising a sequence set forth in amino acids 51-53 of SEQ ID NO: 140; and

(c) a CDR3 comprising a sequence set forth in amino acids 90-98 of SEQ ID NO: 140; and/or

(ii) a V_{H} comprising:

(a) a CDR1 comprising a sequence set forth in amino acids 26-33 of SEQ ID NO: 144;

(b) a CDR2 comprising a sequence set forth in amino acids 51-58 of SEQ ID NO: 144; and

(c) a CDR3 comprising a sequence set forth in amino acids 97-109 of SEQ ID NO: 144.

For example, the BTN2A1 agonist is an antibody comprising:

(i) a V_{L} comprising:

(a) a CDR1 comprising a sequence set forth in amino acids 26-33 of SEQ ID NO: 148;

(b) a CDR2 comprising a sequence set forth in amino acids 51-53 of SEQ ID NO: 148; and

(c) a CDR3 comprising a sequence set forth in amino acids 90-100 of SEQ ID NO: 148; and/or

(ii) a V_{H} comprising:

(a) a CDR1 comprising a sequence set forth in amino acids 26-33 of SEQ ID NO: 152;

(b) a CDR2 comprising a sequence set forth in amino acids 51-58 of SEQ ID NO: 152; and

(c) a CDR3 comprising a sequence set forth in amino acids 97-116 of SEQ ID NO: 152.

For example, the BTN2A1 agonist is an antibody comprising:

(i) a V_{L} comprising:

(a) a CDR1 comprising a sequence set forth in amino acids 26-33 of SEQ ID NO: 156;

(b) a CDR2 comprising a sequence set forth in amino acids 51-53 of SEQ ID NO: 156; and

(c) a CDR3 comprising a sequence set forth in amino acids 90-100 of SEQ ID NO: 156; and/or

(ii) a V_{H} comprising:

(a) a CDR1 comprising a sequence set forth in amino acids 26-33 of SEQ ID NO: 160;

(b) a CDR2 comprising a sequence set forth in amino acids 51-58 of SEQ ID NO: 160; and

(c) a CDR3 comprising a sequence set forth in amino acids 97-109 of SEQ ID NO: 160.

In one example, the BTN2A1 agonist is an antibody comprising:

(i) a V_{L} comprising:

(a) a CDR1 comprising a sequence set forth in SEQ ID NO: 141;

(b) a CDR2 comprising a sequence set forth in SEQ ID NO: 142; and

(c) a CDR3 comprising a sequence set forth in SEQ ID NO: 143; and/or

(ii) a V_{H} comprising:

(a) a CDR1 comprising a sequence as set forth in SEQ ID NO:145;

(b) a CDR2 comprising a sequence as set forth in SEQ ID NO: 146; and

(c) a CDR3 comprising a sequence as set forth in SEQ ID NO: 147.

In one example, the BTN2A1 agonist is an antibody comprising:

(i) a V_{L} comprising:

(a) a CDR1 comprising a sequence set forth in SEQ ID NO: 149;

(b) a CDR2 comprising a sequence set forth in SEQ ID NO: 150; and

(c) a CDR3 comprising a sequence set forth in SEQ ID NO: 151; and/or

(ii) a V_{H} comprising:

(a) a CDR1 comprising a sequence as set forth in SEQ ID NO:153;

(b) a CDR2 comprising a sequence as set forth in SEQ ID NO: 154; and

(c) a CDR3 comprising a sequence as set forth in SEQ ID NO: 155.

In one example, the BTN2A1 agonist is an antibody comprising:

(i) a V_{L} comprising:

(a) a CDR1 comprising a sequence set forth in SEQ ID NO: 157;

(b) a CDR2 comprising a sequence set forth in SEQ ID NO: 158; and

(c) a CDR3 comprising a sequence set forth in SEQ ID NO: 159; and/or

(ii) a V_{H} comprising:

(a) a CDR1 comprising a sequence as set forth in SEQ ID NO:161;

(b) a CDR2 comprising a sequence as set forth in SEQ ID NO: 162; and

(c) a CDR3 comprising a sequence as set forth in SEQ ID NO: 163.

In one example, the BTN2A1 agonist of the present disclosure is an affinity matured, chimeric, CDR grafted, or humanized antibody, or antigen binding fragment thereof.

In one example, the BTN2A1 agonist is a protein, for example, an antibody comprising a variable region that competitively inhibits the binding of an antibody disclosed herein and/or that binds to the same epitope as an antibody disclosed herein.

The present disclosure also provides a method for activating γδ T cells that express a Vγ9+ TCR in a subject, the method comprising administering a BTN2A1 agonist as described above to the subject.

The present disclosure also provides a method of inducing or enhancing Vγ9+ γδ T cell responses in a subject, wherein the method comprises administering a BTN2A1 agonist as described above to the subject.

The present disclosure also provides a method for activating γδ T cells that express a Vγ9+ TCR in vitro or ex vivo, the method comprising culturing the γδ T cells and cells expressing BTN2A1 in the presence of a BTN2A1 agonist as described above. In an embodiment, the method further comprises the step of administering the activated γδ T cells to a subject in need thereof.

The present disclosure also provides a method of preventing, treating, delaying the progression of, preventing a relapse of, or alleviating a symptom of an autoimmune disease, transplantation rejection, graft versus host disease, or graft versus tumour effect, the method comprising administering a BTN2A1 agonist as described above to a subject in need thereof in an amount sufficient to prevent, treat, delay the progression of, prevent a relapse of, or alleviate the symptom of the autoimmune disease, transplant rejection, graft versus host disease, or graft versus tumour effect in the subject.

The present disclosure also provides a method of preventing, treating, delaying the progression of, preventing a relapse of, or alleviating a symptom of a cancer or an infection, the method comprising administering a BTN2A1 agonist as described above to a subject in need thereof in an amount sufficient to prevent, treat, delay the progression of, prevent a relapse of, or alleviate the symptom of the cancer or infection in the subject.

### Key to Sequence Listing

SEQ ID NO: 1 is an amino acid sequence of human BTN2A1 isoform 1.
SEQ ID NO: 2 is an amino acid sequence of human BTN2A1 isoform 2.
SEQ ID NO: 3 is an amino acid sequence of human BTN2A1 isoform 3.
SEQ ID NO: 4 is an amino acid sequence of human BTN2A1 isoform 4.
SEQ ID NO: 5 is an amino acid sequence of human Annexin A5.
SEQ ID NO: 6 is an amino acid sequence of human Annexin A1.
SEQ ID NO: 7 is an amino acid sequence of a Lactadherin C1C2 domain.
SEQ ID NO: 8 is an amino acid sequence of PSP1 protein.
SEQ ID NOs: 9-69 are nucleotide sequences encoding primers (see Table 2).
SEQ ID NO: 70 is an amino acid sequence of δ2 (clone 6).
SEQ ID NO: 71 is an amino acid sequence of δ2 (clone 3).
SEQ ID NO: 72 is an amino acid sequence of δ2 (clone 4).
SEQ ID NO: 73 is an amino acid sequence of δ2 (clone 5).
SEQ ID NO: 74 is an amino acid sequence of δ2 (clone 7).
SEQ ID NO: 75 is an amino acid sequence of variable region of δ2 (clone 6).
SEQ ID NO: 76 is an amino acid sequence of variable region of δ2 (clone 3).
SEQ ID NO: 77 is an amino acid sequence of variable region of δ2 (clone 4).
SEQ ID NO: 78 is an amino acid sequence of variable region of δ2 (clone 5).
SEQ ID NO: 79 is an amino acid sequence of variable region of δ2 (clone 7).
SEQ ID NO: 80 is an amino acid sequence of CDR3δ (clone 3)
SEQ ID NO: 81 is an amino acid sequence of CDR3δ (clone 4)
SEQ ID NO: 82 is an amino acid sequence of CDR3δ (clone 5)
SEQ ID NO: 83 is an amino acid sequence of CDR3δ (clone 6)
SEQ ID NO: 84 is an amino acid sequence of CDR3δ (clone 7)
SEQ ID NO: 85 is an amino acid sequence of γ9 (clone 6).
SEQ ID NO: 86 is an amino acid sequence of γ9 (clone 3).
SEQ ID NO: 87 is an amino acid sequence of γ9 (clone 4).
SEQ ID NO: 88 is an amino acid sequence of γ9 (clone 5).
SEQ ID NO: 89 is an amino acid sequence of γ9 (clone 7).
SEQ ID NO: 90 is an amino acid sequence of variable region of 19 (clone 6).
SEQ ID NO: 91 is an amino acid sequence of variable region of γ9 (clone 3).
SEQ ID NO: 92 is an amino acid sequence of variable region of γ9 (clone 4).
SEQ ID NO: 93 is an amino acid sequence of variable region of γ9 (clone 5).
SEQ ID NO: 94 is an amino acid sequence of variable region of γ9 (clone 7).
SEQ ID NO: 95 is an amino acid sequence of CDR3γ (clone 3)
SEQ ID NO: 96 is an amino acid sequence of CDR3γ (clone 4)
SEQ ID NO: 97 is an amino acid sequence of CDR3γ (clone 5)
SEQ ID NO: 98 is an amino acid sequence of CDR3γ (clone 6)
SEQ ID NO: 99 is an amino acid sequence of CDR3γ (clone 7)
SEQ ID NO: 100 is an amino acid sequence of Hu34C V_{H}
SEQ ID NO: 101 is an amino acid sequence of Hu34C V_{L}
SEQ ID NO: 102 is an amino acid sequence of Hu34C V_{H} CDR1
SEQ ID NO: 103 is an amino acid sequence of Hu34C V_{H} CDR2
SEQ ID NO: 104 is an amino acid sequence of Hu34C V_{H} CDR3
SEQ ID NO: 105 is an amino acid sequence of Hu34C V_{L} CDR1
SEQ ID NO: 106 is an amino acid sequence of Hu34C V_{L} CDR2
SEQ ID NO: 107 is an amino acid sequence of Hu34C V_{L} CDR3
SEQ ID NO: 108 is an amino acid sequence of clone 227 V_{H}
SEQ ID NO: 109 is an amino acid sequence of clone 227 V_{L}
SEQ ID NO: 110 is an amino acid sequence of clone 227 V_{H} CDR1
SEQ ID NO: 111 is an amino acid sequence of clone 227 V_{H} CDR2
SEQ ID NO: 112 is an amino acid sequence of clone 227 V_{H} CDR3
SEQ ID NO: 113 is an amino acid sequence of clone 227 V_{L} CDR1
SEQ ID NO: 114 is an amino acid sequence of clone 227 V_{L} CDR2
SEQ ID NO: 115 is an amino acid sequence of clone 227 V_{L} CDR3
SEQ ID NO: 116 is an amino acid sequence of clone 236 V_{H}
SEQ ID NO: 117 is an amino acid sequence of clone 236 V_{L}
SEQ ID NO: 118 is an amino acid sequence of clone 236 V_{H} CDR1
SEQ ID NO: 119 is an amino acid sequence of clone 236 V_{H} CDR2
SEQ ID NO: 120 is an amino acid sequence of clone 236 V_{H} CDR3
SEQ ID NO: 121 is an amino acid sequence of clone 236 V_{L} CDR1
SEQ ID NO: 122 is an amino acid sequence of clone 236 V_{L} CDR2
SEQ ID NO: 123 is an amino acid sequence of clone 236 V_{L} CDR3
SEQ ID NO: 124 is an amino acid sequence of clone 266 V_{H}
SEQ ID NO: 125 is an amino acid sequence of clone 266 V_{L}
SEQ ID NO: 126 is an amino acid sequence of clone 266 V_{H} CDR1
SEQ ID NO: 127 is an amino acid sequence of clone 266 V_{H} CDR2
SEQ ID NO: 128 is an amino acid sequence of clone 266 V_{H} CDR3
SEQ ID NO: 129 is an amino acid sequence of clone 266 V_{L} CDR1
SEQ ID NO: 130 is an amino acid sequence of clone 266 V_{L} CDR2
SEQ ID NO: 131 is an amino acid sequence of clone 266 V_{L} CDR3
SEQ ID NO: 132 is an amino acid sequence of clone 267 V_{H}
SEQ ID NO: 133 is an amino acid sequence of clone 267 V_{L}
SEQ ID NO: 134 is an amino acid sequence of clone 267 V_{H} CDR1
SEQ ID NO: 135 is an amino acid sequence of clone 267 V_{H} CDR2
SEQ ID NO: 136 is an amino acid sequence of clone 267 V_{H} CDR3
SEQ ID NO: 137 is an amino acid sequence of clone 267 V_{L} CDR1
SEQ ID NO: 138 is an amino acid sequence of clone 267 V_{L} CDR2
SEQ ID NO: 139 is an amino acid sequence of clone 267 V_{L} CDR3
SEQ ID NO: 140 is an amino acid sequence of the V_{L} of antibody 244
SEQ ID NO: 141 is an amino acid sequence of CDR1 of the V_{L} of antibody 244
SEQ ID NO: 142 is an amino acid sequence of CDR2 of the V_{L} of antibody 244
SEQ ID NO: 143 is an amino acid sequence of CDR3 of the V_{L} of antibody 244
SEQ ID NO: 144 is an amino acid sequence of the V_{H} of antibody 244
SEQ ID NO: 145 is an amino acid sequence of CDR1 of the V_{H} of antibody 244
SEQ ID NO: 146 is an amino acid sequence of CDR2 of the V_{H} of antibody 244
SEQ ID NO: 147 is an amino acid sequence of CDR3 of the V_{H} of antibody 244
SEQ ID NO: 148 is an amino acid sequence of the V_{L} of antibody 253
SEQ ID NO: 149 is an amino acid sequence of CDR1 of the V_{L} of antibody 253
SEQ ID NO: 150 is an amino acid sequence of CDR2 of the V_{L} of antibody 253
SEQ ID NO: 151 is an amino acid sequence of CDR3 of the V_{L} of antibody 253
SEQ ID NO: 152 is an amino acid sequence of the V_{H} of antibody 253
SEQ ID NO: 153 is an amino acid sequence of CDR1 of the V_{H} of antibody 253
SEQ ID NO: 154 is an amino acid sequence of CDR2 of the V_{H} of antibody 253
SEQ ID NO: 155 is an amino acid sequence of CDR3 of the V_{H} of antibody 253
SEQ ID NO: 156 is an amino acid sequence of the V_{L} of antibody 259
SEQ ID NO: 157 is an amino acid sequence of CDR1 of the V_{L} of antibody 259
SEQ ID NO: 158 is an amino acid sequence of CDR2 of the V_{L} of antibody 259
SEQ ID NO: 159 is an amino acid sequence of CDR3 of the V_{L} of antibody 259
SEQ ID NO: 160 is an amino acid sequence of the V_{H} of antibody 259
SEQ ID NO: 161 is an amino acid sequence of CDR1 of the V_{H} of antibody 259
SEQ ID NO: 162 is an amino acid sequence of CDR2 of the V_{H} of antibody 259
SEQ ID NO: 163 is an amino acid sequence of CDR3 of the V_{H} of antibody 259

### BRIEF DESCRIPTION OF DRAWINGS

FIG. 1. Vγ9Vδ2⁺ γδ T cell receptor tetramer staining is dependent on BTN2A1. (A) Vγ9Vδ2⁺ γδTCR tetramer staining of various cell lines. Histograms depict γδTCR tetramers #3-#7; irrelevant control (mouse CD1d-α-GalCer) tetramer; streptavidin (SAv)-PE control. (B) Volcano plot depicting log₂ (fold-change) versus −log₁₀ (p-value) for each gRNA, between unsorted and Vγ9Vδ2 γγTCR tetramer^{lo} LM-MEL-62 cells, where dark gray depicts significant differences (false discovery rate <0.05). (C) Vγ9Vδ2⁺ γδTCR tetramer staining of LM-MEL-62 BTN2A1^{null} and LM-MEL-75 BTN2A1^{null} cells compared to parental cells. (D) Anti-BTN2A1 mAb (clone 231), anti-BTN3A1/3A2/3A3 mAb (clone 103.2), and Vγ9Vδ2⁺ γδTCR tetramer (#6) staining on parental and BTN2A1^{null1/null2} LM-MEL-62 cells transfected with either BTN2A1 or BTN3A1. *γδTCR tetramer staining of WT cells is depicted twice. (E) Vγ9Vδ2⁺ γδTCR tetramer #6 staining of LM-MEL-62, LM-MEL-75 and HEK-293T cells, following pre-incubation of cells with a panel of anti-BTN2A1 mAb, compared to isotype control (white). Lower histograms depict control staining with irrelevant mouse CD1d-α-GalCer tetramer. tet, tetramer. Data in (A), (C), (D), (E) are representative of two independent experiments.

FIG. 2. BTN2A1 binds Vγ9⁺ γδ T cell receptors. (A) BTN2A1 tetramer-PE (first column) or streptavidin-PE control (second column) versus CD3ε staining on three representative human PBMC samples. Histograms depict BTN2A1 tetramer-PE staining (white) or streptavidin-PE control (gray) on gated γδ T cell (CD3⁺ γδTCR⁺), αβ T cell (CD3⁺ γδTCR⁻), B cell (CD3⁻ CD19⁺), monocyte (CD3⁻ CD19⁻ CD14⁺) or other (CD3⁻ CD19⁻ CD14⁻) subsets. Box and whisker plots (right) depict the percentage of each cell lineage that binds to BTN2A1 tetramer in blood samples from different donors. (B) BTN2A1 tetramer (white histograms) overlaid with streptavidin-PE alone control (gray histograms) staining, on Vγ9⁺ Vδ2⁺, Vγ9⁺Vδ1⁺, Vγ9⁻Vδ1⁺ γδ T cells, with parent gating shown to the left. Box and whisker plots (right) depict the percentage of each γδ T cell subset that binds to BTN2A1 tetramer-PE in different donors. (C) FRET fluorescence (histogram overlays) between BTN2A1 tetramer-PE and CD3ε-APC on dual stained or single-stained controls using purified in vitro-expanded Vδ2⁺ T cells. Box and whisker plots depict FRET mean fluorescence intensity (MFI) in γδ T cell subsets from different human donors. (D) Binding of soluble BTN2A1 (200-3.1 μM) to immobilized Vγ9⁺ Vδ2⁺ (‘TCR #6’, left), Vγ9⁺ Vδ1⁺ (‘hybrid’, middle) and Vγ5⁺ Vδ1⁺ (‘9C2’, right) γδTCRs, as measured by surface plasmon resonance. Saturation plots (below) depict binding at equilibrium and Scatchard plots. K_{D}, dissociation constant at equilibrium±SEM; SAv, streptavidin. Data in (A) represent n=8 donors pooled from two independent experiments; (B) n=8 donors from two experiments; (C) n=7 donors pooled from three independent experiments; (D) n=2 separate experiments, one of which (Expt 2) was performed in duplicate and averaged.

FIG. 3. γδ T cell functional responses to pAg depend on BTN2A1. (A) CD25 expression and CD3ε mean fluorescence intensity (MFI) on Vδ2⁺ and control Vδ1⁺ T cells gated among PBMCs cultured for 24 h±4 μM zoledronate and ±10 μg/ml neutralizing anti-BTN2A1 mAb as indicated. *, p<0.05; **, p<0.01, ***, p<0.001, by ANOVA. (B) IFN-γ and TNF concentration in the culture supernatants from (A). **, p<0.01; ***, p<0.001, by Friedman test. (C) CD3 MFI and CD25 expression on purified in vitro-expanded Vδ2⁺ T cells co-cultured with parental or BTN2A1^{null} LM-MEL-62 APCs without (gray) or with (dark gray) 4 μM zoledronate. Each symbol represents a different donor. Bar graphs depict mean±SEM. (D) Number of Vδ2⁺ γδ T cells in co-cultures of PBMC with parental or BTN2A1^{null} LM-MEL-62 APC after a 2 day challenge with 1 μM zoledronate followed by maintenance of non-adherent PBMC for an additional 7 d in media containing IL-2. *, p<0.05 using a Mann-Whitney test. (E) Cell viability (mean±SEM) as determined using the metabolic dye MTS, normalized against input cell number, of co-cultures of parental or BTN2A1^{null} LM-MEL-62 targets with in vitro-expanded Vδ2⁺ T cells, at the indicated time points ±1 μM zoledronate. *, p<0.05 using a Mann-Whitney test. (F) CD25 expression (left) and IFN-γ concentration (right) following culture of purified in vitro-expanded Vδ2⁺ T cells with HMBPP (0.5 ng/ml) or plate-bound anti-CD3 plus anti-CD28 (10 μg/ml each)±10 μg/ml neutralizing anti-BTN2A1 mAb. Data in (A) and (B) n=8 donors pooled from two independent experiments; (C) n=3 donors pooled from three independent experiments, each performed with n=4 technical replicates indicated by different symbols; (D) n=4 donors, each averaged from 1-5 technical replicates across five independent experiments; (E) n=8 donors pooled from two independent experiments; (F) n=4 donors, each averaged from 2-6 technical replicates across six independent experiments. Zol, zoledronate.

FIG. 4. BTN2A1 and BTN3A1 are both necessary for pAg presentation. (A) CD69 expression on G115 Vγ9Vδ2⁺ γδ TCR (top row), 9C2 Vγ5Vδ1⁺ γδ TCR (middle), and parental (TCR⁻) J.RT3-T3.5 (bottom row) Jurkat cells after overnight co-culture with the indicated APCs, in the presence (dark gray) or absence (gray) of 40 μM zoledronate. Numbers indicate the median fluorescence intensity. (B) Change in CD25 expression (normalized to unstimulated control for each sample) on purified in vitro-expanded γδ T cells co-cultured for 24 h in the presence (dark gray) or absence (gray) of 4 μM zoledronate with CHO-K1 (hamster origin) or NIH-3T3 (mouse origin) APCs transfected with the indicated combinations of (B) B7NL3, B7NL8, BTN2A1, BTN3A1 and BTN3A2, or (C) BTN2A1ΔB30, BTN3A1 and BTN3A2. (D) γδ T cells co-cultured as in (A), except in the presence of a 1:1 mixture of two populations of APCs, each transfected separately with combinations of BTN2A1, BTN3A1 and BTN3A2. Each symbol and connecting line represents a different donor. *, p<0.05; **, p<0.01 using a Wilcoxon paired test. Bar graphs depict mean±SEM. Data in (A) representative of one of three similar experiments; (B-D) represents n=7-9 donors per group pooled from 3-5 independent experiments.

FIG. 5. BTN2A1 associates with BTN3A1 on the cell surface. (A) Z-stack confocal microscopy of surface BTN2A1 (clone 259) and BTN3A (clone 103.2), and pan-HLA class I (clone W6/32) on parental LM-MEL-75 (“WT”, top row), BTN2A1^{null} (middle row) and BTN3A1^{null} (bottom row) cells. (B) Graph depicts Pearson correlation coefficients for individual fields of view. Representative voxel density plots depicting correlation between anti-BTN2A1 versus anti-BTN3A1/3A2/3A3 (“BTN3A”) (left), anti-BTN2A1 versus anti-HLA-A,B,C (middle), and anti-BTN3A versus anti-HLA-A,B,C (right). ***, p<0.001 using a Kruskal-Wallis with Dunn's post test. (C) Anti-BTN2A1 versus BTN3A co-staining, or single staining, or mouse IgG1 versus mouse IgG2a isotype control staining (x- and y-axis respectively) on LM-MEL-75 cells using the indicated mAb clones (top row). Histograms (second row) depict FRET fluorescence. (D) Percentage of FRET⁺ cells between butyrophilin^{CFP/YFP}-transfected NIH-3T3 cells. Data are representative of (A) and (B) two pooled independent experiments; (C) one experiment; (D) four independent experiments.

FIG. 6. Vγ9⁺Vδ2⁺γδ T cell receptors contain two distinct ligand-binding domains. (A) BTN2A1 tetramer-PE (dark gray) and control streptavidin-PE alone (black) staining of gated GFP⁺CD3⁺ HEK-293T cells transfected with single residue G115 γδTCR alanine mutants (or control Jurkat.9C2 γδTCR), normalized to BTN2A1 tetramer staining of G115 WT γδTCR. (B) Cartoon view of the G115 γδTCR (pdb code 1HXM (T. J. Allison et al. (2001)) Vγ9 ABED β-sheet depicting the side chains of R20, E70, and H85. (C) CD69 expression on Jurkat cells expressing G115 γδTCR alanine mutants (or 9C2 γδTCR⁺ or parental γδTCR⁻ Jurkat cells), normalized to the activation levels of G115 WT γδTCR⁺ Jurkat cells, after overnight culture with LM-MEL-75 APCs in the presence (dark gray) or absence (black) of 40 μM zoledronate. (D) Surface of G115 γδTCR (pdb code 1HXM (25)) depicting the residues important for BTN2A1 tetramer binding (top row) and zoledronate reactivity (bottom row). Side chains of residues with >75% loss of BTN2A1 binding or CD69 induction are labelled and also shown in dark gray; 50%-75% reduction are labelled and also shown in medium-dark gray; <50% reduction grey; Vδ2, light gray; Vγ9, medium gray; constant regions, white. MFI, median fluorescence intensity; SAv, streptavidin alone control; unstim, unstimulated control. Data in (A) and (B) represent the mean±SEM of N=3 separate experiments.

FIG. 7. Agonistic activity of anti-BTN3A1 mAb clone 20.1 depends on BTN2A1. CD69 expression on Jurkat cells expressing Vγ9Vδ2⁺ γδTCR (clone G115), or the indicated G115 γδTCR mutants, or control Vγ5Vδ1⁺ γδTCR (clone 9C2) following co-culture with either parental LM-MEL-75 (“WT”) or BTN2A1^{null} APCs pre-incubated with anti-BTN3A (clone 20.1, 10 μg/ml, dark gray histograms) or isotype control (mouse IgG1, 10 μg/ml, light gray). Data representative of one of two separate experiments.

FIG. 8. Generation of soluble Vγ9Vδ2⁺ γδ TCR tetramers. (A) PCR for Vδ2 and Vγ9 on single cell-sorted Vδ2⁺ γδ T cells from PBMCs. Negative controls depict PCR on empty wells from the same plate. (B) Paired γ-chain and δ-chain gene usage and CDR3 motifs from selected cells. (C) Soluble γδ TCR construct design containing full-length ectodomains coupled to leucine zippers and an Avi-tag/His6 tag. (D) SDS-PAGE analysis of denatured soluble biotinylated and unbiotinylated Vγ9Vδ2⁺ γδ TCR, either alone or mixed with undenatured native streptavidin (SAv), showing incorporation of the biotinylated TCR δ-chain into a complex with native streptavidin. MW, molecular weight markers.

FIG. 9. Identification of Vγ9Vδ2⁺ γδ TCR ligands using a whole genome CRISPR/Cas9 knockout screen. (A) γδTCR tetramer #6^{lo} LM-MEL-62 cells were sort-purified four consecutive times, from n=4 separate replicates. Histograms depict γδTCR tetramer #6 overlaid with control staining after 1-2 weeks culture following each round of sorting. (B) Top forty guide RNA gene targets within the γδTCR tetramer #6^{lo} population, compared to control unsorted (“pre-sort”) LM-MEL-62 cells.

FIG. 10. Generation of BTN2A1 and BTN3A1 knockout cell lines.

BTN2A1^{null} and BTN3A1^{null} LM-MEL-62 or LM-MEL-75 cells were generated via transient transfection of target cells with vectors encoding Cas9 and specific guide RNA, followed by bulk cell sorting. (A) Anti-BTN2A1 (clone 231) and anti-BTN3A1/3A2/3A3 (clone 103.2) staining of each cell line overlaid with isotype controls. (B) Vγ9Vδ2⁺ γδ TCR tetramer #6 staining of each cell line (dark gray) overlaid with irrelevant tetramer control (mouse CD1d-α-GalCer, gray). Data are representative of two similar experiments.

FIG. 11. Generation of anti-BTN2A1 mAb. (A) Alignment of BTN2A1, BTN2A2, BTN3A1, BTN3A2 ectodomains. (B) Binding of anti-BTN2A1 mAb clones to plate-bound BTN2A1, BTN2A2, or BTN3A3 ectodomains by ELISA, where heat maps depict absorbance. (C) Anti-BTN2A1 mAb reactivity to mouse NIH-3T3 cells transfected with full length human BTN2A1, BTN2A2, or BTN3A1, or untransfected cells, as indicated. Data averaged from N=2 separate experiments. (D) Reactivity of selected anti-BTN2A1 clones or isotype controls (mouse IgG2a κ, clone BM4) to LM-MEL-62 parental (“WT”), BTN2A1^{null1} and BTN2A1^{null2} cells, using a BV421-conjugated secondary polyclonal Ab. The same isotype control is overlaid across each individual row. (E) Reactivity of selected anti-BTN2A1 clones to LM-MEL-62 parental (“WT”), BTN2A1^{null} and BTN3A1^{null} cells using a PE-conjugated secondary polyclonal Ab. A450, absorbance at 450 nm.

FIG. 12. Generation of BTN2A1 tetramers. (A) Construct design including BTN2A1 ectodomain (IgV and IgC domains; Gln29 to Ser245) fused to a C-terminal linker (amino acid sequence: GTGSGSGG), followed by Avi (biotin ligase)- and His6-tags (amino acid sequence: LNDIFEAQKIEWHEHHHHH). (B) SDS-PAGE analysis of biotinylated BTN2A1 (and control BTN3A1) ectodomains produced in 293T cells. Right-hand lane denatured BTN2A1-biotin complexed with undenatured streptavidin (SAv.). (C) ELISA of plate-bound BTN2A1 ectodomain reactivity to anti-BTN2A1 clones Hu34C and 231 compared to isotype control (clone BM4). Data in panel (C) representative of one experiment. MW, molecular weight markers.

FIG. 13. BTN2A1 is specifically recognized by Vγ9Vδ2⁺ γδ TCR tetramers.

Vγ9Vδ2⁺ γδ TCR tetramer #6, irrelevant control tetramer (mouse CD1d-α-GalC), or control streptavidin (SAv.) alone staining on gated GFP⁺ mouse 3T3 cells following transfection with either human BTN2A1, BTN2A2, BTNL3 plus BTNL8, or BTN3A1 plus BTN3A2 (parent gating is depicted in the top row of density plots). Data are representative of two similar experiments.

FIG. 14. Antagonist anti-BTN2A1 mAb specifically block pAg-mediated activation of Vδ2⁺ γδ T cells but not peptide-mediated activation of CD8⁺ αβ T cells.

(A) Intracellular IFN-γ expression on gated Vδ2⁺ CD3⁺ T cells (left) or CD8⁺ CD3⁺ T cells (right) amongst PBMCs following in vitro challenge with either the pAg HMBPP (0.5 ng/ml) or zoledronate (4 μM) alone or in combination with CEF peptide mixture containing immunogenic peptides derived from cytomegalovirus, Epstein-Barr virus and influenza (1 μg/ml)±10 μg/ml neutralizing anti-BTN2A1 mAbs (clones Hu34C, 236, 259, 267), anti-BTN3A molecules (clone 103.2) or isotype control (mouse IgG2a, κ, clone BM4). (B) Representative gating (top row) and plots of IFN-γ staining on gated Vδ2⁺CD3⁺ T cells (middle row), or CD8⁺ CD3⁺ T cells (bottom row). Data are representative of seven donors from two independent experiments.

FIG. 15. Jurkat G115 Vγ9Vδ2⁺ γδ T cell responses to zoledronate, HMBPP and IPP depend on BTN2A1.

(A) CD69 induction on either Jurkat G115 Vγ9Vδ2 γδTCR⁺ or control Jurkat 9C2 Vγ5Vδ1 γδTCR⁺ T cells following coculture with graded doses of the pAgs HMBPP, IPP, or zoledronate ±parental LM-MEL-75 APCs. (B) representative CD69 histograms and (C) expression levels following coculture of Jurkat G115 and Jurkat 9C2 T cell lines with either parental LM-MEL-75, BTN2A1^{null} or BTN3A1^{null} APCs±HMBPP (100 nM), IPP (100 μM) or zoledronate (40 μM). Data in (A) from one experiment; (B) and (C) pooled from N=4 independent experiments.

FIG. 16. BTN2A1 plus BTN3A1 engender mouse APCs with the capacity to present pAg to γδ T cells. (A) BTN2A1 (clone 231) versus BTN3A1/3A2/3A3 staining (clone 103.2), or isotype control staining (mouse IgG2a clone BM4), on NIH-3T3 cells transfected with the indicated combinations of B7NL3, BTNL8, BTN2A, BTN3A1 and BTN3A2, or BTN2A1ΔB30. (B) CD25 expression on purified in vitro-expanded γδ T cells co-cultured for 24 h in the presence (dark gray) or absence (gray) of 4 μM zoledronate with CHO-K1 or NIH-3T3 APCs transfected with the indicated combinations of B7NL3, BTNL8, BTN2A, BTN3A1 and BTN3A2, or BTN2A1ΔB30. Three groups on the right depict γδ T cells co-cultured in the presence of a 1:1 mixture of 2 populations of APCs, each transfected separately with the indicated combinations of BTN2A, BTN3A1 and BTN3A2. (C) Schematic of BTN2A1 and BTN2A1ΔB30 structures (left), and histograms depicting anti-BTN2A1 (clone 259) and γδTCR tetramer (#6) on NIH-3T3 cells transfected with BTN2A1 or BTN2A1ΔB30, overlaid with relevant controls. Data represent n=7-9 donors per group pooled from 3-5 independent experiments. TM, transmembrane domain.

FIG. 17. No detectable binding of HMBPP to intracellular B30.2 domain of BTN2A1. (A) Raw isothermal titration calorimetry traces and (B) binding isotherms of recombinant BTN2A1 (left column) or BTN3A1 (right column) B30.2 domains (100 μM), upon serial injections of the pAgs HMBPP, IPP, or PBS buffer alone. Data shown from one of two independent experiments.

FIG. 18 Association between BTN2A1 and BTN3A1 on the cell surface is independent of intracellular B30.2 domains.

Contour plots (top row) depict BTN2A1 (clone 259) versus BTN3A (clone 103.2) staining (dark gray), overlaid with isotype control staining (mouse IgG1 clone MOPC-173 on the x-axis versus mouse IgG2a clone BM4 on the y-axis versus, gray) on mouse NIH-3T3 cells transfected with the indicated combinations of BTN2A1, BTN3A1, BTN3A1 and/or BTN2A1ΔB30. Histograms (second row) depict FRET signal in each staining condition. Data representative of 2 independent experiments.

FIG. 19. Generation of CFP- and YFP-tagged butyrophilin constructs.

(A) Design of full length BTN2A1, BTN3A1, B7NL3, and BTNL8 with either a “long” or “short” C-terminal flexible linker coupled to CFP or YFP. (B) Amino acid sequences of C-terminal linkers and CFP/YFP domains. (C) Representative plots depicting anti-BTN2A1 (clone 231) and anti-BTN3A molecules (clone 103.2) mAb staining (dark gray) or isotype control staining (IgG1 versus IgG2a, black) on mouse NIH-3T3 cells transiently transfected with each respective construct. (D) Representative plots depicting BTN2A1 (left) and BTN3A1 (right) surface expression on mouse NIH-3T3 cells transfected with WT BTN molecules, or CFP/YFP-tagged BTN molecules.

FIG. 20. Intracellular domains of BTN2A1 and BTN3A1 are associated and this is not affected by pAg.

(A) Plots depict FRET versus donor fluorophore (CFP) on mouse 3T3 cells transfected with different combinations of butyrophilin molecules (top row) or single-transfected controls (second row). (B) FRET between the indicated combinations of CFP/YFP-tagged butyrophilin-transfected mouse 3T3 cells ±overnight challenge with HMBPP (100 ng/ml) or zoledronate (40 PM). (C) FRET between the BTN2A1 and BTN3A1 ectodomains, as measured by anti-BTN2A1 (clone 259) and anti-BTN3A1 (clone 103.2) co-staining, ±overnight challenge with HMBPP (100 ng/ml) or zoledronate (40 μM). All plots are pre-gated on transfected cells (CFP, or YFP, or both), except untransfected controls, as appropriate. Data in (A) representative of four independent experiments; (B) and (C) representative of two independent experiments.

FIG. 21. Intracellular domain association between BTN2A1 and BTN3A1 is disrupted by anti-BTN2A1 mAbs.

Percentage of FRET⁺ cells between CFP or YFP-tagged BTN2A1 and BTN3A1 (gray) following incubation of transfected mouse NIH-3T3 cells with a panel of unconjugated anti-BTN2A1 mAb (10 μg/ml), or isotype control (mouse IgG2a, κ, clone BM4). FRET levels of control BTN3A1+BTNL8 transfectants are also shown (dark gray). Data for BTN2A1+BTN3A1 group are representative of two independent experiments, each performed with BTN2A1^{CFP}+BTN3A1^{YFP} and BTN3A1^{CFP}+BTN2A1^{YFP} transfectants (pooled together on graph); BTN3A1^{CFP}+BTNL8^{YFP} are from two independent experiments.

FIG. 22. Normal γδTCR expression and responsiveness to anti-CD3 stimulation by Jurkat.G115 γδTCR mutants. (A) CD3ε/GFP co-expression on transfected HEK-293T cells with each of the Jurkat G115 γδTCR mutants. Gates depict cells used to determine BTN2A1 tetramer staining intensity. (B) Representative BTN2A1 tetramer staining (dark gray) and streptavidin alone control (gray) of each of the populations gated in (A). (C) Representative CD69 induction on Jurkat G115 mutants in co-cultures containing LM-MEL-75 WT APCs with (dark gray) or without (gray) 40 μM zoledronate. (D) CD69 induction on Jurkat G115 γδTCR mutants following overnight culture on platebound anti-CD3/anti-CD28 (10 μg/ml each, dark gray), or alone (gray). Data in (D) depict mean±SEM of n=2 independent experiment. ND, not done.

FIG. 23. Complex N-glycans are not required for BTN2A1 binding to Vγ9Vδ2⁺ γδ TCR. BTN2A1 ectodomain with complex glycans was produced in mammalian Expi293F, and BTN2A1 ectodomain with simple glycans was produced in GNTI-defective HEK-293S cells. The latter was treated with endoglycosidase H in GlycoBuffer 3 overnight at room temperature according to manufacturer instructions (NEB) to yield deglycosylated BTN2A1. (A) SDS-PAGE of the different biotinylated BTN2A1 ectodomains. (B) Phycoerythrin-conjugated tetramers produced from each batch of biotinylated BTN2A1 ectodomain, or control streptavidin (SAv.) alone were used to co-stain parental (TCR⁻) J.RT3-T3.5 (top row), J.RT3-T3.5.9C2 Vγ5Vδ1⁺ γδ TCR (middle), and Jurkat J.RT3-T3.5.G115 Vγ9Vδ2⁺ γδ TCR (bottom row) and cell lines along with anti-CD3ε-allophycocyanin. FRET between BTN2A1 tetramer and anti-CD3ε (lower histograms) was also measured in each sample. (C) Staining of glycosylated (complex or simple) BTN2A1 tetramer on a PBMC donor (left) or n=3 samples of purified and in vitro-expanded Vδ2⁺ γδ T cells (right hand plots).

FIG. 24 BTN2A1 is expressed on circulating monocytes. Anti-BTN2A1 clone 259 and clone 229 which are not cross-reactive to BTN2A2, staining of gated leukocyte subsets from two healthy PBMC donors, compared to isotype control (IgG2a, κ) or secondary alone (white) staining. Histograms depict staining on: B cells (CD19⁺ CD3⁻), CD4⁺ T cells (CD3⁺ CD4⁺ CD8⁻), CD8⁺ T cells (CD3⁺ CD4⁻ CD8⁺), γδ T cells (CD3⁺ γδTCR⁺), MAIT cells (CD3⁺ MR1-5-OP-RU tetramer⁺), NK cells (CD3⁻ CD56⁺), and monocytes (CD14⁺). Parental LM-MEL-62 and BTN2A1^{null} were included within the same experiment (lower histograms). (B) As per (A), except graphs depict mean fluorescence intensity (MFI) staining of n=4-5 donors. (C) Western immunoblot analysis of BTN2A1 and control GAPDH on in vitro-expanded Vδ2⁺ γδ T cells from five independent donors, compared to parental LM-MEL-62 and BTN2A1^{null1} cells.

FIG. 25. BTN2A1 is important for phosphoantigen-induced cytokine production by gamma delta T cells. Indicated LM-MEL-62 cells (WT or BTN2A1-KO) were co-cultured with isolated gamma-delta T cells (effector to target ratio of 2:1) and treated with zoledronate. Culture supernatants were collected after 1 day and 3 days and subjected to cytokine analysis (using Luminex kit). Data points are single wells from independently cultured and treated.

FIG. 26. Shows that anti-BTN2A1 antibodies 244, 253 and 259 exhibit stimulatory activity on human Vγ9Vδ2⁺ γδ T cells. (A) CD25 expression on in vitro-pre-expanded Vγ9Vδ2+ γδ T cells following overnight culture ±10 μg/ml anti-BTN2A1 antibody, or isotype control (IgG2a clone BM4) as indicated. (B) Interferon-γ production from the same cultures, detected by cytometric bead array. Data represent n=8 donors pooled from two separate experiments.

FIG. 27. Shows that anti-BTN2A1 antibodies 253 and 259 can induce lysis of tumor cells. (A) Tumour cell lysis by Vγ9Vδ2+ γδ T cells cultured in the presence of LM-MEL-75 (light grey bars and circle symbols) or LM-MEL-62 (dark grey bars and square symbols) and antibody 253, 259, BM4 (isotype control), zoledronate (positive control) or HMBPP (positive control). (B) CD25 expression on the same Vγ9Vδ2+ γδ T cells as in FIG. 19A.

FIG. 28 (A) Shows activation of Vγ9Vδ2 independent of phosphoantigen with anti-BTN2A1 antibodies-253 and 259 by CD25 upregulation. Antibody 259 has a higher activation potential. No addition of APCs or phosphoantigen is necessary for the antibodies to exert their activation potential. (B) Viability of LM-MEL-62 cells after co-culture with Vγ9Vδ2 cells at a 1:1 ratio and different amounts of antibodies 253 or 259 respectively. Maximum killing seems to be reached for both antibodies between 1 and 10 μg/ml with antibody 259 being a more potent inducer of cell killing than antibody 253. (C) Viability of LM-MEL-62 cells after co-culture with Vγ9Vδ2 cells at different effector to target cell (E:T) ratios, with Vγ9Vδ2 being the effectors and LM-MEL-62 cells being the targets. Vγ9Vδ2 were derived from either a melanoma patient (Patient 1) or a healthy donor and treated with antibody 259 or Zoledronate to activate Vγ9Vδ2 cells. Decrease in viability of target cells with increased effector cell numbers in both treatment groups and donors shows dependency of cell death on Vγ9Vδ2 cells.

FIG. 29 Cytokine/chemokines profile from Vγ9Vδ2 cells expanded from a melanoma patient (A and B) or from a healthy donor (C and D) Scatter plot shows mean values from 2 independent replicas. Values at 0 were set to 0.1 to allow appearance on log scale. Cytokine/chemokines not shown were not detected.

FIG. 30 (A and B). Shows percent change in expression of indicated cytokine/chemokines under the different treatments when compared to BM4 (isotype treatment). Bars show percent change of the mean value of 2 values with the exception of 259 where only one well was used. Cytokine/chemokines not shown were not detected.

FIG. 31. BTN2A1 augments activation of Vγ9Vδ1+ T cell lines to their cognate TCR ligands. (A) Representative CD69 histograms and (B) CD69 median fluorescence intensity on T cell lines expressing a Vγ9Vδ1+ TCR that reacts with human CD1c, or a Vγ9Vδ1+ TCR that reacts with human CD1d, or a Vγ5Vδ1+ TCR (9C2) that reacts with human CD1d, following a 24 h co-culture with mouse 3T3 cell APCs transfected with the indicated combinations of CD1c, CD1d, BTN2A1 or control BTNL3. Data are pooled from n=4 independent experiments.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

The description and definitions of variable regions and parts thereof, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

The terms “Butyrophilins (BTNs)” and “butyrophilin like (BTNL)” molecules refer to regulators of immune responses that belong to the immunoglobulin (Ig) superfamily of transmembrane proteins. They are structurally related to the B7 family of co-stimulatory molecules and have similar immunomodulatory functions. BTNs are implicated in T cell development, activation and inhibition, as well as in the modulation of the interactions of T cells with antigen presenting cells and epithelial cells. Certain BTNs are genetically associated with autoimmune and inflammatory diseases. The human butyrophilin family includes seven members that are subdivided into three subfamilies: BTN1, BTN2 and BTN3. The BTN1 subfamily contains only the prototypic single copy BTN1A1 gene, whereas the BTN2 and BTN3 subfamilies each contain three genes BTN2A1, BTN2A2 and BTN2A3, and BTN3A1, BTN3A2 and BTN3A3, respectively. BTNL proteins share considerable homology to the BTN family members. The human genome contains four BTNL genes: BTNL2, 3, 8 and 9.

Butyrophilins and BTNL molecules contain two Immunoglobulin-like domains: an N-terminal Ig-V-like (referred to herein as “IgV”) and a C-terminal Ig-C-like domain (referred to herein as “IgC”).

For the purposes of nomenclature only and not limitation, the amino acid sequence of a BTN2A1 is taught in NCBI RefSeq NP_001184162.1, NP_001184163.1, NP_008980.1 or NP_001184163.1 and/or in SEQ ID NOs: 1-4. In one example, the BTN2A1 is human BTN2A1.

The term “γδ T cells” refers to cells that express γ and δ chains as part of a T-cell receptor (TCR) complex. The γδ TCR is comprised of a γ-chain and δ-chain, each containing a variable and constant Ig domain. The domains are formed by genetic recombination of variable (V), diversity (D) (for TCRδ only), joining (J), and constant (C) genes within the TCRδ and γ loci. The variable domain of each chain contains 3 solvent-exposed loops that typically contact ligand, known as the CDR1, CDR2 and CDR3 regions, the latter of which is highly diverse in composition due to the V-D-J combinatorial diversity and non-template nucleotide changes (additions and deletions) at the V-D and D-J recombination sites.

In humans, the γδ T cells can be further divided into “Vδ2” and “non-Vδ2 cells,” the latter consisting of mostly Vδ1− and rarely Vδ3− or Vδ5-chain expressing cells with Vδ4, Vδ6, Vδ7, Vδ8 also described. γδ T cells can mediate antibody-dependent cell-mediated cytotoxicity (ADCC) and phagocytosis and can rapidly react toward pathogen-specific antigens without prior differentiation or expansion. γδ T-cells respond directly to proteins and non-peptide antigens and are therefore not MHC restricted. At least some γδ T-cell specific antigens display evolutionary conserved molecular patterns, found in microbial pathogens and induced self-antigens, which become upregulated by cellular stress, infections, and transformation. Such antigens are referred to herein generally as “phosphoantigens” or pAgs. Vγ9+ γδ T-cells may also respond to other antigens and ligands via TCR and (co-)receptors.

In addition, γδ T cells can be further categorized into a suite of multiple functional populations as follows: IFN-γ-producing γδ T cells, IL-17A-producing γδ T cells, antigen-presenting γδ T cells, follicular b helper γδ T cells, and regulatory γδ T cells. γδ T cells can promote immune responses exerting direct cytotoxicity, cytokine production and indirect immune responses. For example, the IFN-γ-producing phenotype is characterized by increased CD56 expression and enhanced cytolytic responses. Some γδ T cell subsets may contribute to disease progression by facilitating inflammation and/or immunosuppression. For example, IL-17A-producing γδ T cells broadly participate in inflammatory responses, having pathogenic roles during infection and autoimmune diseases.

The complementarity-determining region 3 (CDR3) regions of both γ and δ genes form quite large bulges on the top of the receptor. The human TCR composed of the Vγ9 and Vδ2 chains is characterized by an elbow angle at the C-V junction. In the CDR2 loop of Vδ, the C″ strand pairs with the C′ strand of the inner β-sheet of the domain.

The term “BTN2A1 agonist” refers to a molecule that specifically binds BTN2A1 and induces or enhances Vγ9+ γδ TCR activation. For example, the agonist binds one or more of extracellular domains (IgV and/or IgC) of the BTN2A1 molecule. The agonist BTN2A1 may induce or enhance Vγ9⁺Vδ2⁺ and/or Vγ9+VδS⁻ γδ TCR activation. For example, the agonist BTN2A1 may induce or enhance Vγ9+ γδ TCR activation, including but not limited to, Vγ9+Vδ2⁺ and/or Vγ9+Vδ1⁺ γδ TCR activation. The activation may be antigen-independent. For example, without being bound by theory or motivation, binding of the BTN2A1 agonist to BTN2A1 may modify one or more of extracellular domains (IgV and/or IgC) of the BTN2A1 molecule in such a way that mimics antigen (e.g., pAg) activation as the switch from non-stimulatory BTN2A1 to that of stimulatory. The BTN2A1 agonist may induce Vγ9+ γδ TCR activation with similar kinetics and potency as antigen binding. In one embodiment, binding of the BTN2A1 agonist leads to changes in the organization of BTN2A1 molecules on the cell surface of, for example, tumor cells, monocytes, macrophages, dendritic cells, and/or natural killer (NK) cells. For example, the BTN2A1 agonist may promote formation of a BTN2A1/BTN3 complex, for example, a BTN2A1/BTN3A1 complex, on the cell surface. The agonist may cross react with BTN3A1 or may be bi-specific for BTN2A1 and a BTN3 molecule, for example, BTN3A1. In another or further embodiment, binding of the BTN2A1 agonist induces ligation of Vγ9+ TCR on γδ T cells and/or increases the activity and/or survival of cells that express BTN2A1. A BTN2A1 agonist is stimulatory for γδ T cells and may activate one or more of cytolytic function, cytokine production of one or more cytokines, or proliferation of the γδ T cells.

The term “BTN2A1 antagonist” refers to a molecule that specifically binds BTN2A1 and inhibits Vγ9+ γδ TCR activation. For example, the antagonist binds one or more of extracellular domains (IgV and/or IgC) of the BTN2A1 molecule. The BTN2A1 antagonist may inhibit Vγ9⁺Vδ2⁺ and/or Vγ9⁺Vδ2⁻ γδ TCR activation. For example, the BTN2A1 antagonist may inhibit Vγ9⁺Vδ2⁺ and/or Vγ9⁺Vδ1⁺ γδ TCR activation. Exemplary BTN2A1 antagonists bind one or more of extracellular domains (IgV and/or IgC) of the BTN2A1 molecule and inhibit antigen (e.g., pAg) activation, binding to the Vγ9+ γδ TCR, and/or preventing the interaction with a BTN3 molecule, for example, BTN3A1. The BTN2A1 antagonist may induce a conformational change that switches the BTN2A1 molecule from stimulatory BTN2A1 to that of non-stimulatory so as to for example, prevent antigen activation and/or interaction with BTN3A1. The BTN2A1 antagonist may bind to a site on the BTN2A1 molecule that interacts with the Vγ9+ TCR or a site on the BTN2A1 molecule that interacts with a BTN3 molecule, for example BTN3A1. For example, the BTN2A1 antagonist may be a soluble TCR. In another example, the BTN2A1 antagonist may cross react with BTN3A1 or may be bi-specific for BTN2A1 and a BTN3 molecule, for example, BTN3A1. A BTN2A1 antagonist is inhibitory for γδ T cells and may inhibit one or more of cytolytic function, cytokine production of one or more cytokines, or proliferation of the γδ T cells.

As used herein, the term “inhibit(s)” or “inhibiting” in the context of γδ T cell activation shall be understood to mean that a BTN2A1 antagonist of the present disclosure reduces or decreases the level of Vγ9+ γδ TCR activation. It will be apparent from the foregoing that the BTN2A1 antagonist of the present disclosure need not completely inhibit activation, rather it need only reduce activity by a statistically significant amount, for example, by at least about 10%, or about 20%, or about 30%, or about 40%, or about 50%, or about 60%, or about 70%, or about 80%, or about 90%, or about 95%. Methods for determining inhibition of Vγ9+ γδ TCR activation are known in the art and/or described herein.

As used herein, the term “BTN2A1/BTN3 complex” refers to a complex of a BTN2A1 and a BTN3 molecule, for example, BTN2A1 and BTN3A1 complex, on the surface of a cell, for example a tumor cell, monocytes, macrophages, dendritic cells, a parenchymal cell, and/or natural killer (NK) cells. The BTN2A1/BTN3 complex may be a heteromeric complex or a multimeric complex. The complex may comprise one or more BTN3 molecules such as BTN3A1 and BTN3A2 and/or other proteins such as ATP-binding cassette transporter A1 (ABCA1). The complex may comprise BTN2A1 dimers. Similarly, the BTN3 molecule may be present in monomer or dimeric form. The BTN2A1 and BTN3 molecules may co-localize on the cell surface, or may associate either directly (e.g., cross-linked) or indirectly (via another molecule or protein).

The BTN2A1/BTN3 complex may bind antigen either directly or indirectly. For example, a cytoplasmic domain of BTN2A1 and/or a BTN3 molecule may bind antigen either directly or indirectly.

As used herein, the term “cancer” refers to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness.

As used herein, the term “binds” in reference to the interaction of a binding region of BTN2A1 agonist or antagonist with a BTN2A1 molecule means that the interaction is dependent upon the presence of a particular structure (e.g., epitope) on the BTN2A1 molecule. For example, an antibody recognizes and binds to a specific protein structure rather than to proteins generally.

If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” shall be taken to mean that the binding interaction between the binding region on the BTN2A1 agonist or antagonist and BTN2A1 molecule is dependent on the presence of the antigenic determinant or epitope. The binding region preferentially binds or recognizes a specific antigenic determinant or epitope even when present in a mixture of other molecules or organisms. In one example, the binding region reacts or associates more frequently, more rapidly, with greater duration and/or with greater affinity with the specific component or cell expressing same than it does with alternative antigens or cells. It is also understood by reading this definition that, for example, a binding region the specifically binds to a particular component may or may not specifically bind to a second antigen. As such, “specific binding” does not necessarily require exclusive binding or non-detectable binding of another antigen. The term “specifically binds” can be used interchangeably with “selectively binds” herein. Generally, reference herein to binding means specific binding, and each term shall be understood to provide explicit support for the other term. Methods for determining specific binding will be apparent to the skilled person. For example, a binding protein comprising the binding region of the disclosure is contacted with the component or a cell expressing same or a mutant form thereof or an alternative antigen. The binding to the component or mutant form or alternative antigen is then determined and a binding region that binds as set out above is considered to specifically bind to the component. In one example, “specific binding” to the component or cell expressing same, means that the binding region binds with an equilibrium constant (K_{D}) of 10 μM or less, such as 9 μM or less, 8 μM or less, 7 μM or less, 6 μM or less, 5 μM or less, 4 μM or less, 3 μM or less, 2 μM or less, or 1 μM or less such as 100 nM or less, such as 50 nM or less, for example 20 nM or less, such as, 1 nM or less, e.g., 0.8 nM or less, 1×10⁻⁸ M or less, such as 5×10⁻⁹ M or less, for example, 3×10⁻⁹ M or less, such as 2.5×10⁻⁹ M or less.

The term “recombinant” shall be understood to mean the product of artificial genetic recombination. Accordingly, in the context of an antibody or antigen binding fragment thereof, this term does not encompass an antibody naturally occurring within a subject's body that is the product of natural recombination that occurs during B cell maturation. However, if such an antibody is isolated, it is to be considered an isolated protein comprising an antibody variable region. Similarly, if nucleic acid encoding the protein is isolated and expressed using recombinant means, the resulting protein is a recombinant protein. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond.

Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The skilled artisan will be aware that an “antibody” is generally considered to be a protein that comprises a variable region made up of a plurality of polypeptide chains, e.g., a polypeptide comprising a light chain variable region (V_{L}) and a polypeptide comprising a heavy chain variable region (V_{H}). An antibody also generally comprises constant domains, some of which can be arranged into a constant region, which includes a constant fragment or fragment crystallizable (Fc), in the case of a heavy chain. A V_{H} and a V_{L} interact to form an Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies, synhumanized antibodies and chimeric antibodies. The term “antibody” also includes variants missing an encoded C-terminal lysine residue, a deamidated variant and/or a glycosylated variant and/or a variant comprising a pyroglutamate, e.g., at the N-terminus of a protein (e.g., antibody) and/or a variant lacking a N-terminal residue, e.g., a N-terminal glutamine in an antibody or V region and/or a variant comprising all or part of a secretion signal. Deamidated variants of encoded asparagine residues may result in isoaspartic, and aspartic acid isoforms being generated or even a succinamide involving an adjacent amino acid residue. Deamidated variants of encoded glutamine residues may result in glutamic acid. Compositions comprising a heterogeneous mixture of such sequences and variants are intended to be included when reference is made to a particular amino acid sequence.

In the context of the present disclosure, the term “half antibody” refers to a protein comprising a single antibody heavy chain and a single antibody light chain. The term “half antibody” also encompasses a protein comprising an antibody light chain and an antibody heavy chain, wherein the antibody heavy chain has been mutated to prevent association with another antibody heavy chain.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”. According to the numbering system of Kabat, V_{H} FRs and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FRI), 24-34 (CDR1), 35-49 (FR2), 50-56 (CDR2), 57-88 (FR3), 89-97 (CDR3) and 98-107 (FR4).

“Framework regions” (hereinafter FR) are those variable domain residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an antibody, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy (C_{H})1, a linker, a C_{H}2 and a C_{H}3. In a heavy chain, a constant region comprises a Fc. In a light chain, a constant region generally comprises one constant domain (a C_{L}i).

The term “fragment crystalizable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Furthermore, heavy chains of various subclasses (such as the IgG subclasses of heavy chains) are responsible for different effector functions and thus, by choosing the desired heavy chain constant region, proteins with desired effector function can be produced. Exemplary heavy chain constant regions are gamma 1 (IgG₁), gamma 2 (IgG₂) and gamma 3 (IgG₃), or hybrids thereof.

An “antigen binding fragment” of an antibody comprises one or more variable regions of an intact antibody. Examples of antibody fragments include Fab, Fab′, F(ab′)₂ and Fv fragments; diabodies; linear antibodies; single-chain antibody molecules, half antibodies and multispecific antibodies formed from antibody fragments.

The term “stabilized IgG₄ constant region” will be understood to mean an IgG₄ constant region that has been modified to reduce Fab arm exchange or the propensity to undergo Fab arm exchange or formation of a half-antibody or a propensity to form a half antibody. “Fab arm exchange” refers to a type of protein modification for human IgG₄, in which an IgG₄ heavy chain and attached light chain (half-molecule) is swapped for a heavy-light chain pair from another IgG₄ molecule. Thus, IgG₄ molecules may acquire two distinct Fab arms recognizing two distinct antigens (resulting in bispecific molecules). Fab arm exchange occurs naturally in vivo and can be induced in vitro by purified blood cells or reducing agents such as reduced glutathione.

As used herein, the term “monospecific” refers to a binding region comprising one or more antigen binding sites each with the same epitope specificity. Thus, a monospecific binding region can comprise a single antigen binding site (e.g., a Fv, scFv, Fab, etc) or can comprise several antigen binding sites that recognize the same epitope (e.g., are identical to one another), e.g., a diabody or an antibody. The requirement that the binding region is “monospecific” does not mean that it binds to only one antigen, since multiple antigens can have shared or highly similar epitopes that can be bound by a single antigen binding site. A monospecific binding region that binds to only one antigen is said to “exclusively bind” to that antigen.

The term “multispecific” refers to a binding region comprising two or more antigen binding sites, each of which binds to a distinct epitope, for example each of which binds to a distinct antigen. For example, the multispecific binding region may include antigen binding sites that recognize two or more different epitopes of the same protein or that may recognize two or more different epitopes of different proteins (e.g., on BTN2A1 and a BTN3 molecule such as BTN3A1).

In one example, the binding region may be “bispecific”, that is, it includes two antigen binding sites that specifically bind two distinct epitopes. For example, a bispecific binding region specifically binds or has specificities for two different epitopes on the same protein. In another example, a bispecific binding region specifically binds two distinct epitopes on two different proteins (e.g., BTN2A1 and a BTN3 molecule such as BTN3A1).

As used herein a “soluble T cell receptor” or “soluble TCR” refers to a TCR consisting of the chains of a full-length (e.g., membrane bound) receptor, except that, minimally, the transmembrane region of the receptor chains are deleted or mutated so that the receptor, when expressed by a cell, will not associate with the membrane. Most typically, a soluble receptor will consist of only the extracellular domains of the chains of the wild-type receptor (i.e., lacks the transmembrane and cytoplasmic domains). Soluble γδ TCRs of the disclosure are composed of a heterodimer of a γ chain comprising Vγ9 and a δ chain (referred to herein as “soluble V□9+ TCRs”). Various specific combinations of γ and δ chains are preferred for use in the soluble γδ TCRs of the invention, and particularly those corresponding to γδ TCR subsets that are known to exist in vivo but it is to be understood that soluble TCRs having virtually any combination of a γ chain comprising a Vγ9 and δ chains are also contemplated for use in the present disclosure.

Preferably, soluble γδ TCRs comprise γ and δ chains derived from the same animal species (e.g., murine, human).

As used herein, the terms “disease”, “disorder” or “condition” refers to a disruption of or interference with normal function, and is not to be limited to any specific condition, and will include diseases or disorders.

As used herein, a subject “at risk” of developing a disease or condition or relapse thereof or relapsing may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

As used herein, the terms “treating”, “treat” or “treatment” include administering a protein described herein to thereby reduce or eliminate at least one symptom of a specified disease or condition or to slow progression of the disease or condition.

As used herein, the term “preventing”, “prevent” or “prevention” includes providing prophylaxis with respect to occurrence or recurrence of a specified disease or condition. An individual may be predisposed to or at risk of developing the disease or disease relapse but has not yet been diagnosed with the disease or the relapse.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of a disease or condition as described herein. In some examples of the present disclosure, the term “effective amount” is meant an amount necessary to effect Vγ9+ TCR γδ T cell activation or inhibit activation of Vγ9+ TCR γδ T cells. In some examples of the present disclosure, the term “effective amount” is meant an amount necessary to effect one or more of cytolytic function, cytokine production of one or more cytokines, or proliferation of γδ T cells or inhibition of one or more of cytolytic function, cytokine production of one or more cytokines, or proliferation of γδ T cells. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number of binding proteins. The effective amount can be administered in a single dose or in a dose repeated once or several times over a treatment period.

A “therapeutically effective amount” is at least the minimum concentration required to effect a measurable improvement of a particular disease or condition. A therapeutically effective amount herein may vary according to factors such as the disease state, age, sex, and weight of the patient, and the ability of the antibody or antigen binding fragment thereof to elicit a desired response in the individual. A therapeutically effective amount is also one in which any toxic or detrimental effects of the antibody or antigen binding fragment thereof are outweighed by the therapeutically beneficial effects.

As used herein, the term “prophylactically effective amount” shall be taken to mean a sufficient quantity of a BTN2A1 agonist or antagonist to prevent or inhibit or delay the onset of one or more detectable symptoms of a disease or condition or a complication thereof.

As used herein, the term “subject” shall be taken to mean any animal including humans, for example a mammal. Exemplary subjects include but are not limited to humans and non-human primates. For example, the subject is a human.

### Antibodies

In one example, the BTN2A1 agonist or antagonist of the present disclosure the protein comprising an antigen binding domain comprises an antibody or antigen binding fragment thereof.

### Immunization-Based Methods

Methods for generating antibodies are known in the art and/or described in Harlow and Lane (editors) Antibodies: A Laboratory Manual, Cold Spring Harbor Laboratory, (1988). Generally, in such methods a protein or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (mAbs).

Monoclonal antibodies are one exemplary form of antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human immunoglobulin proteins and, for example, do not express murine immunoglobulin proteins, can also be used to generate an antibody of the present disclosure (e.g., as described in WO2002066630).

Following immunization, somatic cells with the potential for producing antibodies, e.g., B lymphocytes (B cells), are selected for use in the mAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemistry and/or immunoassay (e.g. radioimmunoassay, enzyme immunoassay, cytotoxicity assay, plaque assay, dot immunoassay, and the like).

Alternatively, ABL-MYC technology (NeoClone, Madison Wis. 53713, USA) is used to produce cell lines secreting MAbs (e.g., as described in Largaespada et al, J. Immunol. Methods. 197: 85-95, 1996).

### Library-Based Methods

The present disclosure also encompasses screening of libraries of antibodies or antigen binding fragments thereof (e.g., comprising variable regions thereof).

Examples of libraries contemplated by this disclosure include naïve libraries (from unchallenged subjects), immunized libraries (from subjects immunized with an antigen) or synthetic libraries. Nucleic acid encoding antibodies or regions thereof (e.g., variable regions) are cloned by conventional techniques (e.g., as disclosed in Sambrook and Russell, eds, Molecular Cloning: A Laboratory Manual, 3rd Ed, vols. 1-3, Cold Spring Harbor Laboratory Press, 2001) and used to encode and display proteins using a method known in the art. Other techniques for producing libraries of proteins are described in, for example in U.S. Pat. No. 6,300,064 (e.g., a HuCAL library of Morphosys AG); U.S. Pat. Nos. 5,885,793; 6,204,023; 6,291,158; or U.S. Pat. No. 6,248,516.

The antigen binding fragments according to the disclosure may be soluble secreted proteins or may be presented as a fusion protein on the surface of a cell, or particle (e.g., a phage or other virus, a ribosome or a spore). Various display library formats are known in the art. For example, the library is an in vitro display library (e.g., a ribosome display library, a covalent display library or a mRNA display library, e.g., as described in U.S. Pat. No. 7,270,969). In yet another example, the display library is a phage display library wherein proteins comprising antigen binding fragments of antibodies are expressed on phage, e.g., as described in U.S. Pat. Nos. 6,300,064; 5,885,793; 6,204,023; 6,291,158; or U.S. Pat. No. 6,248,516. Other phage display methods are known in the art and are contemplated by the present disclosure. Similarly, methods of cell display are contemplated by the disclosure, e.g., bacterial display libraries, e.g., as described in U.S. Pat. No. 5,516,637; yeast display libraries, e.g., as described in U.S. Pat. No. 6,423,538 or a mammalian display library.

Methods for screening display libraries are known in the art. In one example, a display library of the present disclosure is screened using affinity purification, e.g., as described in Scopes (In: Protein purification: principles and practice, Third Edition, Springer Verlag, 1994). Methods of affinity purification typically involve contacting proteins comprising antigen binding fragments displayed by the library with a target antigen (e.g., BTN2A1) and, following washing, eluting those domains that remain bound to the antigen.

Any variable regions or scFvs identified by screening are readily modified into a complete antibody, if desired. Exemplary methods for modifying or reformatting variable regions or scFvs into a complete antibody are described, for example, in Jones et al., J Immunol Methods. 354:85-90, 2010; or Jostock et al., J Immunol Methods, 289: 65-80, 2004; or WO2012040793. Alternatively, or additionally, standard cloning methods are used, e.g., as described in Ausubel et al (In: Current Protocols in Molecular Biology. Wiley Interscience, ISBN 047 150338, 1987), and/or (Sambrook et al (In: Molecular Cloning: Molecular Cloning: A Laboratory Manual, Cold Spring Harbor Laboratories, New York, Third Edition 2001).

Deimmunized. Chimeric. Humanized. Synhumanized. Primatized and Human Antibodies or Antigen Binding Fragments

The antibodies or antigen binding fragments of the present disclosure may be may be humanized.

The term “humanized antibody” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanized antibodies also include antibodies in which one or more residues of the human protein are modified by one or more amino acid substitutions and/or one or more FR residues of the human antibody are replaced by corresponding non-human residues. Humanized antibodies may also comprise residues which are found in neither the human antibody or in the non-human antibody. Any additional regions of the antibody (e.g., Fc region) are generally human. Humanization can be performed using a method known in the art, e.g., U.S. Pat. Nos. 5,225,539, 6,054,297, 7,566,771 or U.S. Pat. No. 5,585,089. The term “humanized antibody” also encompasses a super-humanized antibody, e.g., as described in U.S. Pat. No. 7,732,578. A similar meaning will be taken to apply to the term “humanized antigen binding fragment”.

The antibodies or antigen binding fragments thereof of the present disclosure may be human antibodies or antigen binding fragments thereof. The term “human antibody” as used herein refers to antibodies having variable and, optionally, constant antibody regions found in humans, e.g. in the human germline or somatic cells or from libraries produced using such regions.

The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human antibody will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516. A similar meaning will be taken to apply to the term “human antigen binding fragment”.

The antibodies or antigen binding fragments thereof of the present disclosure may be synhumanized antibodies or antigen binding fragments thereof. The term “synhumanized antibody” refers to an antibody prepared by a method described in WO2007019620. A synhumanized antibody includes a variable region of an antibody, wherein the variable region comprises FRs from a New World primate antibody variable region and CDRs from a non-New World primate antibody variable region.

The antibody or antigen binding fragment thereof of the present disclosure may be primatized. A “primatized antibody” comprises variable region(s) from an antibody generated following immunization of a non-human primate (e.g., a cynomolgus macaque). Optionally, the variable regions of the non-human primate antibody are linked to human constant regions to produce a primatized antibody. Exemplary methods for producing primatized antibodies are described in U.S. Pat. No. 6,113,898.

In one example an antibody or antigen binding fragment thereof of the disclosure is a chimeric antibody or fragment. The term “chimeric antibody” or “chimeric antigen binding fragment” refers to an antibody or fragment in which one or more of the variable domains is from a particular species (e.g., murine, such as mouse or rat) or belonging to a particular antibody class or subclass, while the remainder of the antibody or fragment is from another species (such as, for example, human or non-human primate) or belonging to another antibody class or subclass. In one example, a chimeric antibody comprising a V_{H} and/or a V_{L} from a non-human antibody (e.g., a murine antibody) and the remaining regions of the antibody are from a human antibody. The production of such chimeric antibodies and antigen binding fragments thereof is known in the art, and may be achieved by standard means (as described, e.g., in U.S. Pat. Nos. 6,331,415; 5,807,715; 4,816,567 and 4,816,397).

The present disclosure also contemplates a deimmunized antibody or antigen binding fragment thereof, e.g., as described in WO2000034317 and WO2004108158. De-immunized antibodies and fragments have one or more epitopes, e.g., B cell epitopes or T cell epitopes removed (i.e., mutated) to thereby reduce the likelihood that a subject will raise an immune response against the antibody or protein. For example, an antibody of the disclosure is analyzed to identify one or more B or T cell epitopes and one or more amino acid residues within the epitope is mutated to thereby reduce the immunogenicity of the antibody.

### Bispecific Antibodies

The antibodies or antigen binding fragments of the present disclosure may be bispecific antibodies or fragments thereof. A bispecific antibody is a molecule comprising two types of antibodies or antibody fragments (e.g., two half antibodies) having specificities for different antigens or epitopes. Exemplary bispecific antibodies bind to two different epitopes of the same protein. Alternatively, the bispecific antibody binds to two different epitopes on two different proteins.

Exemplary “key and hole” or “knob and hole” bispecific proteins as described in U.S. Pat. No. 5,731,168. In one example, a constant region (e.g., an IgG₄ constant region) comprises a T366W mutation (or knob) and a constant region (e.g., an IgG₄ constant region) comprises a T366S, L368A and Y407V mutation (or hole). In another example, the first constant region comprises T350V, T366L, K392L and T394W mutations (knob) and the second constant region comprises T350V, L351Y, F405A and Y407V mutations (hole).

Methods for generating bispecific antibodies are known in the art and exemplary methods are described herein.

In one example, an IgG type bispecific antibody is secreted by a hybrid hybridoma (quadroma) formed by fusing two types of hybridomas that produce IgG antibodies (Milstein C et al., Nature 1983, 305: 537-540). In another example, the antibody can be secreted by introducing into cells genes of the L chains and H chains that constitute the two IgGs of interest for co-expression (Ridgway, J B et al. Protein Engineering 1996, 9: 617-621; Merchant, A M et al. Nature Biotechnology 1998, 16: 677-681).

In one example, a bispecific antibody fragment is prepared by chemically cross-linking Fab's derived from different antibodies (Keler T et al. Cancer Research 1997, 57: 4008-4014).

In one example, a leucine zipper derived from Fos and Jun or the like is used to form a bispecific antibody fragment (Kostelny S A et al. J. of Immunology, 1992, 148: 1547-53).

In one example, a bispecific antibody fragment is prepared in a form of diabody comprising two crossover scFv fragments (Holliger P et al. Proc. of the National Academy of Sciences of the USA 1993, 90: 6444-6448).

### Antibody Fragments

### Single-Domain Antibodies

In some examples, an antigen binding fragment of an antibody of the disclosure is or comprises a single-domain antibody (which is used interchangeably with the term “domain antibody” or “dAb”). A single-domain antibody is a single polypeptide chain comprising all or a portion of the heavy chain variable domain of an antibody. For example, the single domain antibody is a nanobody.

### Diabodies, Triabodies, Tetrabodies

In some examples, an antigen binding fragment of the disclosure is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fv (scFv) Fragments

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

In one example, the linker comprises the sequence SGGGGSGGGGSGGGGS.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present disclosure encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Half-Antibodies

In some examples, the antigen binding fragment of the present disclosure is a half-antibody or a half-molecule. The skilled artisan will be aware that a half antibody refers to a protein comprising a single heavy chain and a single light chain. The term “half antibody” also encompasses a protein comprising an antibody light chain and an antibody heavy chain, wherein the antibody heavy chain has been mutated to prevent association with another antibody heavy chain. In one example, a half antibody forms when an antibody dissociates to form two molecules each containing a single heavy chain and a single light chain.

Methods for generating half antibodies are known in the art and exemplary methods are described herein.

In one example, the half antibody can be secreted by introducing into cells genes of the single heavy chain and single light chain that constitute the IgG of interest for expression. In one example, a constant region (e.g., an IgG₄ constant region) comprises a “key or hole” (or “knob or hole”) mutation to prevent heterodimer formation. In one example, a constant region (e.g., an IgG₄ constant region) comprises a T366W mutation (or knob). In another example, a constant region (e.g., an IgG₄ constant region) comprises a T366S, L368A and Y407V mutation (or hole). In another example, the constant region comprises T350V, T366L, K392L and T394W mutations (knob). In another example, the constant region comprises T350V, L351Y, F405A and Y407V mutations (hole). Exemplary constant region amino acid substitutions are numbered according to the EU numbering system.

### Other Antibodies and Antibody Fragments

The present disclosure also contemplates other antibodies and antibody fragments, such as:

(i) minibodies, e.g., as described in U.S. Pat. No. 5,837,821;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab3 (e.g., as described in EP19930302894).

### Stabilized Proteins

Antigen binding proteins of the present disclosure can comprise an IgG4 constant region or a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione.

In one example, a stabilized IgG4 constant region comprises a proline at position 241 of the hinge region according to the system of Kabat (Kabat et al., Sequences of Proteins of Immunological Interest Washington D.C. United States Department of Health and Human Services, 1987 and/or 1991). This position corresponds to position 228 of the hinge region according to the EU numbering system (Kabat et al., Sequences of Proteins of Immunological Interest Washington D.C. United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S-S) bonds in the same positions (see for example WO2010080538).

### Immunoglobulins and Immunoglobulin Fragments

An example of an antigen binding protein of the present disclosure is a protein comprising a variable region of an immunoglobulin, such as a TCR or a heavy chain immunoglobulin (e.g., an IgNAR, a camelid antibody).

### Heavy Chain Immunoglobulins

Heavy chain immunoglobulins differ structurally from many other forms of immunoglobulin (e.g., antibodies), in so far as they comprise a heavy chain, but do not comprise a light chain. Accordingly, these immunoglobulins are also referred to as “heavy chain only antibodies”. Heavy chain immunoglobulins are found in, for example, camelids and cartilaginous fish (also called IgNAR).

The variable regions present in naturally occurring heavy chain immunoglobulins are generally referred to as “V_{HH} domains” in camelid Ig and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

Heavy chain immunoglobulins do not require the presence of light chains to bind with high affinity and with high specificity to a relevant antigen. This means that single domain binding fragments can be derived from heavy chain immunoglobulins, which are easy to express and are generally stable and soluble.

A general description of heavy chain immunoglobulins from camelids and the variable regions thereof and methods for their production and/or isolation and/or use is found inter alia in the following references WO94/04678, WO97/49805 and WO 97/49805.

A general description of heavy chain immunoglobulins from cartilaginous fish and the variable regions thereof and methods for their production and/or isolation and/or use is found inter alia in WO2005118629.

### V-Like Proteins

In one example, an antigen binding protein of the present disclosure comprises a TCR. T cell receptors have two V-domains that combine into a structure similar to the Fv module of an antibody. Novotny et al., Proc Natl Acad Sci USA 88: 8646-8650, 1991 describes how the two V-domains of the T-cell receptor (termed alpha and beta) can be fused and expressed as a single chain polypeptide and, further, how to alter surface residues to reduce the hydrophobicity directly analogous to an antibody scFv. Other publications describing production of single-chain T-cell receptors or multimeric TCRs comprising two V-alpha and V-beta domains include WO1999045110 or WO2011107595.

Other non-antibody proteins comprising antigen binding domains include proteins with V-like domains, which are generally monomeric. Examples of proteins comprising such V-like domains include CTLA-4, CD28 and ICOS. Further disclosure of proteins comprising such V-like domains is included in WO1999045110.

### Adnectins

In one example, an antigen binding protein of the present disclosure comprises an adnectin. Adnectins are based on the tenth fibronectin type III (¹⁰Fn3) domain of human fibronectin in which the loop regions are altered to confer antigen binding. For example, three loops at one end of the β-sandwich of the ¹⁰Fn3 domain can be engineered to enable an Adnectin to specifically recognize an antigen. For further details see US20080139791 or WO2005056764.

### Anticalins

In a further example, an antigen binding protein of the disclosure comprises an anticalin. Anticalins are derived from lipocalins, which are a family of extracellular proteins which transport small hydrophobic molecules such as steroids, bilins, retinoids and lipids. Lipocalins have a rigid β-sheet secondary structure with a plurality of loops at the open end of the conical structure which can be engineered to bind to an antigen. Such engineered lipocalins are known as anticalins. For further description of anticalins see U.S. Pat. No. 7,250,297 or US20070224633.

### Affibodies

In a further example, an antigen binding protein of the disclosure comprises an affibody. An affibody is a scaffold derived from the Z domain (antigen binding domain) of Protein A of Staphylococcus aureus which can be engineered to bind to antigen. The Z domain consists of a three-helical bundle of approximately 58 amino acids. Libraries have been generated by randomization of surface residues. For further details see EP1641818.

### Avimers

In a further example, an antigen binding protein of the disclosure comprises an Avimer. Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see WO2002088171.

### DARPins

In a further example, an antigen binding protein of the disclosure comprises a Designed Ankyrin Repeat Protein (DARPin). DARPins are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomizing residues in the first α-helix and a β-turn of each repeat. Their binding interface can be increased by increasing the number of modules (a method of affinity maturation). For further details see US20040132028.

### Annexins

In one example, an antigen binding protein of the present disclosure comprises an annexin.

Annexin, also known as lipocortin, form a family of soluble proteins that bind to membranes exposing negatively charged phospholipids, particularly phosphatidylserine (PS), in a Ca2+-dependent manner. Annexins are formed by a four- (exceptionally eight-) fold repeat of 70 amino-acid domains that are highly conserved and by a variable amino (N)-terminal domain, which is assumed to be responsible for their functional specificities. Annexins are important in various cellular and physiological processes such as providing a membrane scaffold, which is relevant to changes in the cell's shape. Annexins have also been shown to be involved in trafficking and organization of vesicles, exocytosis, endocytosis and also calcium ion channel formation Annexin species II, V and XI are known to be located within the cellular membrane.

Annexin A5 is the most abundant membrane-bound annexin scaffold. Annexin A5 can form 2-dimensional networks when bound to the phosphatidylserine unit of the membrane. Annexin A5 is effective in stabilizing changes in cell shape during endocytosis and exocytosis, as well as other cell membrane processes.

Annexin species I (or Annexin A1) is preferentially located on the cytosolic face of the plasma membrane and binds to the phosphatidylserine unit of the membrane. Annexin A1 does not form 2-dimensional networks on the activated membrane.

In one example, the annexin species is an annexin derivative or variant thereof. Annexin derivatives or variants thereof are known in the art and exemplary derivatives or variants are disclosed herein. By way of example, annexin variants/derivatives are disclosed in WO199219279, WO2002067857, WO2007069895, WO2010140886, WO2012126157, Schutters et al., Cell Death and Differentiation 20: 49-56, 2013, or Ungethiim et al., J Biol Chem., 286(3):1903-10, 2011.

For example, an annexin derivative may be truncated, e.g., include one or more domains or fewer amino acid residues than the native protein, or may contain substituted amino acids. In one example, the annexin derivative is a truncated Annexin 1. For example, the truncated Annexin 1 does not comprise the N-terminal self-cleavage site (e.g., 41 N-terminal amino acids have been deleted). In one example, a modified annexin may have an N-terminal chelation site comprising an amino acid extension, such as X₁-Gly-X₂ where X₁ and X₂ are selected from Gly and Cys. In one example, an annexin derivative or a modified annexin binds to phosphatidylserine. In one example, an annexin derivative or a modified annexin binds to phosphatidylserine at a similar level as the wildtype annexin. For example, an annexin derivative or modified annexin binds to phosphatidylserine at the same level as the wildtype annexin.

In one example, an antigen binding protein of the present disclosure comprises Annexin A5. For the purposes of nomenclature only and not limitation, the amino acid sequence of an Annexin A5 is taught in Gene Accession ID 308, NCBI reference sequence NP_001145 and/or in SEQ ID NO: 5. For the purposes of nomenclature only and not limitation, the amino acid sequence of an Annexin A1 is taught in NCBI reference sequence NP_000691.1 and/or in SEQ ID NO: 7.

### Gamma-Carboxyglutamic Acid-Rich (GLA) Domains

In one example, the antigen binding protein of the present disclosure comprises a gamma-carboxyglutamic acid-rich (GLA) domain or variant thereof.

The GLA domain contains glutamate residues that have been post-translationally modified by vitamin K-dependent carboxylation to form gamma-carboxyglutamate (Gla).

Proteins known to comprise a GLA domain are known in the art and include, but are not limited to, vitamin K-dependent proteins S and Z, prothrombin, transthyretin, osteocalcin, matrix GLA protein, inter-alpha-trypsin inhibitor heavy chain H2 and growth arrest-specific protein 6.

### Lactadherin Domains

In one example, antigen binding protein of the present disclosure comprises a lactadherin domain.

Lactadherin is a glycoprotein secreted by a variety of cell types and contains two EGF domains and two C domains (C1C2 and C2) with sequence homology to the C1 and C2 domains of blood coagulation factors V and VIII. Similar to these coagulation factors, lactadherin binds to phosphatidylserine (PS)-containing membranes with high affinity.

In one example, the lactadherin domain is a C1C2 domain (e.g., as set forth in SEQ ID NO: 27). In another example, the lactadherin domain is a C2 domain.

### Protein Kinase Domains

In one example, the present disclosure provides an antigen binding protein comprising a protein kinase C domain.

Protein kinase C (PKC) is a family of protein kinase enzymes that are involved in controlling the function of other proteins through the phosphorylation of hydroxyl groups of serine and threonine amino acid residues on these proteins, or a member of this family.

The structure of PKC is known in the art and consists of a regulatory domain and a catalytic domain tethered together by a hinge region. The regulatory domain comprises a C1 and a C2 domain which bind to DAG and Ca²⁺ respectively to recruit PKC to the plasma membrane.

In one example, the protein kinase C domain is the C1 domain. In another example, the protein kinase C domain is the C2 domain.

### Pleckstrin Homology Domain

In one example, the present disclosure provides an antigen binding protein comprising a pleckstrin homology (PH) domain.

The PH domain is known in the art and is a small modular domain that occurs in a wide range of proteins involved in intracellular signaling or as a constituent of the cytoskeleton. The PH domain comprises approximately 120 amino acids. The domains can bind phosphatidylinositol within biological membranes and proteins such as the beta/gamma subunits of heterotrimeric G proteins. Through these interactions, PH domains play a role in recruiting proteins to different membranes, thus targeting them to appropriate cellular compartments or enabling them to interact with other components of the signal transduction pathways.

### Phosphatidylserine-Interacting Peptides

In one example, the present disclosure provides an antigen binding protein comprising a phosphatidylserine-interacting peptide. Suitable peptides are known in the art and include, for example, PSP1 as described in Thapa et al., J. Cell Mol. Med. 12. 1649-1660, 2008 and Kim et al., PLOS One, 10(3): e0121171. PSP1 comprises the sequence CLSYYPSYC (SEQ ID NO: 28). The present disclosure also contemplates variants of PSP1 that retain its ability to bind phosphatidylserine.

### Soluble T Cell Receptors

In one example, the BTN2A1 antagonist of the present disclosure is a soluble Vγ9+ TCR.

A soluble Vγ9+ TCR useful in the disclosure typically is a heterodimer comprising a γ chain comprising Vγ9+ γ chain and a δ chain, but multimers (e.g., tetramers) comprising two different γδ heterodimers or two of the same γδ heterodimers are also contemplated for use in the present disclosure.

Soluble Vγ9+ TCRs of the present disclosure can be produced by any suitable method known to those of skill in the art, and are most typically produced recombinantly. According to the present disclosure, a recombinant nucleic acid molecule useful for producing a soluble γδ TCR typically comprises a recombinant vector and a nucleic acid sequence encoding one or more segments (e.g., chains) of a γδ TCR. According to the present disclosure, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is, nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid sequences which encode a protein of interest (e.g., the TCR chains) or which are useful for expression of the nucleic acid molecules. The vector can be either RNA or DNA, either prokaryotic or eukaryotic, and typically is a plasmid.

Typically, a recombinant nucleic acid molecule includes at least one nucleic acid molecule of the present invention operatively linked to one or more transcription control sequences. As used herein, the phrase “recombinant molecule” or “recombinant nucleic acid molecule” primarily refers to a nucleic acid molecule or nucleic acid sequence operatively linked to a transcription control sequence, but can be used interchangeably with the phrase “nucleic acid molecule”, when such nucleic acid molecule is a recombinant molecule as discussed herein. According to the present disclosure, the phrase “operatively linked” refers to linking a nucleic acid molecule to a transcription control sequence in a manner such that the molecule is able to be expressed when transfected (i.e., transformed, transduced, transfected, conjugated or conduced) into a host cell. Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell or organism into which the recombinant nucleic acid molecule is to be introduced.

One or more recombinant molecules of the present invention can be used to produce an encoded product (e.g., a soluble γδ TCR) of the present disclosure. In one embodiment, an encoded product is produced by expressing a nucleic acid molecule as described herein under conditions effective to produce the protein. A preferred method to produce an encoded protein is by transfecting a host cell with one or more recombinant molecules to form a recombinant cell. Suitable host cells to transfect include, but are not limited to, any bacterial, fungal (e.g., yeast), insect, plant or animal cells that can be transfected. Host cells can be either untransfected cells or cells that are already transfected with at least one other recombinant nucleic acid molecule. Resultant proteins of the present invention may either remain within the recombinant cell; be secreted into the culture medium; be secreted into a space between two cellular membranes; or be retained on the outer surface of a cell membrane. The phrase “recovering the protein” refers to collecting the whole culture medium containing the protein and need not imply additional steps of separation or purification. Proteins produced according to the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Proteins produced according to the present disclosure are preferably retrieved in “substantially pure” form. As used herein, “substantially pure” refers to a purity that allows for the effective use of the soluble γδ TCR in a composition and method of the present disclosure.

By way of example, recombinant constructs containing the relevant γ and δ genes (e.g., nucleic acid sequences encoding the desired portions of the γ and δ chains of γδ TCR) can be synthesized de novo or can be produced by PCR of TCR cDNAs derived from a source of γδ T cells (e.g., hybridomas, clones, transgenic cells) that express the desired receptor. The PCR amplification of the desired γ and δ genes can be designed so that the transmembrane and cytoplasmic domains of the chains will be omitted (i.e., creating a soluble receptor). Preferably, portions of the genes that form the interchain disulfide bond are retained, so that the γδ heterodimer formation is preserved. In addition, if desired, sequence encoding a selectable marker for purification or labeling of the product or the constructs can be added to the constructs. Amplified γ and δ cDNA pairs are then cloned, sequence-verified, and transferred into a suitable vector, such as a baculoviral vector containing dual baculovirus promoters (e.g., pAcUW51, Pharmingen Corp., San Diego, Calif.).

The soluble γδ TCR DNA constructs are then co-transfected into a suitable host cell (e.g., in the case of a baculoviral vector, into suitable insect host cells or in the case of a mammalian expression vector, into suitable mammalian host cells) which will express and secrete the recombinant receptors into the supernatant, for example. Culture supernatants containing soluble γδ TCRs can then be purified using various affinity columns, such as nickel nitrilotriacetic acid affinity columns. The products can be concentrated and stored. It will be clear to those of skill in the art that other methods and protocols can be used to produce soluble TCRs for use in the present disclosure, and such methods are expressly contemplated for use herein.

### Pharmaceutical Compositions

Suitably, in compositions or methods for administration of the BTN2A1 agonist or antagonist to a subject, the BTN2A1 agonist or antagonist is combined with a pharmaceutically acceptable carrier as is understood in the art. Accordingly, one example of the present disclosure provides a composition (e.g., a pharmaceutical composition) comprising the BTN2A1 agonist or antagonist of the disclosure combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

A BTN2A1 agonist or antagonist invention is useful for parenteral, topical, oral, or local administration, aerosol administration, or transdermal administration, for prophylactic or for therapeutic treatment. In one example, the BTN2A1 agonist or antagonist is administered parenterally, such as subcutaneously or intravenously. For example, the BTN2A1 agonist or antagonist is administered intravenously.

Formulation of a BTN2A1 agonist or antagonist to be administered will vary according to the route of administration and formulation (e.g., solution, emulsion, capsule) selected. An appropriate pharmaceutical composition comprising a BTN2A1 agonist or antagonist to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The BTN2A1 agonist or antagonist can be stored in the liquid stage or can be lyophilized for storage and reconstituted in a suitable carrier prior to use according to art-known lyophilization and reconstitution techniques.

Functional Measures of γδ T cell Immune Responses

The present disclosure also relates to BTN2A1 agonists or antagonists, which can activate or inhibit the cytolytic function, cytokine production of one or more cytokines and/or proliferation of γδ T cells. T-cell number and function may be monitored by assays that detect T cells by an activity such as cytokine production, proliferation, or cytotoxicity. Such activity may be correlated with clinical outcome. For example, activation of cytolytic activity may result in lysis of tumor targets or infected cells following treatment with a BTN2A1 agonist or antagonist. Activation and increased cytokine production may lead to cytokine-induced cell death of tumor or other targets.

By activating the cytolytic function of γδ T cells, it is meant an increase of the cytotoxicity of γδ T cells, i.e., an increase of the specific lysis of the target cells by γδ T cells. By inhibiting the cytolytic function of γδ T cells, it is meant a decrease of the cytotoxicity of γδ T cells, i.e., a decrease of the specific lysis of the target cells by γδ T cells. The cytolytic function of γδ T cells can be measured by, for example, direct cytotoxicity assays. A cytotoxicity assay typically involves mixing a sample containing T cells or PBMCs with targets loaded with ⁵¹Cr or europium and measuring the release of the chromium or europium after target cell lysis. Surrogate targets are often used, such as tumor cell lines. The targets can be loaded with an antigen, for example, a pAg. The sample and targets are incubated in the presence or absence of the BTN2A1 agonist or antagonist. The percentage of lysis of the targets after incubation for approximately 4 hours is calculated by comparison with the maximum achievable lysis of the target. Cytotoxicity assays can be used for monitoring the activity of passively delivered T cells and active immunotherapy approaches.

By activating or inhibiting the cytokine production of one or more cytokines by γδ T cells, it is meant an increase or decrease, respectively, in total cytokine production of one or more particular cytokines (for example, IFN-γ, TNF-α, GM-CSF, IL-2, IL-6, IL-8, IP-10, MCP-1, MIP-1α, MIP-1β or IL-17A) by γδ T cells. Cytokine secretion by T cells may be detected by measuring either bulk cytokine production (by an ELISA), by bead based assays (e.g., Luminex), or enumerating individual cytokine producing T cells (by an ELISPOT assay).

In an ELISA assay, PBMC samples are incubated with or without added cells that express BTN2A1 in the presence or absence of a BTN2A1 agonist or antagonist, and after a defined period of time, the supernatant from the culture is harvested and added to microtiter plates coated with antibody for cytokines of interest. Antibodies linked to a detectable label or reporter molecule are added, and the plates washed and read. Typically, a single cytokine is measured in each well, although up to 15 cytokines can be measured in a single sample. Antibodies to cytokines of interest may be covalently bound to microspheres with uniform, distinctive proportions of fluorescent dyes. Detection antibodies conjugated to a fluorescent reporter dye are then added, and flow cytometry performed. By gating on a particular fluorescence indicating a particular cytokine of interest, it is possible to quantify the amount of cytokine that is proportional to the amount of reporter fluorescence.

In a bead based assay like Luminex, the sample is usually added to a mixture of color-coded beads, pre-coated with analyte-specific capture antibodies. The antibodies bind to the analytes of interest. Biotinylated detection antibodies specific to the analytes of interest are added and form an antibody-antigen sandwich. Fluorophore-conjugated streptavidin is added and binds to the biotinylated detection antibodies. Beads are read on a flow-based detection instrument. One laser classifies the bead and determines the analyte that is being detected. The second laser determines the magnitude of the fluorophore-derived signal, which is in direct proportion to the amount of analyte bound.

An ELISPOT assay typically involves coating a 96-well microtiter plate with purified cytokine-specific antibody; blocking the plate to prevent nonspecific absorption of random proteins; incubating the cytokine-secreting T cells with stimulator cells in the presence or absence of a BTN2A1 agonist or antagonist at several different dilutions; lysing the cells with detergent; adding a labeled second antibody; and detecting the antibody-cytokine complex. The product of the final step is usually an enzyme/substrate reaction producing a colored product that can be quantitated microscopically, visually, or electronically. Each spot represents one single cell secreting the cytokine of interest.

Cytokine production of one or more cytokines by γδ T cells can also be detected by multiparameter flow cytometry. Here, cytokine secretion is blocked for 4-24 hours with Brefeldin A or Monensin (both protein transport inhibitors that act on the Golgi in different ways, which one is best depends on the cytokine to examine) in γδ T cells before the cells are surface stained for markers of interest and then fixed and permeabilized followed by intracellular staining with fluorophore-coupled antibodies targeting the cytokines of interest. Afterwards the cells can be analyzed by Flow-cytometry. It is possible to monitor immune responses in humans by characterizing the cytokine secretion pattern of T cells in peripheral blood, lymph nodes, or tissues by flow cytometry. This can be done ex-vivo without BFA or Monensin treatment.

By activating or inhibiting proliferation of γδ T cells, it is meant an increase or decrease, respectively, in number of γδ T cells. Proliferation can be measured using a lymphoproliferative assay. A sample of purified T cells or PBMCs is mixed with various dilutions of stimulator cells in the presence or absence of a BTN2A1 agonist or antagonist. After 72-120 h, [³H]thymidine is added, and DNA synthesis (as a measure of proliferation) can be quantified by using a gamma counter to measure the amount of radiolabeled thymidine incorporated into the DNA. The proliferation assay can be used to compare γδ T-cell responses before and after administration of the BTN2A1 agonist or antagonist.

The present disclosure also relates to BTN2A1 agonists or antagonists, which can activate or inhibit the activity and/or survival of cells that express BTN2A1, for example, monocytes, macrophages, and/or dendritic cells. By activating or inhibiting the activity of cells that express BTN2A1, it is meant that the BTN2A1 agonists or antagonists increase or decrease, respectively, costimulatory molecule expression (like CD86, CD80 and HLA-DR) on the surface of the cells, and/or increases the proinflammatory responses induced by Toll-like receptor (TLR) ligands in these cells and/or modulating the expression of immune checkpoint molecules (like PD-L1, PD-L2). The activity and/or survival of cells that express BTN2A1 may be measured by antigen presentation assays. Briefly, CD14+ cells can be isolated from PBMCs and cultured over 5 days in media containing GM-CSF and IL-4 to produce MODCs. Antibody-protein complexes can be added to these and presentation capacity measured by adding HLA-matched T cells that can recognize the protein added to the MODCs followed by an ICS on these T cells as described previously.

### Indications

The present disclosure relates to BTN2A1 agonists or antagonists, which can be used to prevent, treat, delay the progression of, prevent a relapse of, or alleviate a symptom of a disease or condition. BTN2A1 agonists or antagonists can be administered directly to a subject in need thereof or can be used for ex vivo stimulation and adoptive transfer of cells (comprising γδ T-cells) to the subject.

The skilled person will appreciate that use of a BTN2A1 agonist or antagonist is dependent on whether you want to enhance or suppress one or more γδ T-cell immune responses and whether the γδ T-cell population targeted is immune suppressive or immune stimulatory. In one embodiment, γδ T-cell function is manipulated to promote, for example, anti-tumor or anti-pathogen activity of γδ T-cells, for example, by promoting cytotoxicity toward tumor or infected cells. In another embodiment, γδ T-cell function is manipulated to promote immunosuppressive and/or regulatory activities of γδ T-cells during immune responses.

BTN2A1 agonists or antagonists can be used to prevent, treat, delay the progression of, prevent a relapse of, or alleviate a symptom of cancer.

BTN2A1 agonists or antagonists can also be used to prevent, treat, delay the progression of, prevent a relapse of, or alleviate a symptom of infection.

BTN2A1 agonists or antagonists may be used as a vaccine adjuvant for the treatment of a cancer or an infection.

BTN2A1 agonists or antagonists can also be used to prevent, treat, delay the progression of, prevent a relapse of, or alleviate a symptom of an autoimmune disease.

BTN2A1 antagonists may be used in combination with other immunosuppressive and chemotherapeutic agents such as, but not limited to, prednisone, azathioprine, cyclosporin, methotrexate, and cyclophosphamide.

### Example 1: Materials and Methods

### Human Samples

Healthy donor blood derived human peripheral blood cells (PBMCs) were obtained from the Australian Red Cross Blood Service under ethics approval 17-08VIC-16 or 16-12VIC-03, with ethics approval from University of Melbourne Human Ethics Sub-Committee (1035100) or Olivia Newton John Cancer Research Institute (ONJCRI) Austin Health Human Research Ethics Committee (H2012-04446) and isolated via density gradient centrifugation (Ficoll-Paque PLUS GE Health care) and red blood cell lysis (ACK buffer, produced in-house). Established cell lines were routinely verified as Mycoplasma-negative using the MycoAlert test (Lonza) and cross-contamination excluded by STR profiling.

### Flow Cytometry

Human cells were pelleted (400× g), washed, and incubated at 4° C. with PBS/2% fetal bovine serum (FBS) containing human Fc receptor block (Miltenyi Biotec). Mouse NIH-3T3 cells were incubated with anti-CD16/CD32 (clone 2.4G2, produced in-house). Cells were then incubated with 7-aminoactinomycin D (7-AAD, Sigma) or LIVE/DEAD® viability markers (ThermoFisher) plus antibodies (Table 1). BTN2A1 and BTN3A were detected using monoclonal antibodies generated in-house (see below). Anti-BTN2A1 mAb or matched isotype control (clone BM4, produced in house) were conjugated to Alexa Fluor®-647 via amine coupling (Thermo Fisher), and anti-BTN3A (clone 103.2) was conjugated to R-phycoerythrin (Prozyme) using sulfo-SMCC heterobifunctional crosslinker. In some experiments, unconjugated anti-BTN2A1 mAb were detected using goat anti-mouse polyclonal secondary antibody PE (BD-Pharmingen), with a subsequent blocking step (5% normal mouse serum). Cells were also stained with tetrameric Vγ9Vδ2⁺ γδTCR, BTN2A1 or mouse CD1d-α-GalCer ectodomains (produced in house, see below), or equivalent amounts of streptavidin conjugate alone (BD). Each reagent was titrated to determine the optimal dilution factor. All data were acquired on an LSRFortessa™ II (BD), and analysed with FACSDiva and FlowJo (BD) software. All samples were gated to exclude unstable events, doublets and dead cells using time, forward scatter area versus height, and viability dye parameters, respectively.

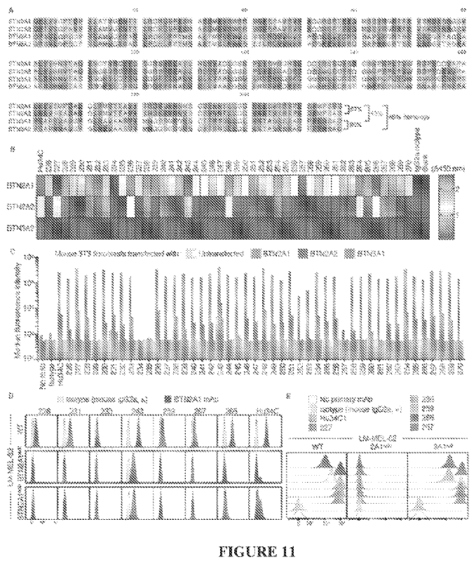

TABLE 1
Antibodies used for flow cytometry.

| | Target | Source | Clone | | | |
|---|---|---|---|---|---|---|
| Target | species | species | name | Fluorochrome | Manufacturer | Concentration |
| Fc receptor block | Human | Unknown | N.A. | None | Miltenyi | 1:40 |
| | | | | | Biotec | |
| Fc receptor block | Mouse | Rat | 2.4G2 | None | In-house | 1:50 |
| 7-AAD | Mouse/human | N.A. | N.A. | Not applicable | Sigma | 3 μg/ml |
| LIVE/DEAD | Mouse/human | N.A. | N.A. | Near-IR, Violet | ThermoFisher | 1:1,000 |
| marker | | | | | | |
| CD3ε | Human | Mouse | UCHT1 | APC | BD-Pharmingen | 1:50 |
| CD3ε | Human | Mouse | UCHT1 | BUV395 | BD-Pharmingen | 1:100 |
| CD3 | Human | Mouse | SK7 | APC-Cy7 | BioLegend | 1:100 |
| CD3ε | Human | Mouse | OKT3 | — | In-house | 1:100 |
| γδTCR | Human | Mouse | 11F2 | PE-Cy7 | BD-Pharmingen | 1:50 |
| CD19 | Human | Mouse | SJ25C1 | APC-Cy7 | BD-Pharmingen | 1:100 |
| CD4 | Human | Mouse | RPA-T4 | FITC | BD-Pharmingen | 1:20 |
| CD8α | Human | Mouse | SK1 | APC, PE | BD-Pharmingen | 1:100-200 |
| CD56 | Human | Mouse | HCD56 | BV605 | BioLegend | 1:100 |
| TCR Vδ1 | Human | Mouse | TS8.2 | FITC | Invitrogen | 1:200 |
| TCR Vδ2 | Human | Mouse | B6 | BV711 | BioLegend | 1:150-400 |
| TCR Vγ9 | Human | Mouse | B3 | APC | BioLegend | 1:400 |
| CD14 | Human | Mouse | M5E2 | BUV805 | BD-Pharmingen | 1:400 |
| CD45 | Human | Mouse | HI30 | AF700 | BioLegend | 1:150 |
| CD25 | Human | Mouse | M-A251 | PE | BD-Pharmingen | 1:50 |
| CD69 | Human | Mouse | FN50 | PE-Cy7 | BD-Pharmingen | 1:100 |
| CD69 | Human | Mouse | FN50 | PE | BD-Pharmingen | 1:50 |
| IFN-γ | Human | Mouse | 4S.B3 | PerCP-Cy5.5 | Biolegend | 1:100 |
| Isotype control | N.A. | Mouse | MOPC-21 | Unconjugated, | BioLegend | 10 μg/ml |
| IgG1, κ | | | | PE | | |
| Isotype control | N.A. | Human-m. | BM4 | Unconjugated, | In house | 2 μg/ml |
| IgG2a, κ | | IgG2a | | AF647 | | |
| BTN2A1 | Human | Human m. | See FIG. 11 | Unconjugated, | In house | 2 μg/ml |
| | | IgG2a | | AF647 | | |
| BTN3A1/3A2/3A3 | Human | Mouse | 20.1 | Unconjugated, | In house | 2 μg/ml |
| | | | | PE | | |
| BTN3A1/3A2/3A3 | Human | Mouse | 103.2 | Unconjugated, | In house | 0.3 μg/ml |
| | | | | PE | | |
| pan-Immunoglobulin | Mouse | Goat | polyclonal | BV421, PE | BioLegend | 1:40 |
| MR1-5-OP-RU | | | | | | |
| tetramer | Human | | | BV421 | In-house | 2 μg/ml |
| TCR tetramers | Human | | | PE | In-house | 5 μg/ml |
| BTN2A1 tetramer | Human | | | PE | In-house | 5 μg/ml |
| mouse CD1d | | | | | | |
| tetramer | Mouse | | | PE | In-house | 5 μg/ml |

### γδ T Cell Isolation and Expansion

In some experiments γδ T cells were enriched by MACS using either anti-γδTCR-PECy7 followed by anti-phycoerythrin-mediated magnetic bead purification, or using a γδ T cell isolation kit (Miltenyi Biotec). After enrichment CD3⁺ Vδ2⁺ γδ T cells were further purified by sorting using an Aria III (BD). Enriched γδ T cells were stimulated in vitro for 48 h with plate-bound anti-CD3ε (OKT3, 10 μg/ml, Bio-X-Cell), soluble anti-CD28 (CD28.2, 1 μg/ml, BD Pharmingen), phytohemagglutinin (0.5 μg/ml, Sigma) and recombinant human IL-2 (100 U/ml, PeproTech), followed by maintenance with IL-2 for 14-21 d. Cells were cultured in complete medium consisting of a 50:50 (v/v) mixture of RPMI-1640 and AIM-V (Invitrogen) supplemented with 10% (v/v) FCS (JRH Biosciences), penicillin (100 U/ml), streptomycin (100 μg/ml), Glutamax (2 mM), sodium pyruvate (1 mM), nonessential amino acids (0.1 mM) and HEPES buffer (15 mM), pH 7.2-7.5 (all from Invitrogen Life Technologies), plus 50 μM 2-mercaptoethanol (Sigma-Aldrich).

### Transfections

BTN2A1, BTN2A2, BTN3A1, BTN3A2, BTNL3 and BTNL8 (all isoform 1) were cloned into pMIG II mammalian expression vector (a gift from D. Vignali (Addgene plasmid #52107) (J. Holst et al. (2006)) and verified by Sanger sequencing. Mouse NIH-3T3, hamster CHO-K1, human LM-MEL-62 cells were plated out the day before and transfected using FuGene HD® or Viafect™ in OptiMEM according to manufacturer's instructions. After 48 h (72 h with LM-MEL-62 cells) to enable gene expression, cells were tested for GFP and gene expression and subsequently used in phenotyping or functional assays.

### γδ T Cell Functional Assays

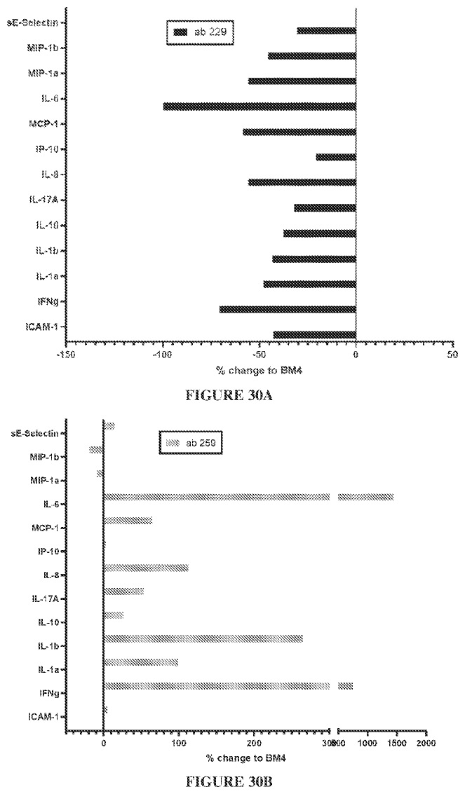

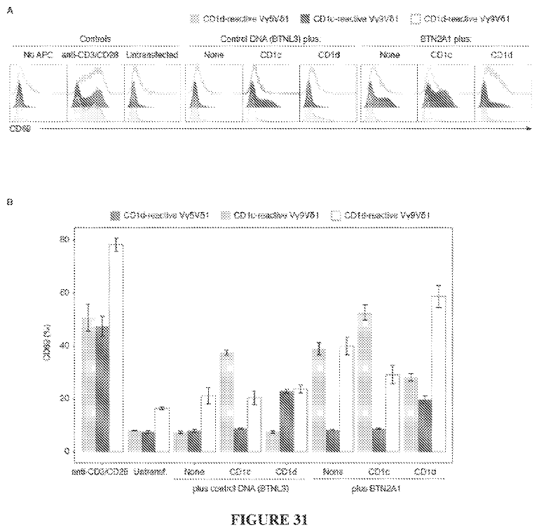

Fresh PBMC (2×10⁶) were cultured in 24 well plates ±zoledronate (4 μM, Sigma) and purified mAb against BTN2A1, BTN3A1, or isotype control IgG1κ (MOPC-21, BioLegend) (10 μg/ml) After 24 h CD3ε⁺ γδTCR⁺ Vδ2^{+/-} γδ T cell activation was assessed by flow cytometry and cytokine production was determined by cytometric bead array according to manufacturer instructions (BD). For the assays in FIG. 14, PBMC were cultured in 24 well plates and blocked for 30 min with mAb against BTN2A1, BTN3A1, or isotype control (10 μg/ml). Cells were then stimulated for 18 h with combinations of HMBPP (0.5 ng/ml, Sigma), zoledronate (4 μM, Sigma) and CEF (1 μg/ml, Miltenyi) in addition to IL-2 (25 U/ml, Miltenyi) and Golgiplug protein transport inhibitor (BD Biosciences). Cells were surface-stained and then fixed and permeabilized using Foxp3/Transcription Factor Staining Buffer Set (Invitrogen) according to the manufacturer's protocol followed by staining with anti-IFN-γ (Biolegend). For co-culture assays, purified and in vitro-expanded γδ T cells (5×10⁵) were incubated in 96 well plates with APCs (3×10⁵) for 24 h ±zoledronate (4 μM), and γδ T cell activation was determined by flow cytometry as above.

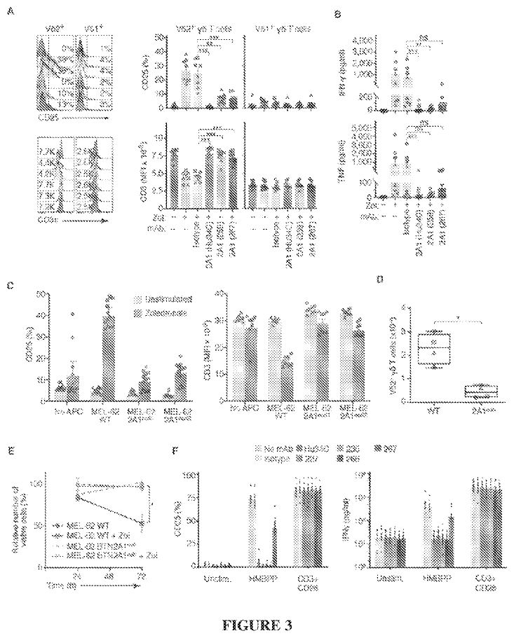

Alternatively (in FIG. 3C), 4×10⁴ primary γδ T cells purified from PBMC donors using a γδ T cell magnetic bead isolation kit (Miltenyi) were cultured at a 2:1 ratio with either LM-MEL-62 WT or BTN2A1^{null1} APC in the presence of 1 uM zoledronate for 2 days. Non-adherent cells were subsequently washed and cultured in fresh plates without APC for an additional 7 days in media plus 100 U/ml IL-2. Vδ2⁺γδ T cells were then enumerated by flow cytometry.

### FRET Assays

For detection of FRET between BTN2A1 and BTN3A1 ectodomains, cells were stained with PE-conjugated anti-BTN3A1 (donor), and Alexa 647-conjugated BTN2A1 (acceptor). FRET was detected in a compensated yellow 670/30 channel. CFP (mTurquoise2, donor) and YFP (mVenus, acceptor) constructs containing either a long (used for BTN3A1 and BTNL3) or short (used for BTN2A1 and BTNL8) flexible N-terminal linker (FIG. 19B) were synthesized (ThermoFisher) and cloned into the C-terminus of butyrophilin constructs between an in-frame MfeI site that was introduced by site-directed mutagenesis, and a 3′ SalI site, which also removed the pMIG IRES-GFP motif. CFP was detected in a violet 450/50 channel, YFP using yellow 585/15, and FRET using a violet 530/30 channel from which CFP and YFP spillover had been removed by compensation. The frequency of cells identified as FRET⁺ was examined on gated CFP⁺YFP⁺NIH-3T3 cells for dual tranfectants, and either CFP⁺ or YFP⁺ for single transfectants.

Tumor viability assays Tumor (10⁴) cells were plated out in 96 well plates in RF-10. The next day 2×10⁴ γδ T cells were added with 100 U/ml IL-2 (Miltenyi) ±1 μM zoledronate (Sigma). After a 1- or 3-day incubation, viability was assessed by an MTS assay, with absorbance measured at 490 nm on a SpectroStar Nano plate reader (BMG Labtech) and corrected for background and normalized against wells containing APCs alone at each time point.

Single cell γδTCR sequencing

CD3ε⁺ γδTCR⁺ Vδ2⁺ γδ T cells derived from healthy PBMC donors were individually sorted. The γδTCR was then amplified with primers listed in Supplementary Table 2. PCR amplicons were then cloned into pHL-sec containing either γ- or δ-chain ectodomains (FIG. 8C) for expression.

### Whole Genome CRISPR/Cas9 Knockout Screen

The CRISPR/Cas9 knockout screen was performed essentially as described J. Young et al. (2017)). Briefly, a pooled lentiviral human gRNA knockout library containing n=6 gRNA per gene (GeCKOv2, a gift from Feng Zhang, Addgene #1000000048) was transformed into Endura™ ElectroCompetent cells (Lucigen) at >500× coverage, and grown in 1 L liquid Luria Broth cultures for 16 hours at 37° C. Plasmid DNA was purified (PureLink™ gigaprep, ThermoFisher) and gRNA abundance in pre- and post-amplified libraries was validated by sequencing of PCR-amplified libraries (Illumina HiSeq, 60×10⁶ reads per sample), with <0.2% gRNA dropout. Lentiviral particles were produced by transient transfection of HEK-293T cells with the gRNA library DNA plus packaging plasmids using FuGENE® (Promega), and culture supernatant was titrated on LM-MEL-62 cells to determine the viral titre using puromycin (1 μg/ml, ThermoFisher). Four biological replicates of LM-MEL-62 cells (2×10⁸ each) were transduced with the lentiviral library at a multiplicity of infection of ˜0.3. Transduced cells were then selected with puromycin for an additional 5 d, after which Vγ9Vδ2⁺ γδTCR tetramer #6^{low} cells were sorted from half of each replicate (˜6×10⁷), and the remaining half was used as the unsorted control. The sorted cells were re-expanded for ˜2 weeks and subsequently re-sorted. This was repeated an additional 2 times in order to adequately enrich for a clear Vγ9Vδ2⁺ γδTCR tetramer #6^{low} population of LM-MEL-62 cells (FIG. 9A). Genomic DNA was then extracted as previously described S. Chen et al. (2015)), including an additional phenol-chloroform purification step. gRNA from ˜6×10⁷ unsorted and ˜3×10⁷ sorted cells was amplified from genomic DNA using PCR (33 cycles) with Pfu-based DNA polymerase (Herculase II Fusion, Agilent Technologies) and one-step primers containing index and adaptor sequences (IDT Ultramer oligos) as previously described (J. Young et al (2017)). Amplicons were gel-extracted following electrophoresis (Wizard® SV Gel Clean-Up System, Promega), quantified with PicoGreen® (ThermoFisher) and sequenced using a NovaSeq (Illumina). Sample data were demultiplexed using a combination of the forward primer stagger motifs and the reverse 8-mer barcodes using Cutadapt (M. Martin et al (2011)) and analysed using the EdgeR software package in R studio (M. D. Robinson et al. (2010)). Guides were enumerated using the processAmplicons function, allowing for a single base pair mismatch or shifted guide position. Guides with less than 0.5 counts/10⁶ in at least five samples were excluded from the analysis. After dispersion estimation, differential gRNA expression between unsorted and sorted samples was determined using the exactTest function, where a false discovery rate (FDR) of <0.05 was considered statistically significant.

### Production of Soluble Proteins

Soluble human γδTCRs, butyrophilin 2A1 and mouse CD1d ectodomains were expressed by transient transfection of mammalian Expi293F or GNTI-defective HEK-293S cells using ExpiFectamine or PEI, respectively, with pHL-sec vector DNA encoding constructs with C-terminal biotin ligase (AviTag™) and His₆ tags (A. R. Aricescu et al. (2006)). MR1-5-OP-RU tetramer was produced as previously described (H. F. Koay et al. (2019)). Protein was purified from culture supernatant using immobilized metal affinity chromatography (IMAC) and gel filtration, and enzymatically biotinylated using BirA (produced in-house). Proteins were re-purified by size exclusion chromatography and stored at −80° C. Biotinylated proteins were tetramerized with streptavidin-PE (BD) at a 4:1 molar ratio. DNA constructs encoding butyrophilin B30.2 intracellular domains with C-terminal His₆ tags were synthesized de novo (ThermoFisher) and cloned into pET-30 bacterial expression vectors. BL21 DE3 (pLysS) E. coli were used for overnight expressions at 30° C. following induction with IPTG (1 mM). Cell pellets were washed and lysed using a sonicator in PBS/1 mM DTT and B30.2 proteins were purified from clarified lysate using IMAC and gel filtration.

### Generation of Anti-BTN2A1 Monoclonal Antibodies

A human antibody phage display library was used to screen for antibody clones with specificity for BTN2A1. Screening consisted of three rounds of selection for binding to 50 nM recombinant soluble C-terminally His-tagged BTN2A1 ectodomain immobilised on streptavidin-coated paramagnetic beads (Dynal), with pre-adsorption of non-specific binders on an unrelated control His-tagged protein also immobilised on streptavidin-coated beads. After extensive washing, bound phage were eluted and amplified overnight by infection of exponentially growing bacterial cultures (TG1; Stratagene). Purified phage were then used for a subsequent round of panning. After three rounds, bound phage were eluted and 190 clones were randomly picked and tested by ELISA for binding to BTN2A1 immobilised in a microplate. Sequencing of positive clones revealed a total of 52 individual antibody clones, of which 45 were then sub-cloned into a mammalian expression vector for expression in Expi293F™ cells (ThermoFisher) and purification on MabSelect SuRe resin (GE Lifesciences) as full-length IgG molecules which comprised a human IgG4 Fab region and murine IgG2a Fc region. Isotype control clone BM4 contained the same Fc region, except for a mouse Fab region with an irrelevant specificity.

TABLE 2

| | | | |
|---|---|---|---|
| Single-cell | SEQ ID NO: 9 | TRDV2_External | TGGGCAGGAGTCATGTCAG |
| PCR round 1 | SEQ ID NO: 10 | TRDC_Rev1 | GCAGGATCAAACTCTGTTAT |
| | | | CTTC |
| | SEQ ID NO: 11 | TRGV9_External | GGCTCTGTGTGTATATGGTG |
| | | | C |
| | SEQ ID NO: 12 | TRGC_Rev1 | CTGACGATACATCTGTGTTCT |
| | | | TTG |
| Single-cell | SEQ ID NO: 13 | TRDV2_Fwd_soluble | ATACCGGTGCCATTGAGTTG |
| PCR round 2 | | | GTGCCT |
| | SEQ ID NO: 14 | TRDC_Rev_soluble | TGTTCCGGATATCCTTGGGG |
| | | | TAGAATTCCTTCA |
| | SEQ ID NO: 15 | TRDV9_Fwd_soluble | ATACCGGTGCAGGTCACCTA |
| | | | GAGCAAC |
| | SEQ ID NO: 16 | TRDC_Rev_soluble | CAGCAATTGAAGGAAGAAA |
| | | | AATAGTGGGCTTG |
| Site-directed | SEQ ID NO: 17 | E28Aδ_Fwd | ATGAAGGGCGcAGCCATCGG |
| mutagenesis | | | C |
| | SEQ ID NO: 18 | E28Aδ_Rev | GCTACACCGCAGTGTGGC |
| | SEQ ID NO: 19 | R51Aδ_Fwd | CTTCATCTACgcAGAGAAGGA |
| | | | CATCTACGG |
| | SEQ ID NO: 20 | R51Aδ_Rev | GTCATGGTGTTGCCCTGG |
| | SEQ ID NO: 21 | L97Aδ_Fwd | CTGTGACACAgcTGGAATGG |
| | | | GCGGCGAG |
| | SEQ ID NO: 22 | L97Aδ_Rev | GCGCAGTAGTAGCTGCCC |
| | SEQ ID NO: 23 | E5Aγ_Fwd | GGACATCTGGcACAGCCCCA |
| | | | G |
| | SEQ ID NO: 24 | E5Aγ_Rev | AGCGCCATACACACACAG |
| | SEQ ID NO: 25 | R20Aγ_Fwd | CAAGACCGCCgcACTGGAAT |
| | | | GC |
| | SEQ ID NO: 26 | R20Aγ_Rev | CTCAGTGTCTTGGTGCTG |
| | SEQ ID NO: 27 | E22Aγ_Fwd | GCCAGACTGGcATGCGTGGT |
| | | | G |
| | SEQ ID NO: 28 | E22Aγ_Rev | GGTCTTGCTCAGTGTCTTGG |
| | SEQ ID NO: 29 | T29Aγ_Fwd | GTCCGGCATCgCAATCAGCG |
| | | | C |
| | SEQ ID NO: 30 | T29Aγ_Rev | ACCACGCATTCCAGTCTGG |
| | SEQ ID NO: 31 | Y54Aγ_Fwd | GTCCATCAGCgcCGATGGCAC |
| | | | C |
| | SEQ ID NO: 32 | Y54Aγ_Rev | ACCAGGAACTGGATCACTTC |
| | SEQ ID NO: 33 | T57Aγ_Fwd | CTACGATGGCgCCGTGCGGA |
| | | | A |
| | SEQ ID NO: 34 | T57Aγ_Rev | CTGATGGACACCAGGAACTG |
| | | | G |
| | SEQ ID NO: 35 | K60Aγ_Fwd | CACCGTGCGGgcAGAGAGCG |
| | | | GC |
| | SEQ ID NO: 36 | K60Aγ_Rev | CCATCGTAGCTGATGGACAC |
| | SEQ ID NO: 37 | S62Aγ_Fwd | GCGGAAAGAGgcCGGCATCC |
| | | | CTTC |
| | SEQ ID NO: 38 | S62Aγ_Rev | ACGGTGCCATCGTAGCTG |
| | SEQ ID NO: 39 | S66Aγ_Fwd | CGGCATCCCTgCTGGCAAGTT |
| | SEQ ID NO: 40 | S66Aγ_Rev | CTCTCTTTCCGCACGGTG |
| | SEQ ID NO: 41 | E70Aγ_Fwd | GGCAAGTTCGcGGTGGACAG |
| | | | AATC |
| | SEQ ID NO: 42 | E70Aγ_Rev | AGAAGGGATGCCGCTCTC |
| | SEQ ID NO: 43 | E76Aγ_Fwd | AGAATCCCCGcGACAAGCAC |
| | | | C |
| | SEQ ID NO: 44 | E76Aγ_Rev | GTCCACCTCGAACTTGCC |
| | SEQ ID NO: 45 | H85Aγ_Fwd | ACTGACCATCgcCAACGTGGA |
| | | | AAAGCAG |
| | SEQ ID NO: 46 | H85Aγ_Rev | GTGCTGGTGCTTGTCTCG |
| | SEQ ID NO: 47 | N86Aγ_Fwd | GACCATCCACgcCGTGGAAA |
| | | | AGCAG |
| | SEQ ID NO: 48 | N86Aγ_Rev | AGTGTGCTGGTGCTTGTC |
| | SEQ ID NO: 49 | E88Aγ_Fwd | CACAACGTGGcAAAGCAGGA |
| | | | TATC |
| | SEQ ID NO: 50 | E88Aγ_Rev | GATGGTCAGTGTGCTGGT |
| | SEQ ID NO: 51 | Q90Aγ_Fwd | CGTGGAAAAGgcGGATATCG |
| | | | CC |
| | SEQ ID NO: 52 | Q90Aγ_Rev | TTGTGGATGGTCAGTGTG |
| | SEQ ID NO: 53 | K108Aγ_Fwd | AGAGCTGGGCgcGAAAATCA |
| | | | AGGTGTTCG |
| | SEQ ID NO: 54 | K108Aγ_Rev | TGTTGGGCTTCCCACAGG |
| CRISPR/Cas9 | SEQ ID NO: 55 | CRISPR 2 top | TCACAAAGGTGGTTCTTCCT |
| | SEQ ID NO: 56 | CRISPR 2 bottom | AGGAAGAACCACCTTTGTGA |
| | | | CGGTG |
| | SEQ ID NO: 57 | CRISPR 4 top | CAATAGATGCATACGGCAAT |
| | SEQ ID NO: 58 | CRISPR 4 bottom | ATTGCCGTATGCATCTATTGC |
| | | | GGTG |
| | SEQ ID NO: 59 | sc-404202 A | GGCACTTACGAGATGCATAC |
| | SEQ ID NO: 60 | sc-404202 B | GAGAGACATTCAGCCTATAA |
| | SEQ ID NO: 61 | sc-404202 C | ACCATCAGAAGTTCCCTCCT |
| | SEQ ID NO: 62 | 2A1 CRISPR1 | GTGACCTATGAACTCAGGAG |
| | | MiSeqF | TCCTTGAGTGACGGGAGAGG |
| | | | TT |
| | SEQ ID NO: 63 | 2A1 CRISPR1 | CTGAGACTTGCACATCGCAG |
| | | MiSeqR | CTCCTTTTGGACAGTGCTGGT |
| | SEQ ID NO: 64 | 2A1 CRISPR2 | GTGACCTATGAACTCAGGAG |
| | | MiSeqF | TCCCTTTGTTGAACAGCCCA |
| | | | GT |
| | SEQ ID NO: 65 | 2A1 CRISPR2 | CTGAGACTTGCACATCGCAG |
| | | MiSeqR | CTAGGACCTGCCTTCTTGGA |
| | | | A |
| | SEQ ID NO: 66 | 2A1 CRISPR3 | GTGACCTATGAACTCAGGAG |
| | | MiSeqF | TCCCGAGAAAAATGCTGAGG |
| | | | AC |
| | SEQ ID NO: 67 | 2A1 CRISPR3 | CTGAGACTTGCACATCGCAG |
| | | MiSeqR | CAATGGGCCTGAGGTTAGGA |
| | | | G |
| | SEQ ID NO: 68 | 2A1 CRISPR4 | GTGACCTATGAACTCAGGAG |
| | | MiSeqF | TCAGAAAGCAGGAGAGCAG |
| | | | GTG |
| | SEQ ID NO: 69 | 2A1 CRISPR4 | CTGAGACTTGCACATCGCAG |
| | | MiSeqR | CTTGCACACGTTCTTTCTCCA |

### Production of Anti-BTN3A Antibodies

DNA constructs encoding anti-BTN3A antibody variable domains (clones 20.1 and 103.2; described in Palakodeti et al. (2012)) were synthesized (ThermoFisher) and cloned into mammalian expression vectors containing a mouse IGHV signal peptide and IgG1 constant regions. Antibodies were expressed in Expi293F™ cells as above and purified using Protein G column chromatography 60(GE), followed by buffer-exchange into PBS.

### Enzyme-Linked Immunosorbent Assay

Purified recombinant proteins (0.2-20 μg/ml) were immobilized in microplate wells in PBS buffer overnight at 4° C. Non-specific binding was then blocked by incubation in PBS containing 0.05% tween 20 plus 5% skim milk powder or 0.5% (w/v) bovine serum albumin (BSA). The wells were then incubated for 60 minutes at room temperature in the presence of antibodies at 2-5 μg/mL in PBS/0.05% tween-20/2% skim milk powder or 0.5% BSA, followed by washing in PBS/0.05% tween-20. Plates were then incubated with HRP-labelled sheep anti-mouse IgG secondary antibody (Chemicon), or goat anti-mouse IgG secondary antibody (Millipore) followed by detection using 3,3′,5,5′-tetramethylbenzidine substrate (Sigma) and absorbance was measured at 450 nm using a plate reader.

### Generation of CRISPR/Cas9-Mediated Knockout Cell Lines

For BTN2A1 knockout lines two gRNAs (BTN2A1^{null1}; 5′-TCACAAAGGTGGTTCTTCCT-3′ (SEQ ID NO: 55) and BTN2A1^{null2}: 5′-CAATAGATGCATACGGCAAT-3′) (SEQ ID NO: 57) were cloned into GeneArt® CRISPR Nuclease Vector Kit (Life Technologies) according to the manufacturer's protocol and sequence-verified by Sanger sequencing. Cells were transfected using Lipofectamine 2000 and sorted after 48 h based on orange fluorescent protein expression. Cells were cultured and stained with anti-BTN2A1 (clone Hu34C) and the negative fraction sorted. For BTN3A1 knockout lines, a BTN3A1 CRISPR/Cas9 KO Plasmid kit (Santa Cruz Biotechnology) containing three specific gRNA sequences was used (5′-GGCACTACGAGATGCATAC-3′ (SEQ ID NO:59), 5′-GAGAGACATICAGCCTATAA-3′ (SEQ ID NO: 60), 5′-ACCATCAGAAGTCCCTCCT-3′ (SEQ ID NO: 61)). Cells were transfected using Lipofectamine 3000 (ThermoFisher) and sorted after 48 h based on green fluorescent protein. Sorted cells were cultured and stained with anti-BTN3A1 (clone 103.2) and negative fraction sorted and cultured.

### Jurkat Assays

LM-MEL-62 or LM-MEL-75 APCs at 2.5×10⁴ cells/well in a 96-well plate and incubated overnight. Then 2×10⁴ G115 mutant γδTCR-expressing J.RT3-T3.5 (ATCC® TIB-153™) (Jurkat) cells ±zoledronate, HMBPP or IPP were added for 20 h. CD69 expression was then measured by flow cytometry on GFP⁺ Jurkat cells. A panel of nineteen single-residue alanine (Ala) mutants, each within in the Vγ9 or Vδ2 domains of the Vγ9Vδ2⁺ G115 TCR were generated by site-directed mutagenesis using the primers listed in Table 2). Primers (IDT) were phosphorylated (PNK, NEB) followed by 25 cycles of PCR using KAPA HiFi master mix (KAPA Biosystems) using WT G115 in pMIG as template, and PCR product was digested with DpnI (NEB) and ligated with T4 DNA ligase (NEB). Construct sequences were then verified by Sanger sequencing prior to transfections.

To examine the capacity of G115 TCR mutants to bind to BTN2A1 tetramer, HEK-293T cells were transfected with individual γ-chain or δ-chain mutants, plus the corresponding WT δ- or γ-chain, respectively, as well as a pMIG construct encoding 2A-linked human CD3γδεζ, at a 1:3 ratio with FuGENE® HD (Promega) in OptiMEM™ (Gibco, Thermo-Fisher). 48 h following transfection, HEK293T cells were resuspended by pipetting, and stained for CD3ε expression and PE-labelled BTN2A1 tetramer or control PE-conjugated streptavidin. The median fluorescence intensity (MFI) of BTN2A1 tetramer interacting with mutant G115 TCRs was examined on gated CD3⁺ GFP⁺ HEK293T cells, by flow cytometry.

The capacity of G115 mutants to respond to pAg stimulation was assessed by transducing J.RT3-T3.5 Jurkat cells with G115 mutant TCRs. HEK-293T cells were transfected with each particular γ-chain or δ-chain mutant, plus the corresponding wild-type δ- or γ-chain respectively, along with human CD3, pVSV(-G) and pEQ-Pam3(-E), mixed at 1:3 ratio with FuGENE® HD in OptiMEM™. After 24 h, viral supernatants were collected and filtered through a 0.45 μm CA syringe filter, then incubated with JRT3-T3.5 Jurakt cells for 12h. This process was repeated twice a day for four days. CD3⁺GFP⁺ Jurkat cells were purified by FACS (BD FACSAria™ III) and examined for their capacity to respond to pAg presented by wild-type LM-MEL-75 APCs as described above.

To measure G115 γδTCR-expressing Jurkat cell reactivity to anti-BTN3A1 (clone 20.1) mAb, 2.5×10⁴ LM-MEL-75 APC cells were pre-incubated with functional grade 20.1 (10 μg/ml, Biolegend), or matched isotype control for 30 minutes at room temperature and later plated in a flat-bottom 96 well plate. Once the APCs had adhered, 2.5×10⁴ Jurkat cells were added making a final antibody concentration of 5 μg/ml. After 24 h coculture the level of CD69 on CD3⁺GFP⁺ Jurkat cells was determined by flow cytometry.

### Surface Plasmon Resonance

SPR experiments were conducted at 25° C. on a Proteon XPR36 instrument (Bio-Rad) using 10 mM HEPES-HCl (pH 7.4), 300 mM NaCl and 0.005% Tween-20 buffer. γδTCR ectodomains were directly immobilized to 260 resonance units (RU) on a Biacore sensor chip SA pre-immobilized with streptavidin. Soluble butyrophilins were serially diluted (200-3.1 μM) and simultaneously injected over test and control surfaces at a rate of 30 μl/min. After subtraction of data from the control flow cell (streptavidin alone) and blank injections, interactions were analyzed using Biacore T200 evaluation software (GE Healthcare) and Prism version 8 (GraphPad), and equilibrium dissociation constants (K_{D}) were derived at equilibrium.

### Isothermal Titration Calorimetry

ITC experiments were conducted on a MicroCal ITC200 instrument (GE Healthcare) at 25° C. BTN2A1 or BTN3A1 B30.2 domains were buffer exchanged into PBS, and adjusted to a final concentration of 100 μM. HMBPP (Cayman Chemical) or IPP were adjusted to a final concentration of 2 mM and serially injected into the cell in 2 μl increments, following an initial 0.4 μl injection that was discarded from the analysis. Data were analysed with Microcal Origin software.

### Confocal Microscopy

LM-MEL-75 WT, BTN2A1^{null}, BTN3A1^{null} cells were cultured overnight in RPMI-1640 (Thermo-Fisher) supplemented with 10% (v/v) FCS (JRH Biosciences), penicillin (100 U/ml), streptomycin (100 μg/ml), Glutamax (2 mM), sodium pyruvate (1 mM), nonessential amino acids (0.1 mM) and HEPES buffer (15 mM), pH 7.2-7.5 (all from Invitrogen Life Technologies), plus 50 μM 2-mercaptoethanol (Sigma-Aldrich) and allowed to adhere to chamber well slides (Lab-Tek, Thermo-Fisher). The following day cells were washed and incubated with human Fc receptor block (Miltenyi Biotec) diluted with OptiMEM™ (Thermo-Fisher) on ice for 20 min. Cells were washed and stained with anti-BTN2A1-AF647 (clone 259), anti-BTN3A-PE (clone 103.2) and anti-pan-HLA class I-AF488 (clone W6/32, BioLegend) diluted in OptiMEM™ on ice for 20 min. Cells were fixed with 1% paraformaldehyde (Electron Microscopy Sciences) in PBS for 20 min then mounted with ProLong Gold AntiFade (Thermo-Fisher) and covered with a #1 coverslip (Menzel-Gläser) overnight. Each reagent was titrated to determine the optimal dilution factor. Z-stack, single tile images with 76.9 nm lateral and 400 nm axial voxel size and 1024×1024 voxel density were acquired on a LSM780 laser scanning confocal microscope with an inverted 20× (0.8 NA) objective and Zen software (Zeiss). Fluorochromes were excited with 488-, 561- and 633-nm laser lines. Images were deconvoluted with Huygens Professional (Scientific Volume Imaging) and analyzed with Imaris (Oxford Instruments) software. Regions of interests defining the imaged cells were made based on the brightfield channel and the Imaris Coloc module was used to calculate Pearson correlation coefficients of voxels with intensity thresholds set for each analysed channel based on negative controls for each stain.

BTN2A1 is a ligand for Vγ9⁺ γδTCR

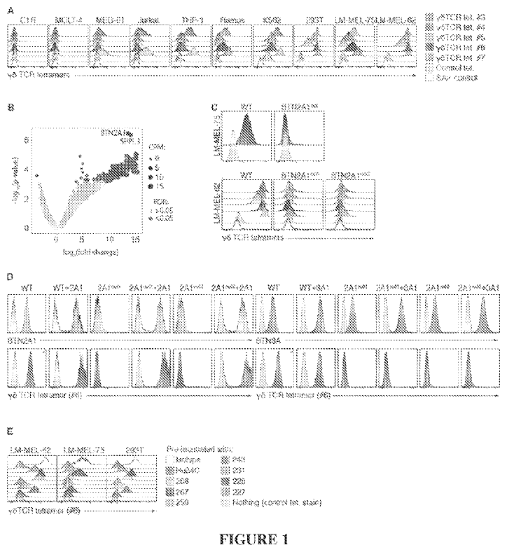

To identify candidate ligands for Vγ9Vδ2⁺ γδ TCRs, the inventors generated soluble Vγ9Vδ2⁺ TCR tetramers derived from pAg-reactive γδ T cells (FIG. 8), and used them to stain a diverse panel of human cell lines. This revealed clear staining of some lines including HEK-293T, but not others including the B cell line C1R (FIG. 1A). In particular, a melanoma cell line LM-MEL-62 was strongly stained (A. Behran et al. (2013)) (FIG. 1A). Using a genome-wide knockdown screen (FIG. 9), the most significant guide RNA (gRNA) responsible for Vγ9Vδ2⁺ TCR-tetramer reactivity was BTN2A1, with a >13-fold enrichment compared to the controls (FIG. 1A and FIG. 9). BTN2A1 is a poorly characterized member of the butyrophilin family, found in humans but not mice. Like BTN3A1, it consists of two extracellular domains (IgV and IgC) and an intracellular B30.2 domain. Apart from one study suggesting it may interact with the C-type lectin receptor CD209 (DC-SIGN) in a glycosylation-dependent manner (G. Malcherek et al (2007)), BTN2A1 is generally considered an orphan receptor. To further investigate the significance of this finding, the inventors confirmed a loss of reactivity to Vγ9Vδ2⁺ TCR tetramers in two independent LM-MEL-62 BTN2A1 mutant lines (BTN2A1^{null1} and BTN2A1^{null2}), with similar results also from a distinct LM-MEL-75 BTN2A1 mutant cell line (FIG. 1C and FIG. 10). This was independent of BTN3A1 expression, which was essentially unchanged between parental LM-MEL-62 and BTN2A1^{null} lines (FIG. 1C and FIG. 10A). Additionally, Vγ9Vδ2 TCR tetramer reactivity of BTN3A1^{null} lines was comparable to parental lines (FIG. 10B). Reintroduction of BTN2A1 into either LM-MEL-62 BTN2A1^{null1} or BTN2A1^{null2} cells restored Vγ9Vδ2 TCR tetramer reactivity, whilst transfection with BTN3A1 had no effect (FIG. 1D). Thus, BTN2A1 expression is essential for Vγ9Vδ2⁺ TCR tetramer reactivity.

The inventors next generated a panel of BTN2A1-reactive mAbs, which exhibited varying degrees of cross-reactivity to BTN2A2 (87% ectodomain homology) but not to BTN3A2 (45% ectodomain homology) (FIG. 11A-C). These mAbs stained parental LM-MEL-62 but most failed to bind to LM-MEL-62 BTN2A1^{null} lines, confirming their reactivity to BTN2A1 (FIG. 11D-E). Most of the anti-BTN2A1 clones blocked, or partially blocked, Vγ9Vδ2 TCR tetramer staining on LM-MEL-62, LM-MEL-75, and 293T cells (FIG. 1E), suggesting that BTN2A1 is a ligand for the Vγ9Vδ2⁺ γδTCR.

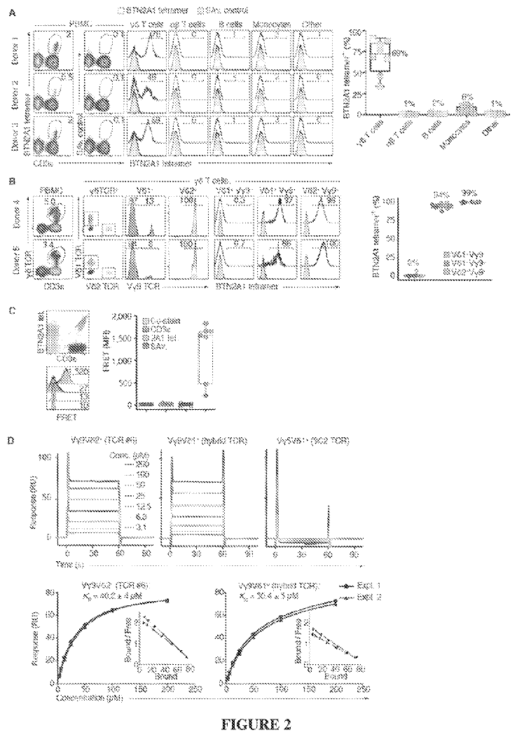

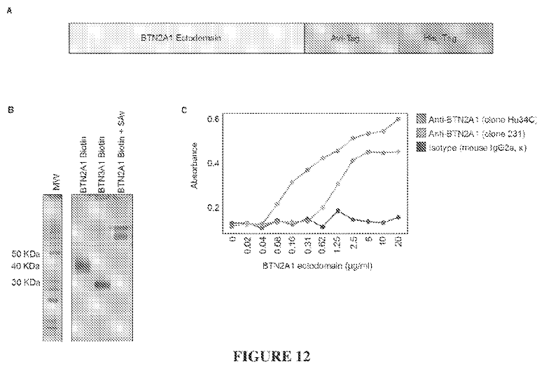

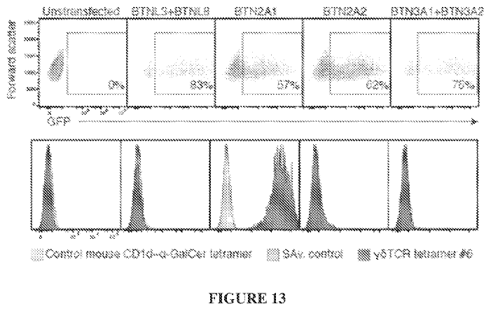

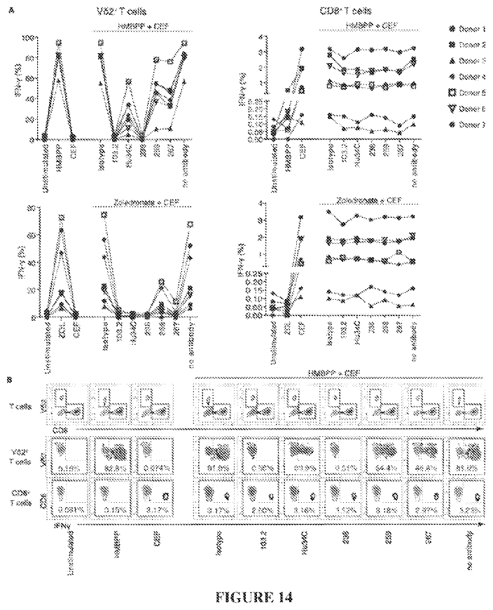

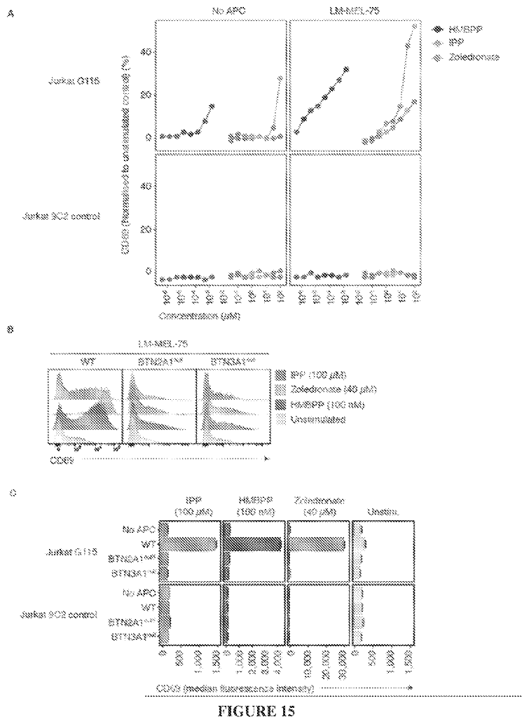

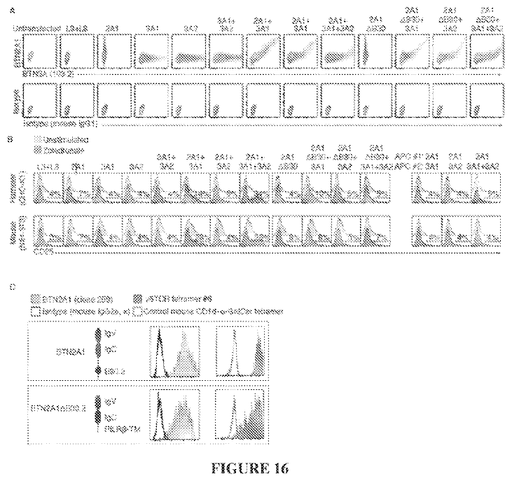

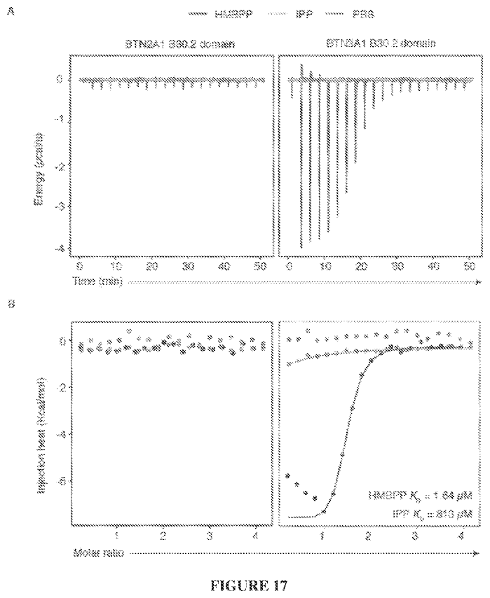

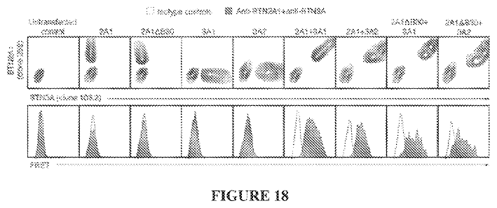

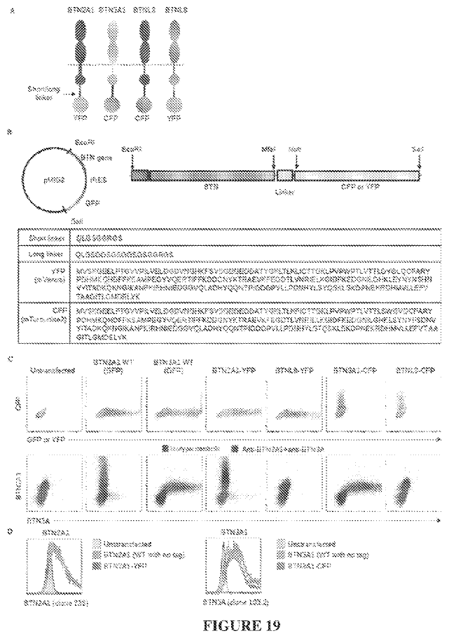

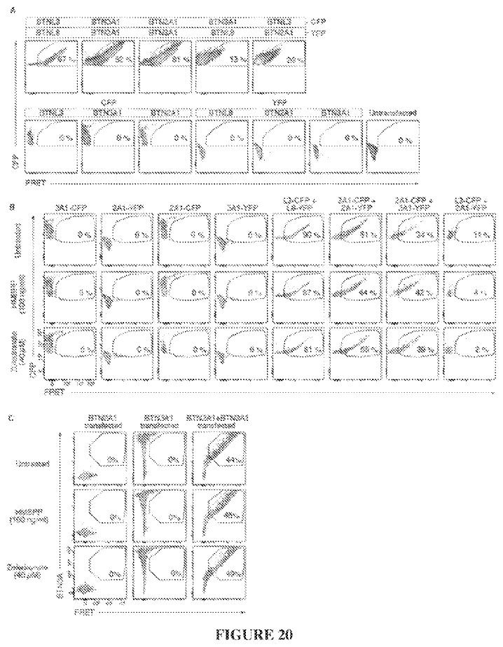

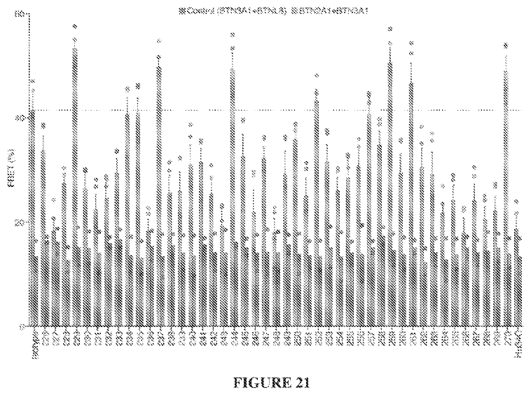

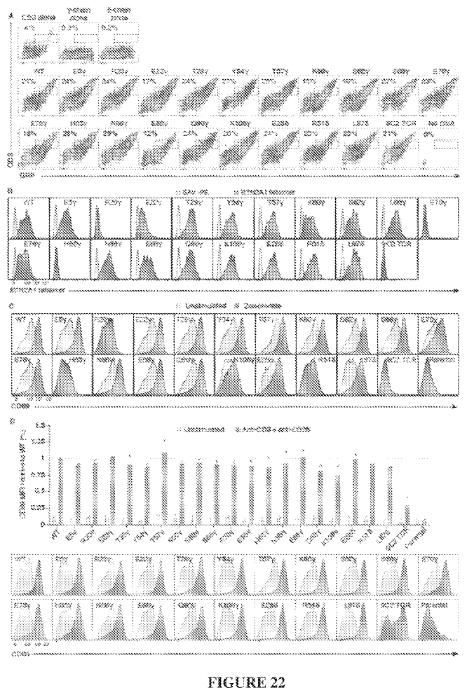

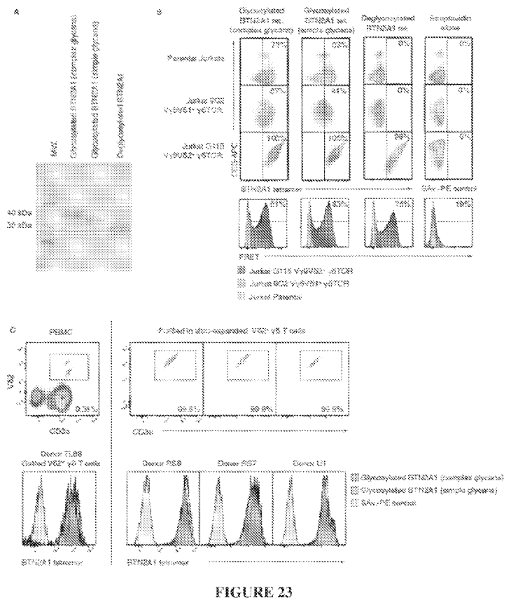

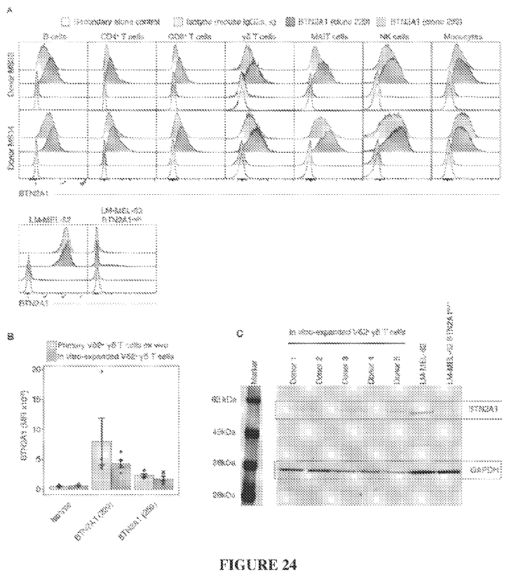

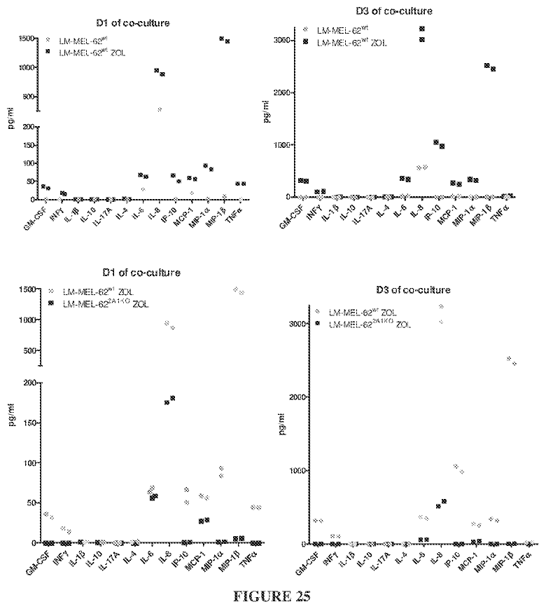

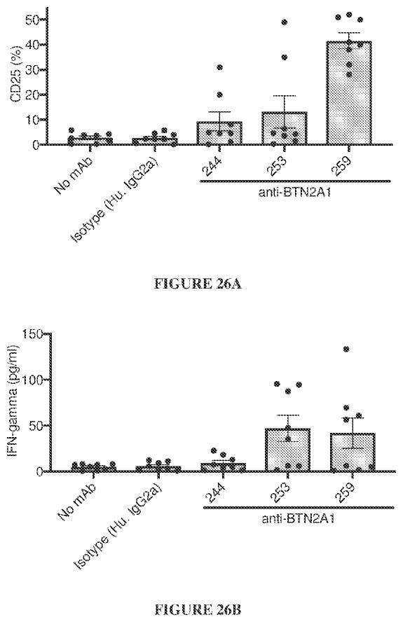

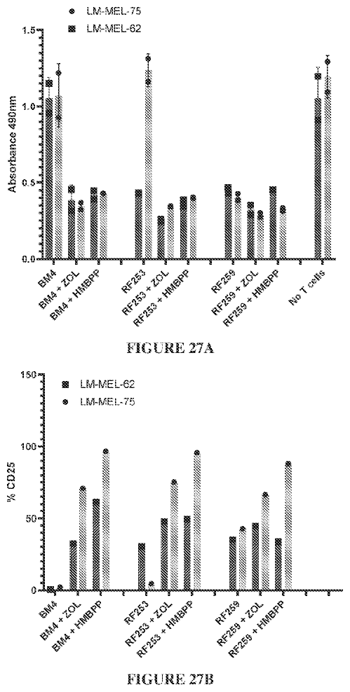

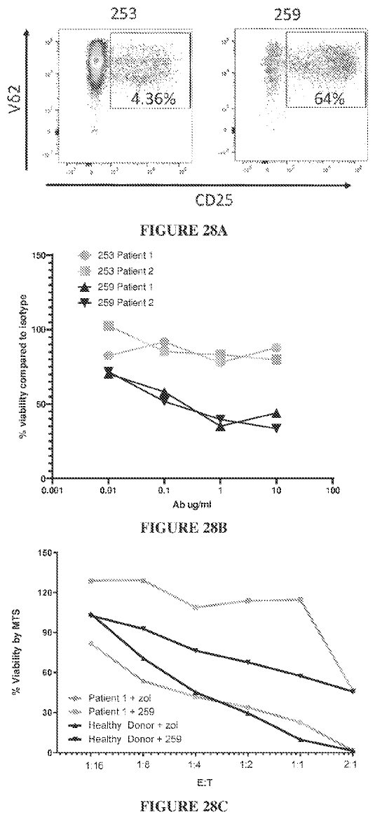

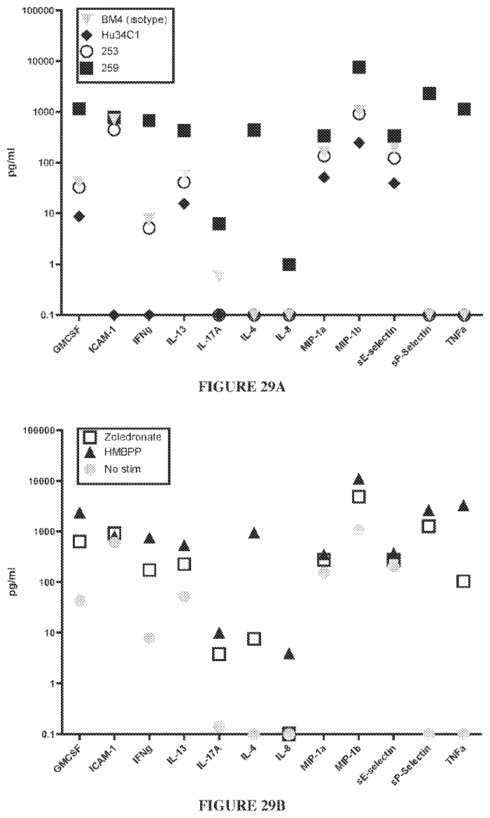

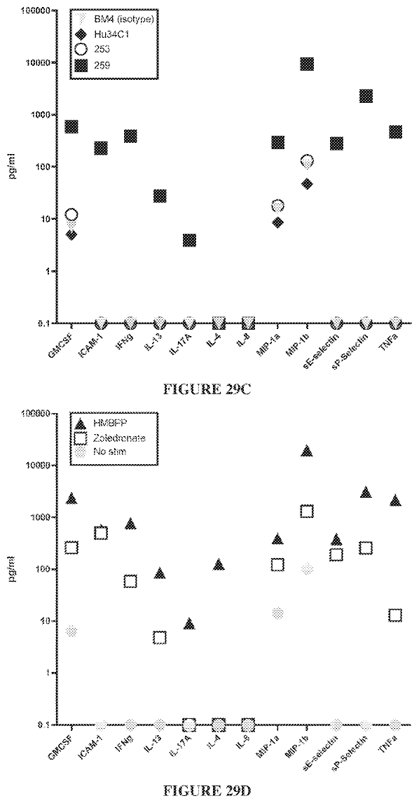

To explore whether BTN2A1 selectively binds to Vγ9Vδ2⁺ γδ T cells, the inventors produced fluorescent BTN2A1 ectodomain tetramers (FIG. 12), which stained a subset of CD3⁺ T cells within PMBCs, but no other cell type (FIG. 2A). The BTN2A1 tetramer⁺ cells were γδTCR⁺, but not αβTCR⁺ (FIG. 2A). BTN2A1 tetramer labelled essentially all Vγ9⁺Vδ2⁺ and Vγ9⁺Vδ1⁺ γδ T cells, but no Vγ9⁻Vδ1⁺ γδ T cells, suggesting that the Vγ9 domain of the TCR γ-chain is associated with reactivity (FIG. 2B). Furthermore, Forster resonance energy transfer (FRET) between fluorescent BTN2A1 tetramer and anti-CD3ε mAb (P. Batard et al (2002)) indicated that BTN2A1 tetramer was binding within ˜10 nm on the γδTCR (FIG. 2C). To directly assess whether BTN2A1 binds Vγ9⁺ γδTCR, the inventors performed surface plasmon resonance (SPR) to measure interactions between soluble BTN2A1 and γδTCR ectodomains. Consistent with the pattern of BTN2A1 tetramer reactivity amongst primary γδ T cells, soluble BTN2A1 TCR #6 (Vγ9Vδ2⁺) with an affinity of K_{D}=40 μM, similar to what is observed for classical αβ T cells (M. E. Birnbaum et al (2014)). It also bound a “hybrid” γδTCR that co-expressed the TCR #6 γ-chain paired with an irrelevant Vδ1⁺ γ-chain with comparable affinity (50 μM). However, BTN2A1 did not bind to a γδTCR comprised of a Vγ5⁺ γ-chain paired with the Vδ1⁺ δ-chain (FIG. 2D). Lastly, the inventors tested whether other butyrophilin family members could bind to Vγ9Vδ2 TCR. BTN2A2 exhibited only very weak binding, and BTN3A1+BTN3A2 and BTNL3+BTNL8 transfected cells did not bind Vγ9Vδ2 TCR tetramers (FIG. 13). Thus, BTN2A1 is a ligand for Vγ9⁺ γδTCR.

BTN2A1 is important for γδ T cell responses to pAg

The inventors next determined if BTN2A1 is important in pAg-mediated γδ T cell responses. As expected, PBMCs cultured with the aminobisphosphonate compound zoledronate, which induces accumulation of the pAg IPP (A. J. Roelofs et al. (2009)), resulted in Vδ2⁺ but not Vδ1⁺ γδ T cell induction of CD25, downregulation of surface CD3 (FIG. 3A), and IFN-γ and TNF production (FIG. 3B). These indicators of TCR-dependent activation were significantly inhibited by anti-BTN2A1 mAb clone Hu34 and, to lesser extents, by clones 259 and 267, compared to isotype control mAb-treated samples. Next, purified in vitro pre-expanded Vγ9Vδ2⁺ T cells were cultured with parental or BTN2A1^{null} LM-MEL-62 cells as APCs. Robust Vδ2⁺ T cell responses to zoledronate, in terms of CD25 upregulation and CD3 downregulation, were observed in the presence of parental LM-MEL-62 APCs. However, both BTN2A1^{null1} and BTN2A1^{null2} APCs failed to promote γδ T cell activation above control cultures without APCs (FIG. 3C). Similarly, proliferative expansion of Vδ2+ γδ cells was diminished when BTN2A1^{null1} APCs were used (FIG. 3D). The inventors also observed a γδ T cell-mediated, zoledronate-dependent, killing of parental LM-MEL-62 tumor cells that was not observed with BTN2A1^{null1} cells, suggesting that BTN2A1 is important for Vγ9Vδ2⁺ T cell cytotoxicity of tumor targets (FIG. 3D). These data indicate that BTN2A1 is important for γδ T cell responses to endogenous forms of pAg.

Vγ9Vδ2⁺ γδ T cells can self-present high affinity foreign forms of pAg such as microbial HMBPP in the absence of APCs (C. T. Morita et al. (1995)). BTN2A1 was also indispensable in this setting since purified in vitro pre-expanded Vδ2⁺ T cells failed to upregulate CD25 and produce IFN-γ in the presence of neutralizing anti-BTN2A1 mAb (clones Hu34C, 227, 236, and 266) (FIG. 3E). Clone 267 was only a partial inhibitor of HMBPP-induced activation (FIG. 3E). Importantly, these mAbs did not inhibit anti-CD3 plus anti-CD28-mediated activation (FIG. 3E) nor did they block primary CD8⁺ αβ T cell activation mediated by a mixture of viral peptides derived from cytomegalovirus, Epstein-Barr virus and influenza epitopes (“CEF” peptide, FIG. 14). Thus, these BTN2A1 mAbs are specific antagonists of both self and foreign forms of pAg-driven T cell immunity. Taken together, BTN2A1 plays an important role in pAg-mediated cytokine production, activation, proliferation, and tumor cytotoxicity by human Vγ9Vδ2⁺ γδ T cells.

BTN2A1 Co-Operates with BTN3A1 to Elicit pAg Responses by γδ T Cells

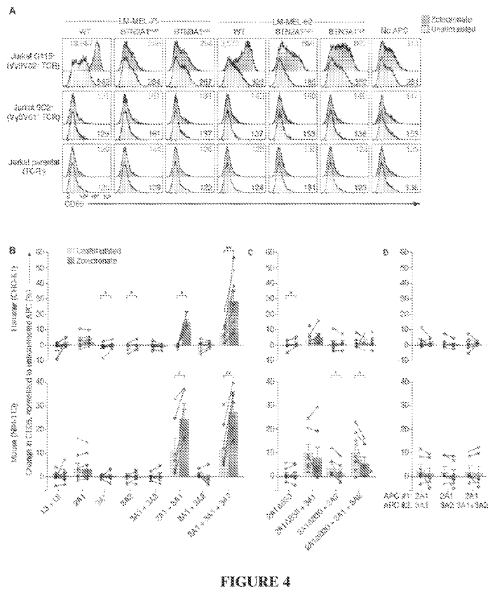

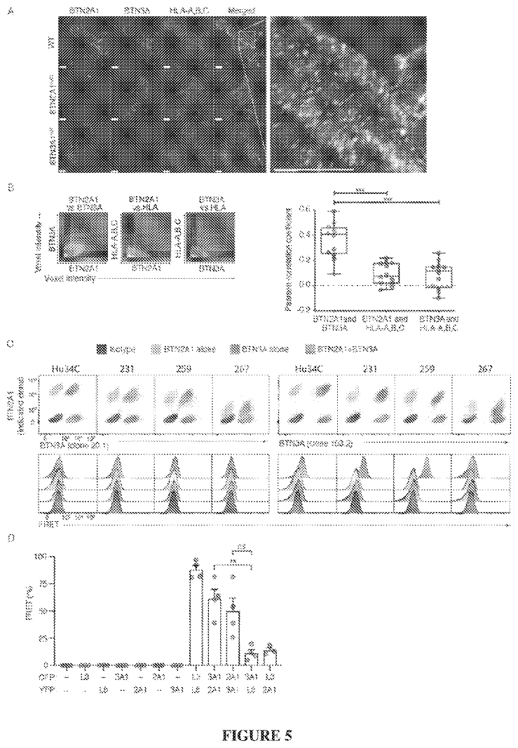

The inventors next determined if BTN2A1-dependent pAg responses are specifically mediated via γδTCR signaling. Following co-culture with either parental LM-MEL-75 or LM-MEL-62 APCs, J.RT3-T3.5 (Jurkat) T cells expressing the prototypical “G115” Vγ9Vδ2⁺ TCR clonotype (T. J. Allison et al. (2001)) upregulated CD69 in response to zoledronate; however, BTN2A1^{null} and BTN3A1^{null} APCs largely failed to induce pAg reactivity (FIG. 4A). Untransduced (parental) Jurkat cells or those expressing an irrelevant γδTCR (clone 9C2; A. P. Uldrich et al. (2013)) also failed to respond to pAg. Similar results were obtained using HMBPP and IPP (FIG. 15A-C), indicating that BTN2A1 and BTN3A1 are both required to specifically mediate pAg responses in a Vγ9Vδ2⁺ γδTCR-dependent manner.

Although BTN3A1 is essential for pAg-mediated responses, forced BTN3A1 overexpression fails to confer pAg-driven γδ T cell-stimulatory capacity to hamster and mouse APCs, indicating a requirement for other factors (A. Sandstrom et al. (2014); F. Riano et al. (2014). The inventors found that both hamster and mouse APCs transfected with BTN2A1 and BTN3A1 in combination, but not alone, were capable of pAg-dependent activation of γδ T cells (FIG. 4B and FIG. 16A-B). Although another butyrophilin molecule, BTN3A2, was not necessary for this response, it moderately enhanced activation of γδ T cells when combined with BTN2A1 and BTN3A1, consistent with its potential role in increasing BTN3A1 activity (P. Vantourout et al. (2018)). A modified BTN2A1 construct with irrelevant transmembrane and intracellular domains derived from mouse paired immunoglobulin-like type 2 receptor beta, termed BTN2A1ΔB30, was also tested. This was still expressed on the cell surface and bound Vγ9Vδ2⁺ TCR tetramer (FIG. 16C), but it did not confer pAg-presenting capacity (FIG. 4C). Thus, in addition to the role of its extracellular domain in binding Vγ9⁺ γδTCR, the intracellular or transmembrane domain of BTN2A1 may also be important for pAg-mediated activation of Vγ9Vδ2⁺ γδ T cells. This did not appear to be due to the intracellular B30.2 domain of BTN2A1 directly binding purified pAgs (HMBPP or IPP) because no clear interaction between these molecules was detected using isothermal titration calorimetry (FIG. 17), in contrast to the clear interaction between the BTN3A1 B30.2 domain with pAg, as expected (A. Sandstrom et al. (2014), S. Gu et al., (2017), M. Salim et al (2017)).

Lastly, the inventors tested whether BTN2A1 and BTN3A1 induce pAg-mediated activation when expressed on the same cell (in cis) or on separate cells (in trans). BTN2A1 APC mixed with either BTN3A1⁺ APCs, or BTN3A1⁺BTN3A2⁺ APCs, failed to elicit γδ T cell responses to pAg (FIG. 4D), suggesting that these molecules must be expressed on the same APC to mediate pAg-induced activation of γδ T cells.

BTN2A1 Associates with BTN3A Molecules on the Cell Surface

The requirement for BTN2A1 and BTN3A1 co-expression in cis raised the possibility that they associate with each other. Parental LM-MEL-75 cells stained with anti-BTN2A1 and anti-BTN3A1/3A2/3A3 (“BTN3A molecules”) mAbs showed a similar staining pattern for BTN2A1 and BTN3A molecules on the cell surface (FIG. 5A-C). The Pearson correlation coefficients indicated a significant overlap between the staining of BTN2A1 and BTN3A molecules, compared to the overlap of either with an irrelevant control (HLA-A,B,C). Thus, BTN2A1 and BTN3A molecules appear to be associated on the plasma membrane (FIG. 5B).

Furthermore, co-staining of LM-MEL-75 cells with anti-BTN2A1 (clone 259) and anti-BTN3A (clone 103.2) resulted in a clear FRET signal (FIG. 5C), indicative of colocalization on the cell surface (FIG. 5C). Co-staining with anti-BTN3A (clone 20.1) failed to cause FRET, and likewise, some other anti-BTN2A1 clones (Hu34C and 267) resulted in only weak FRET, which may be because some mAb combinations yield spatially segregated donor and acceptor fluorochromes beyond the 10-nm limit for FRET detection. Similar results were derived using mouse NIH-3T3 fibroblasts transfected with different combinations of BTN molecules (FIG. 18). Interestingly, staining of BTN2A1ΔB30⁺BTN3A1⁺ or BTN2A1ΔB30⁺BTN3A2⁺NIH-3T3 cells with anti-BTN2A1 and anti-BTN3A also resulted in clear FRET. The latter findings suggest that the association between these molecules is independent of the B30.2 domains, since BTN3A2 also lacks a B30.2 domain (FIG. 18).

The inventors next determined whether the intracellular domains of BTN2A1 and BTN3A1 are also associated by generating cyan fluorescent protein (CFP) or yellow fluorescent protein (YFP)-conjugated butyrophilin constructs (FIG. 19). Co-transfection of mouse NIH-3T3 fibroblasts with BTN2A1CFP+BTN3A1^{YFP}, or BTN2A1^{YFP}+BTN3A1^{CFP} resulted in clear FRET signals, similar to the positive controls that are known to associate (butyrophilin-like molecule 3 (BTNL3)^{CFP}+BTNL8^{YFP}) (P. Vantourout et al. (2018)). Little or no FRET was seen in BTN3A1^{CFP}+BTNL8^{YFP} or BTNL3^{CFP}+BTN2A1^{YFP} or single-transfectant controls (FIG. 5D and FIG. 20A). The inventors also tested whether pAg modulated the FRET signal between BTN2A1 and BTN3A1 but did not detect any major changes (FIGS. 20B and 20C); however, anti-BTN2A1 mAb clones with antagonist activity (from FIG. 3D) all strongly disrupted their association (FIG. 21). Thus, both the extracellular and intracellular domains of BTN2A1 and BTN3A1 are closely associated.

### Vγ9Vδ2⁺ γδTCR Co-Recognizes at Least Two Ligands

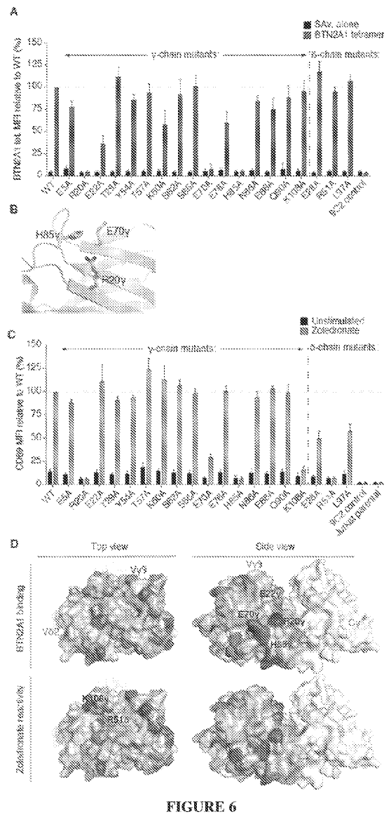

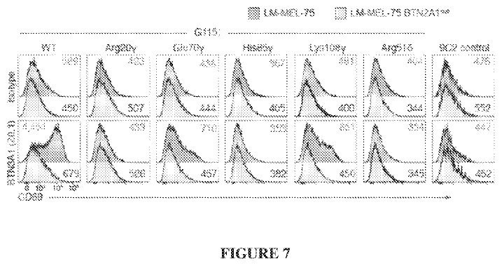

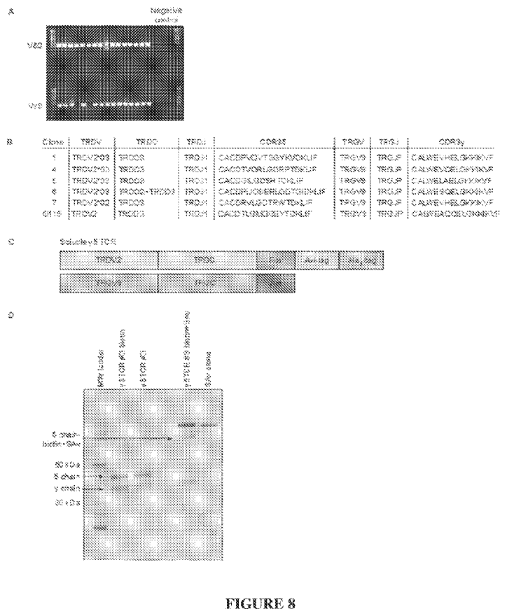

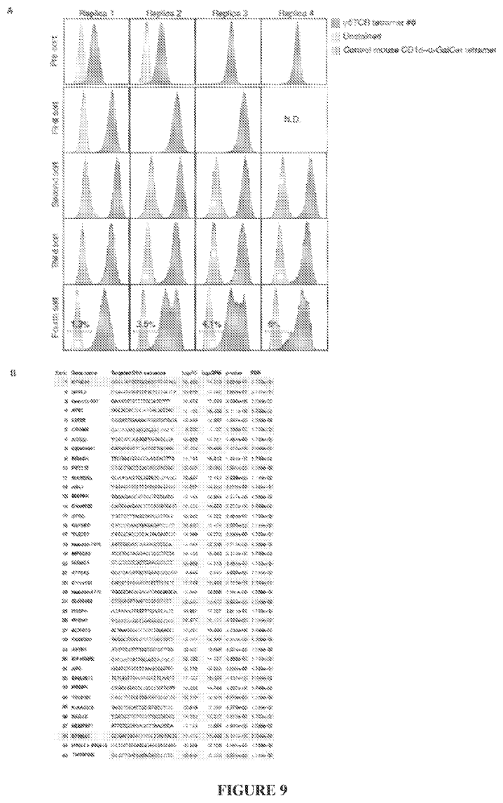

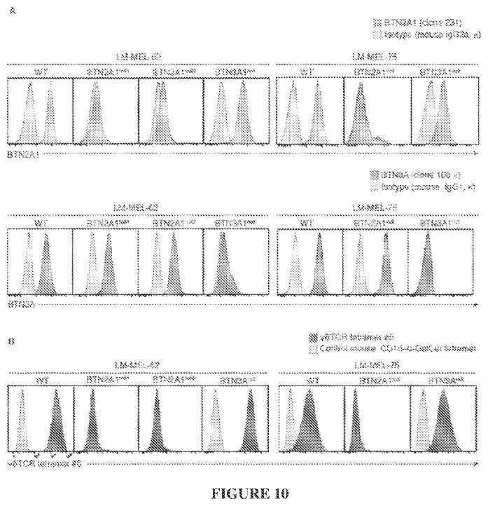

Given that BTN2A1 binds all Vγ9⁺ γδTCRs yet only Vγ9⁺ Vδ2⁺ T cells are pAg-reactive, the inventors hypothesized that Vδ2 is also involved in the interaction. A corollary of this hypothesis could be that separate binding domains on the Vγ9Vδ2⁺ γδTCR, one responsible for binding BTN2A1, located within the germline-encoded region of Vγ9, and another that is also responsible for pAg reactivity, incorporating Vδ2 specificity. Mutations of Vγ9 residues Arg20, Glu70 and His85 (and to a lesser extent Glu22) to Ala all resulted in complete loss of BTN2A1 tetramer reactivity, whereas none of the Vδ2 mutations affected this (FIG. 6A). The side chains of these Vγ9-sensitive residues are in close proximity to one another (Glu70-His85 distance 2.8 Å; His85-Arg20 distance 5.1 Å), and located on the outer faces of the B, D and E strands, respectively, of the ABED antiparallel β-sheet of Vγ9. Together they form a polar triad within the framework region of Vγ9 (FIG. 6B), consistent with BTN2A1 binding to the vast majority of Vγ9⁺ T cells (FIG. 2B). Thus, BTN2A1 appears to bind to the side of Vγ9, distal to the δ-chain and not in the vicinity of the complementarity-determining region (CDR) loops that are typically associated with Ag-recognition.

The inventors next examined which residues were important for mediating functional responses to pAg. Whilst Jurkat cells transduced with γδTCR mutants expressed similar levels of CD3/γδTCR complex on their surface and responded equivalently to immobilized anti-CD3 mAb (FIG. 22), mutations to each of the BTN2A1-binding triad of γ-chain mutants also abrogated pAg-mediated Jurkat cell activation (FIG. 6B). However, mutations to two additional residues, Arg51 of the Vδ2-encoded CDR2 loop, and Lys108 of the CDR3 loop of the TCR γ-chain, also abrogated pAg-mediated activation (FIG. 6C and (H. Wang et al (2010)). These residues had no effect on BTN2A1 binding (FIG. 6B) and were located on the opposite side of the TCR to the putative BTN2A1 footprint (˜30-40 Å separation). However they were in close proximity to one another (˜11 Å)(FIG. 6D), thereby potentially representing a separate binding interface necessary for pAg-mediated activation by the Vγ9Vδ2⁺ γδTCR, but not for BTN2A1 binding. This second binding interface explains the importance of: (i) the Vδ2⁺ TCR δ-chain through involvement of germline-encoded residues, and (ii) the invariant nature of the CDR3γ motif amongst pAg-reactive γδ T cells, via engagement of specific residues within this loop.

Finally, the inventors tested agonist BTN3A1 mAb (clone 20.1)-mediated activation, which is thought to mimic pAg-mediated signaling by conformational modulation or cross-linking of BTN3A1 (C. Harly et al. (2012)). While agonist BTN3A1 mAb-pulsed parental APCs induced Vγ9Vδ2 γδTCR⁺ Jurkat cell activation (FIG. 7), this did not occur with BTN2A1^{null} APCs, suggesting that BTN2A1 is critical for BTN3A1-mediated activation of γδ T cells. Furthermore, Jurkat cells expressing TCR γ-chain Ala mutants of the BTN2A1-binding residues His85, Arg20, and Glu70, as well as BTN2A1-independent mutants of Arg51 (δ-chain) and Lys108 (γ-chain), all failed to respond to parental APCs pulsed with agonist anti-BTN3A1 mAb (FIG. 7). Thus, an interaction between BTN2A1 and the Vγ9⁺ TCR γ-chain is essential, but not sufficient, for BTN3A1-driven γδ T cell responses. This fact may explain why, in earlier studies, the agonist anti-BTN3A1 mAb failed to induce activation of γδ T cells in co-cultures with mouse-derived APCs transfected with human BTN3A1 alone (A. Sandstorm et al. (2014)), because mice do not express BTN2A1.

Accordingly, these mutant studies have revealed the existence of two separate interaction sites on Vγ9Vδ2⁺ γδTCR necessary for pAg- and BTN3A1-mediated activation. One site on the side of the Vγ9 is essential for both BTN2A1 binding and for activation, whereas the other site, incorporating both the Vδ2 CDR2 and γ-chain CDR3 loops, is required for pAg- and BTN3A1-mediated activation. Thus, Vγ9Vδ2⁺ T cells appear to be selectively activated by pAg though a distinct, dual ligand interaction whereby BTN2A1 binds to the Vγ9 domain and another ligand, potentially BTN3A1, binds to a separate interface incorporating both the Vγ9 and Vδ2 domains.

### Concluding Remarks

The findings support a model whereby BTN2A1 and BTN3 associate on the cell surface and are both required for pAg-mediated γδ T cell activation. This model also suggests that after pAg binds BTN3, for example, BTN3A1 via its intracellular B30.2 domain, the BTN2A1-BTN3 complex engages the γδTCR via two distinct binding sites: BTN2A1 binds to Vγ9 framework regions, whereas another ligand, possibly BTN3, for example, BTN3A1, binds to the Vδ2-encoded CDR2 and γ-chain-encoded CDR3 loops on the opposite side of the TCR. This represents a distinct model of Ag-sensing that is highly divergent from canonical MHC-Ag complex recognition by as T cells.

A previous study, using short hairpin RNA (shRNA) knockdown, found no apparent role for BTN2A1 in pAg presentation (S. Vavassori et al. (2013)). However, as the knockdown efficiency was only 81% and BTN2A1 protein was not measured, residual BTN2A1 may have retained functionality. Until now, BTN2A1 has been poorly characterized, with only one earlier study identifying a glycosylation-dependent receptor, CD209 (G. Malcherek et al. (2007)). The inventors found that N-linked glycans were dispensable for BTN2A1 binding to the γδTCR (FIG. 24), making it unlikely that CD209 plays a role in this interaction. Although little is known about the expression pattern of BTN2A1, RNA analysis predicts broad expression on immune cells. The inventors confirmed that BTN2A1 is expressed on circulating T, B, and NK cells, and monocytes, as well as Vγ9Vδ2⁺ T cells (FIG. 24), potentially explaining how γδ T cells can present pAg to themselves (C. T. Morita et al. (1995)).

Recent studies revealed that human BTNL3 and BTNL8 co-associate, and are stimulatory to human Vγ4⁺ γδ T cells, with BTNL3 interacting with a germline-encoded region of the γ-chain variable domain termed the HV4 loop (R. Di Marco Barros et al. (2016); D. Melandri et al. (2018)). Likewise, mouse BTNL1 and BTNL6 are linked and important for intestinal Vγ7⁺ γδ T cell function and appear to bind to a similar region of the γδTCR (R. Di Marco Barros et al. (2016); D. Melandri et al. (2018)). In contrast, the BTN2A1-Vγ9 binding interface appears to exhibit greater dependency on the outer face of the ABED β-sheet of the Vγ9 TCR than the HV4 loop, indicating that the BTN2A1-binding footprint on Vγ9 may be located further away from the CDR loops and closer to the Cy domain. Given the tendency of butyrophilin molecules to dimerize (e.g. BTN3A1 can form stable V-shaped homodimers, and also heterodimers with BTN3A2 (S. Gu et al. (2017)), and BTNL3-BTNL8 heterodimers (D. Melandri et al. (2018)), it is possible that the association between BTN2A1 and BTN3, for example, BTN3A1, represents a direct interaction, although the molecular basis for this remains to be determined.

Compared to other Ag-presenting molecules (MHC and MHC-like molecules), the recognition of heteromeric butyrophilin complexes represents a fundamentally distinct class of immune recognition. It is not yet known how pAg alters this complex in order to induce antigenicity, but it may involve butyrophilin dimer or multimer remodeling, and/or conformational changes to BTN2A1 and BTN3. Other associated molecules such as ABCA1 (B. Castella et al. (2017)) may be directly required.

The findings show that BTN2A1 represents a direct target for agonistic and/or antagonistic intervention in γδ T cell-mediated immunotherapy for infectious disease, cancer, and autoimmunity.

### Tumor Killing/Inhibition Assays

In the following experiments, γδ T cells were enriched by MACS using PE-Cy7-conjugated anti-γδTCR followed by anti-phycoerythrin-mediated magnetic bead purification (Miltenyi Biotec). After enrichment CD3⁺ Vδ2⁺γδ T cells were further purified by sorting using an Aria III (BD). Enriched γδ T cells were stimulated in vitro for 48 h with plate-bound anti-CD3ε (OKT3, μg/ml, Bio-X-Cell), soluble anti-CD28 (CD28.2, 1 μg/ml, BD Pharmingen), phytohemagglutinin (0.5 μg/ml, Sigma), IL-15 (50 ng/ml), and recombinant human IL-2 (100 U/ml, PeproTech), followed by maintenance with IL-2 and IL-15 for 14-21 d. Cells were cultured in complete medium consisting of a 50:50 (v/v) mixture of RPMI-1640 and AIM-V (Invitrogen) supplemented with 10% (v/v) FCS (JRH Biosciences), penicillin (100 U/ml), streptomycin (100 μg/ml), Glutamax (2 mM), sodium pyruvate (1 mM), nonessential amino acids (0.1 mM), and HEPES buffer (15 mM), pH 7.2-7.5 (all from Invitrogen Life Technologies), plus 50 μM 2-mercaptoethanol (Sigma-Aldrich).

LM-MEL-62 and LM-MEL-75 melanoma cells were plated out 1×10⁴ per well in a 96 well flat-bottom plate In RPMI1640 media supplemented with 10% FBS and left to adhere overnight. T25 gamma delta T cells were added at a 2:1 effector:target ratio in TCRPMI with 100U/ml IL-2 and were either stimulated with 5 uM zoledronate, 0.5 ng/ml HMBPP, or left unstimulated. Agonistic antibodies 253, 259 or isotype control BM4 were added to each well at 10 ug/ml. All conditions were repeated in triplicate. Cells were incubated at 37 degrees and on day 3 Vd2+ cells were acquired by flow cytometry. Live cells were gated and activation determined by analysis of CD25 expression. Melanoma cell viability was determined by MTS assay. MTS reagent was added to RPMI media at a 1:5 ratio and 100 ul added per well. Cells were incubated at 37 degrees for 30 minutes and the plate was read on a Spectrostar nano plate reader at 490 nm.

### Measuring γδ T Cell Activation in the Presence of Agonistic Antibodies

In some experiments γδ T cells were enriched by MACS using PE-Cy7-conjugated anti-γδTCR followed by anti-phycoerythrin-mediated magnetic bead purification (Miltenyi Biotec). After enrichment CD3+Vδ2+ γδ T cells were further purified by sorting using an Aria III (BD). Enriched γδ T cells were stimulated in vitro for 48 h with plate-bound anti-CD3ε (OKT3, 10 ug/ml, Bio-X-Cell), soluble anti-CD28 (CD28.2, 1 ug/ml, BD Pharmingen), phytohemagglutinin (0.5 μg/ml, Sigma), IL-15 (50 ng/ml), and recombinant human IL-2 (100 U/ml, PeproTech), followed by maintenance with IL-2 and IL-15 for 14-21 d. Cells were cultured in complete medium consisting of a 50:50 (v/v) mixture of RPMI-1640 and AIM-V (Invitrogen) supplemented with 10% (v/v) FCS (JRH Biosciences), penicillin (100 U/ml), streptomycin (100 μg/ml), Glutamax (2 mM), sodium pyruvate (1 mM), nonessential amino acids (0.1 mM), and HEPES buffer (15 mM), pH 7.2-7.5 (all from Invitrogen Life Technologies), plus 50 μM 2-mercaptoethanol (Sigma-Aldrich).

In vitro pre-expanded CD3+Vδ2+ γδ T cells (5×10⁵) were cultured for 24 hours with HMBPP (0.5 ng/ml) ±10 μg/ml neutralizing anti-BTN2A1 mAb, or isotype control. CD25 expression was determined by flow cytometry, and IFN-γ concentration was determined by cytometric bead array (BD Bioscience) as per manufacturer instructions.

### Identification of BTN2A1 Agonistic Antibodies

The inventors screened the panel of antibodies specific for BTN2A1, as described above, to identify those able to agonise BTN2A1. The inventors assessed the ability of anti-BTN2A1 antibodies to activate γδ T cells. The inventors assessed upregulation of CD25 on the surface of previously expanded γδ T cells following culturing the cells with 10 μg/ml anti-BTN2A1 antibodies or isotype control antibody (BM4) overnight. As shown in FIG. 26A, antibodies 244, 253 and 259 were all able to increase the percentage of γδ T cells expressing CD25.

The inventors additionally measured levels of interferon γ secreted by γδ T cells following culture in the presence of 10 μg/ml anti-BTN2A1 antibodies or isotype control antibody (BM4) overnight. Interferon γ secretion is another indicator of TCR-dependent activation. As shown in FIG. 26B, antibodies 244, 253 and 259 were all able to increase the level of secreted interferon γ.

The inventors additionally tested the ability of anti-BTN2A1 antibodies to activate γδ T cells and kill cancer cells and/or prevent growth of cancer cells in co-culture experiments. Cultures were performed with γδ T cells and melanoma cells (LM-MEL-75 or LM-MEL-62) in a 2:1 ratio. Cells were cultured for three days with antibody 253 or 259 or BM4 (isotype control) or zoledronate (positive control) or HMBPP (positive control). As shown in FIG. 27A, cells cultured in the presence of antibody 253 or 259 induced a similar level of cell lysis of at least one of the melanoma cell lines as the positive controls. FIG. 27B shows activation levels of the γδ T cells as assessed by CD25 upregulation. In particular, FIG. 27B shows the level of expression is upregulated in γδ T cells cultured in the presence of antibody 253 or 259.

### Activation of γδT Cells in the Absence of Phosphoantigen and Induction of Cell Death

### Materials and Methods

### Luminex (PBMCs)

Blood from a healthy donor (Red Cross Australia) was ficolled, PBMC layer collected, washed and 5×10⁵ cells were treated with the indicated antibodies in duplicates @ 10 μg/ml in 500 ul TCRPMI supplemented with 100u/ml IL-2 and incubated at 37 degrees C. and 5% CO₂ for 16 hrs. Supernatants were collected and submitted for Luminex Human 20-plex Inflammation panel analysis (EPX200-12185-901) to Crux Biolabs (Scoresby, VIC, Australia). This measures the following analytes: GM-CSF; ICAM-1, IFN-α; IFN-g, IL-1α, IL-1b, IL-10, IL-12p70, IL-13, IL-17A, IL-4, IL-8, IP-10, MCP-1, IL-6, MIP-1α, MIP-1b, sE-selectin, sP-Selectin, TNF-α. All samples were run with appropriate controls and standards. Treatment with antibody 259 was only performed in one well.

### Luminex (Vγ9Vδ2)

Briefly, Vγ9Vδ2 cells were isolated from 1 healthy donor (Red Cross Australia) and 1 cancer patient derived PBMC using TCRγ/δ+ T Cell Isolation Kit (Miltenyi). Cells were stimulated for 48 hours in TCRPMI supplemented with 100u/ml IL-2, CD3 (10 μg/ml) and CD28 (1 μg/ml). Cells were washed and grown for 14 days in TCRPMI supplemented with 100u/ml IL-2 at 37 degrees C. and 5% CO₂, and then frozen down. The patient provided informed consent and research was approved under HREC 14/425. Vγ9Vδ2 were defrosted and rested overnight in TCRPMI supplemented with 50u/ml IL-2 at 37 degrees C. and 5% CO₂. The next day 2×10⁵ cells were treated with the indicated antibodies in duplicates @ 10 μg/ml or Zoledronic acid (4 μM) or HMBPP (0.5 ng/ml) in 200 ul TCRPMI supplemented with 100u/ml IL-2 and incubated at 37 degrees C. and 5% CO₂ for 16 hrs. Supernatants were collected and submitted for Luminex Human 20-plex Inflammation panel analysis (EPX200-12185-901) to Crux Biolabs (Scoresby, VIC, Australia). All samples were run with appropriate controls and standards.

### In Vitro Killing Assays

Vγ9Vδ2 cells were isolated from either healthy donor (Red Cross Australia) or cancer patient derived PBMC using TCRγ/δ+ T Cell Isolation Kit (Miltenyi). Patients provided informed consent and research was approved under HREC 14/425. Cells were stimulated for 48 hours in TCRPMI supplemented with 100u/ml IL-2, CD3 (10 μg/ml) and CD28 (1 μg/ml). Cells were washed and grown for 14 days in TCRPMI supplemented with 100u/ml IL-2 at 37 degrees C. and 5% CO₂, and then frozen down. Vγ9Vδ2 were defrosted and rested overnight in TCRPMI supplemented with 50u/ml IL-2 at 37 degrees C. and 5% CO₂.

For all assays LM-MEL-62 melanoma cells were plated out at 10,000 cells/well in 100 μl RF10 media in 96-well flat bottomed plates and left to adhere overnight at 37 degrees C. and 5% CO₂.

### E:T Titration

The next day Vγ9Vδ2 cells were washed, counted and added to melanoma cells in TCRPMI supplemented with 100u/ml IL-2 and either 4 uM zoledronate or 10 ug/ml antibody 259, at E:T ratios of 2:1, 1:1, 1:2, 1:4, 1:8 or 1:16.

### Antibody Titration

Vγ9Vδ2 cells were washed, counted and plated out 10,000 cells/well in TCRPMI supplemented with 100u/ml IL-2. Anti-BTN2A1 antibodies 253, 259 or BM4 isotype control were added to wells in duplicate at dilutions of 10, 1, 0.1 and 0.01 ug/ml. Cells were incubated at 37 degrees C. and 5% CO₂. At day 3 Vγ9Vδ2 cells were washed out and 100 μl MTS reagent was added to wells for 1 hour as per manufacturers protocol (Promega, USA). Plates were subsequently read at 490 nm using a Spectrostar Nano microplate reader (BMG Labtech) to determine cell viability corrected for background absorbance.

### Flow-Cytometry

Vγ9Vδ2 cells were stained for CD3, Vδ2, CD25 and live dead viability dye as previously described and analysed on a Canto flow cytometer (BD). Cells were gated on lymphocytes, single cells, live cells, CD3+ Vδ2+, and activation determined based on CD25 expression.

### Discussion

Vγ9Vδ2 cells react on phosphoantigen presentation with upregulation of activation markers including CD25 and CD69 as well as cytokine expression. This requires BTN2A1 and BTN3A1 expression on the surface of the antigen-presenting cell. Anti-BTN2A1 antibodies 259 and 253 can mimic phosphoantigen mediated activation to various degrees without the presence of these intermediaries of the melavonate/non-mevalonate pathway (FIG. 28A).

Functionally, this activation leads to a dose-dependent increase in the ability of pre-expanded Vγ9Vδ2T cells to recognize and kill melanoma tumour cells (herein referred to as: LM-MEL-62). Vγ9Vδ2 cells derived from 2 different donors (melanoma patients) were pre-expanded and co-incubated with melanoma cells (1:1 ratio) and treated with different amounts of antibody 253 or antibody 259. Analogous to the activation data in FIG. 28A, treatment with antibody 259 led to lower viability rates of LM-MEL-62 cells when compared to antibody 253. Higher concentrations of antibody 259 enhanced Vγ9Vδ2 Cell mediated tumour cell killing with maximum killing across both donors achieved between 1 and 10 μg/ml (FIG. 28B).

Tumour cell killing was not only dependent on the dose of antibody, but on the ratio of effector (Vγ9Vδ2) to target (LM-MEL-62) cells (FIG. 28C). When compared to treatment with zoledronic acid, antibody 259 mediated cell killing showed correlation to E:T (more effectors led to higher killing), albeit killing occurred to a lesser extent across both donors. Interestingly, Vγ9Vδ2 cells derived from a cancer patient (patient 1) seemed to be less capable in tumour cell killing independent of the used stimulus than healthy donor derived ones. This suggests a (reversible) functional alteration of Vγ9Vδ2 cells in the setting of cancer, potentially extending beyond the tumour microenvironment (the cells were isolated from circulation).

These differences were reflected partially in cytokine/chemokine profiles derived from in-vitro expanded Vγ9Vδ2 cells and activated with Zoledronate, HMBPP or treated with different antibodies as indicated (FIGS. 29 A and B, Tables 3 and 4). In healthy donors and patient derived Vγ9Vδ2, treatment with Zoldronate and HMBPP led to an increase in expression/secretion of GMCSF, ICAM-1, IFNγ, IL-13, MIP-1a, MIP-1b, sE-selectin, sP-Selectin and TNF. With the exception of ICAM1 all of these were higher expressed when the stronger stimulus—HMBPP—was used, ICAM-1 expression was upregulated to a similar extent with zoledronate and HMBPP. Secretion of IL-17A and IL-4 could only be detected in the setting of HMBPP activation. Given the immune-suppressive function of IL-17, this demonstrates the necessity to being able to fine-tune activation and blocking signal to achieve the most desired outcome. Treatment with the 2 agonistic anti-BTN2A1 antibodies 253 and 259 showed a similar pattern to treatment with Zoledronate and HMBPP, however 253 was a weaker stimulus.

TABLE 3
Cytokine and chemokine expression in γδT cells from patient 1 as shown in FIG. 29A (in pg/ml)

| | No stim | | Zoledronate | | HMBPP | | BM4 (isotype) | | Hu34C1 | | 253 | | 259 | |
|---|---|---|---|---|---|---|---|---|---|---|---|---|---|---|
| GMCSF | 47.16 | 38.57 | 650.17 | 619.93 | 2172.54 | 2625.54 | 38.35 | 41.74 | 9.22 | 8.09 | 32.02 | 33.23 | 1135.87 | 1151.41 |
| ICAM-1 | 713.92 | 484.56 | 968.87 | 888.51 | 729.5 | 885.22 | 617.01 | 797.95 | 0.1 | 0.1 | 480.81 | 405.75 | 798.07 | 755.13 |
| IFNγ | 7.62 | 8.02 | 175.86 | 171.42 | 723.16 | 791.56 | 5.81 | 9.32 | 0.1 | 0.1 | 6.87 | 3.45 | 598.1 | 740.33 |
| IL-13 | 55.34 | 48.28 | 235.43 | 219.81 | 478.3 | 602.86 | 48.5 | 62.16 | 14.86 | 16.14 | 45.16 | 36.92 | 399.99 | 461.27 |
| IL-17A | 0.27 | 0.1 | 3.5 | 4.08 | 9.23 | 10.85 | 0.01 | 1.14 | 0.1 | 0.1 | 0.1 | 0.1 | 5.86 | 6.55 |
| IL-4 | 0.1 | 0.1 | 7.43 | 7.68 | 875.51 | 1027.45 | 0.1 | 0.1 | 0.1 | 0.1 | 0.1 | 0.1 | 490.79 | 389.47 |
| IL-8 | 0.1 | 0.1 | 0.1 | 0.1 | 2.27 | 5.47 | 0.1 | 0.1 | 0.1 | 0.1 | 0.1 | 0.1 | 0.93 | 1.06 |
| MIP-1a | 158.96 | 143.21 | 274.25 | 273.85 | 321.54 | 383.73 | 157.35 | 165.66 | 51.38 | 50.75 | 133.36 | 137.25 | 317.76 | 361.32 |
| MIP-1b | 1134.97 | 977.97 | 5061.49 | 4756.68 | 9453.77 | 12721.52 | 1011.59 | 1046.83 | 252.05 | 239.49 | 901.33 | 941.65 | 6921.27 | 8238.42 |
| sE-selectin | 210.49 | 196.91 | 259.01 | 287.88 | 373.32 | 372.16 | 179.13 | 201.21 | 0.1 | 78.08 | 142.97 | 101.78 | 311.65 | 368.09 |
| sP-Selectin | 0.1 | 0.1 | 1239.34 | 1299.54 | 2542.23 | 2775.99 | 0.1 | 0.1 | 0.1 | 0.1 | 0.1 | 0.1 | 2170.46 | 2424.08 |
| TNFα | 0.1 | 0.1 | 105.84 | 102.44 | 2973.1 | 3657.48 | 0.1 | 0.1 | 0.1 | 0.1 | 0.1 | 0.1 | 1130.35 | 1137.89 |

TABLE 4
Cytokine and chemokine expression in γδT cells from a healthy donor as shown in FIG. 29B (in pg/ml)

| | No stim | | Zoledronate | | HMBPP | | BM4 (isotype) | | Hu34C1 | | 253 | | 259 | |
|---|---|---|---|---|---|---|---|---|---|---|---|---|---|---|
| GMCSF | 4.55 | 8.28 | 254.29 | 268.62 | 2375.52 | 2349.07 | 8.18 | 5.87 | 7.12 | 3.08 | 7.52 | 16.6 | 578.23 | 608.66 |
| ICAM-1 | 0.1 | 0.1 | 490.35 | 504.11 | 575.02 | 570.14 | 0.1 | 0.1 | 0.1 | 0.1 | 0.1 | 0.1 | 227.66 | 226.25 |
| IFNγ | 0.1 | 0.1 | 58.38 | 58.97 | 751.87 | 798.38 | 0.1 | 0.1 | 0.1 | 0.1 | 0.1 | 0.1 | 359.53 | 412.2 |
| IL-13 | 0.1 | 0.1 | 5.22 | 4.43 | 84.37 | 87.99 | 0.1 | 0.1 | 0.1 | 0.1 | 0.1 | 0.1 | 23.33 | 31.31 |
| IL-17A | 0.1 | 0.1 | 0.1 | 0.1 | 8.39 | 9.97 | 0.1 | 0.1 | 0.1 | 0.1 | 0.1 | 0.1 | 3.7 | 4.17 |
| IL-4 | 0.1 | 0.1 | 0.1 | 0.1 | 137.56 | 117.91 | 0.1 | 0.1 | 0.1 | 0.1 | 0.1 | 0.1 | 0.1 | 0.1 |
| IL-8 | 0.1 | 0.1 | 0.1 | 0.1 | 0.1 | 0.1 | 0.1 | 0.1 | 0.1 | 0.1 | 0.1 | 0.1 | 0.1 | 0.1 |
| MIP-1a | 13.33 | 15.24 | 121.61 | 125.27 | 395.47 | 392.74 | 16.25 | 13.9 | 7.83 | 9.16 | 16.32 | 19.57 | 301.77 | 286.07 |
| MIP-1b | 99.18 | 108.1 | 1280.91 | 1334.19 | 19239.36 | 20123.08 | 114.05 | 97.92 | 43.18 | 50.62 | 117.31 | 143.67 | 9757.6 | 9064.66 |
| sE-selectin | 0.1 | 0.1 | 190.9 | 190.9 | 387.87 | 373.47 | 0.1 | 0.1 | 0.1 | 0.1 | 0.1 | 0.1 | 259.93 | 300.24 |
| sP-Selectin | 0.1 | 0.1 | 200.54 | 313.85 | 3183.34 | 3082.95 | 0.1 | 0.1 | 0.1 | 0.1 | 0.1 | 0.1 | 2291.35 | 2223.07 |
| TNFα | 0.1 | 0.1 | 13.03 | 13.02 | 2186.67 | 2148.53 | 0.1 | 0.1 | 0.1 | 0.1 | 0.1 | 0.1 | 451.81 | 481.49 |

In healthy donor derived Vγ9Vδ2 cells, treatment with antibody 253 led to only small increases in the amounts of secreted GMCSF, but in no other measured cytokine/chemokine. No increase in any analyte was detected in cancer patient derived Vγ9Vδ2 cells, however basal levels of multiple analytes were higher than in healthy donor derived cells which often had no detectable expression when treated with isotype antibody alone (BM4).

Antibody 259 led to substantial increase in multiple analytes across both donors, including GMCSF, IFNγ, IL-13, IL-17 (very low), MIP1α and MIP1β, sE- and sP-selectins, and TNF. Unique to healthy donor derived cells was an increase in ICAM1 upon antibody 259 treatment and in cancer derived cells a de novo expression of IL-4 to more than 400 μg/ml. In healthy donors, no IL-4 was detected upon antibody 259 treatment.

The inventors additionally used a BTN2A1 antagonistic antibody to explore which baseline expressed cytokines within the pre-expanded Vγ9Vδ2 cells can be blocked by inhibiting BTN2A1 gamma-delta TCR binding/activation. In healthy donor cells 34C1 treatment led to downregulation of MIP1α and b as well as GMCSF when compared to isotype (BM4) treated controls. In cancer patient derived cells with much higher baseline expression of cytokines reduction in levels of GMCSF, ICAM1 (to undetectable levels), IFNγ, IL-13, MIP1α and MIP1β as well as sE-selectin could be detected, and BTN2A1 blockade may be a valid treatment strategy to reduce these factors.

To explore how our agonistic anti-BTN2A1 antibody 259 influences cytokine/chemokine expression in the context of a PBMC, the inventors treated freshly isolated PBMCs from a healthy donor with antibody 259 and antibody 229 (antagonistic anti-BTN2A1 antibody) as well as isotype control. The consequences for cytokine expression of inhibitory signals in a baseline setting for BTN2A1 blockade and activating signals (259) were explored, including signals from other immune cell subsets expressing BTN2A1 and/or 3A1, or secondary effects of Vγ9Vδ2 cell activation.

In line with earlier data derived from isolated Vγ9Vδ2 cell cultures, antibody 259 increased expression of IFNγ and sE-Selectin in the context of a full PBMC, and both the BTN2A1 and the BTN3A1 inhibitory antibodies blocked baseline expression (FIG. 30). Other signals present in the highly enriched Vγ9Vδ2 cell cultures were not detected in the context of a full PBMC. Most prominently, no increase in TNFα levels upon contact with antibody 259 were detected (Table 5). This may be due to the small number of Vγ9Vδ2 cells within a PBMC, diluting the signal beyond the detection threshold.

TABLE 5
Cytokine and chemokine expression in PBMCs
as shown in FIG. 30 (in pg/ml)

| | BM4 (isotype) | | 229 | | 259 |
|---|---|---|---|---|---|
| ICAM-1 | 1451.68 | 1567.34 | 927.93 | 797.62 | 1578.36 |
| IFNγ | 11.68 | 13.25 | 5.41 | 1.9 | 104.96 |
| IL-1α | 3.02 | 5.74 | 2.32 | 2.24 | 8.7 |
| IL-1β | 1.38 | 1.47 | 0.87 | 0.75 | 5.19 |
| IL-10 | 37.79 | 41.64 | 24.72 | 24.98 | 49.99 |
| IL-17A | 14.73 | 17.44 | 11.7 | 10.19 | 24.59 |
| IL-8 | 137.16 | 330.06 | 106.14 | 100.49 | 495.89 |
| IP-10 | 666.12 | 579.47 | 540.03 | 447.54 | 641.08 |
| MCP-1 | 613.84 | 630.52 | 265.72 | 248.77 | 1023.7 |
| IL-6 | 0 | 60.03 | 0 | 0 | 462.59 |
| MIP-1α | 139.97 | 147.87 | 64.48 | 62.52 | 131.71 |
| MIP-1β | 715.37 | 719.75 | 415.15 | 364.18 | 584.34 |
| sE-Selectin | 280.91 | 291.08 | 188.41 | 208.68 | 327.64 |

Interestingly, the inventors saw additional cytokine/chemokines being up-regulated by 259 which were not detected in the mono-cultures. These included IL-8 (CXCL8) another chemoattractant for immune cells, mainly neutrophils and T cells (Henkels et al 2011) which may play a prominent role in autoimmune conditions like psoriasis by attracting T cells (Zheng et al 1998). Additionally, the inventors saw increase in CCL2 (MCP-1), a strong chemoattractant of dendritic cells and IL-6 as prototypical and key interleukin associated with inflammatory processes upon 259 treatment (Erlandsson et al, 2017; Hashizume et al., 2015). All of these cytokines/chemokines were downregulated by blockade of the BTN2A1/3A1 signalling axis with 229, confirming their reliance of signalling via this complex.

Some cytokines and chemokines were reduced below isotype control levels with the antagonistic antibodies, even though their expression was not enhanced by treatment with antibody 259. These included ICAM-1, MIP1α and MIP1β and sE-Selectin.

### Activation of Vδ2− γδ T Cells

### Methods

Mouse 3T3 fibroblast cells were transfected with full-length human CD1c or CD1d heavy chains, or a control construct (BTNL3) using a pMSCV-IRES-GFP plasmid and Fugene transfection reagent. After ˜2 d when the 3T3 cells expressed surface CD1c or CD1d, they were co-cultured with human T cell lines that expressed human γδTCRs specific for either CD1c (Vγ9Vδ1+) or CD1d (Vγ9Vδ1+, or Vγ5Vδ1+) for 24 h, after which the level of activation on the T cell lines was determined by flow cytometry using CD69. T cell lines were cultured on immobilised anti-CD3/anti-CD28 as a positive control, or cultured with untransfected 3T3 cells as a negative control.

### Discussion

To test the ability of BTN2A1 to augment Vδ2− γδ T cell reactivity to their cognate ligands, the inventors performed in vitro assays using two γδ T cell lines, both Vγ9Vδ1 γδTCR+, that are reactive to CD1c and CD1d, respectively (FIG. 31). The inventors also included a control Vγ5Vδ1+ γδ T cell line (clone 9C2; A. P. Uldrich et al. (2013)) that is also CD1d-reactive but should not bind BTN2A1 because it lacks Vγ9. The inventors transfected mouse 3T3 APCs with either human CD1c or CD1d, plus BTN2A1 or an irrelevant control construct (human BTNL3) and co-cultured them with the γδ T cell lines and measured activation (CD69) after 24 h. The data show that BTN2A1 can (a) induce some activation of these Vγ9+ γδ T cell lines even in the absence of additional TCR ligands, and (b) augment the activation of both CD1c- and CD1d-specific γδTCRs. This appeared to be specific to Vγ9+ TCRs since whilst 9C2 (Vγ5+) reacted specifically to CD1d, this was not enhanced by BTN2A1 expression.

These findings indicate that in addition to being essential for the activation of Vγ9Vδ2+ γδ T cells, BTN2A1 can also directly induce the activation of Vδ2− γδ T cells, and can also augment the responses of these cells to their cognate Ag.

### REFERENCES

Al-Lazikani et al., (1997) Journal of molecular biology, 273:927-948
Ausubel et al., (1987) Current Protocols in Molecular Biology Wiley, Boston
Ausubel et al., (1988, including all updates until present) Current Protocols in Molecular Biology,
Greene Pub. Associates and Wiley-Interscience
Bork et al., (1994) Journal of molecular biology, 242:309-320
Brown, (1991) Essential Molecular Biology: A Practical Approach, Vol. 1 and 2, IRL Press
Chothia and Lesk, (1987) Journal of molecular biology, 196:901-917
Chothia et al., (1989) Nature, 342:877-883
Coligan et al., (all updates until present) Current Protocols in Immunology, John Wiley & Sons
Edelman et al., (1969) PNAS, 63:78-85,
Erlandsson, et al., (2017) Biochim Biophys Acta Mol Basis Dis. 1863(9): 2158-2170.
Glover and Hames, (1995 and 1996) DNA Cloning: A Practical Approach, Volumes 1-4, IRL Press
Harlow and Lane, (1988) Antibodies: A Laboratory Manual, Cold Spring Harbor Laboratory
Hashizume, and Ohsugi, (2015) Endocr Metab Immune Disord Drug Targets.
Henkels et al., (2011) FEBS Lett. 585(1):159-66
Holliger et al., (1993) PNAS, 90:6444-6448
Jones et al., (2010) Journal of immunological methods, 354:85-90
Jostock et al., (2004) Journal of immunological methods, 289:65-80
Kabat, (1987 and 1991) Sequences of Proteins of Immunological Interest, National Institutes of
Health, Bethesda, Md.
Keler et al., (1997) Cancer Research, 57:4008-4014
Kim et al., (2015) PloS one, 10(3):e0121171
Kostelny et al., (1992) The Journal of Immunology, 148:1547-1553
Largaespada et al., (1996) Journal of immunological methods 197:85-95,
Merchant et al., (1998) Nature Biotechnology, 16:677-681
Milstein et al., (1983) Nature, 305:537-540
Novotny et al., (1991) PNAS, 88:8646-8650
Osol, (1980) Remington's Pharmaceutical Science, 16th Edition, Mack Publishing Company
Remington's Pharmaceutical Sciences (Mack Publishing Co. N.J. USA, 1991)
Palakodeti et al., (2012) J. Biological Chemistry, 287(39):32780-90
Perbal, (1984) A Practical Guide to Molecular Cloning, John Wiley and Sons
Ridgway et al., (1996) Protein Engineering, 9:617-621
Sambrook and Russell, (2001) Molecular Cloning: A Laboratory Manual, 3^{rd} Edition, Cold Spring Harbor Laboratory Press
Sambrook et al., (1989) Molecular Cloning: A Laboratory Manual, Cold Spring Harbor Laboratory Press
Schutters et al., (2013) Cell Death and Differentiation, 20:49-56,
Scopes, (1994) Protein purification: principles and practice, 3^{rd} Edition, Springer Verlag
Thapa et al., (2008) Journal of cellular and molecular medicine, 12:1649-1660
Ungethüm et al., (2011) The Journal of Biological Chemistry, 286:1903-1910
Zheng, et al. (1998) Chin Med J (Engl). 111(2): p. 166-8
J. Rossjohn et al., T cell antigen receptor recognition of antigen-presenting molecules. Annu Rev Immunol 33, 169-200 (2015)
P. Constant et al., Stimulation of human gamma delta T cells by nonpeptidic mycobacterial ligands. Science 264, 267-270 (1994).
Y. Tanaka et al., Natural and synthetic non-peptide antigens recognized by human gamma delta T cells. Nature 375, 155-158 (1995).
L. Zhao, W. C. Chang, Y. Xiao, H. W. Liu, P. Liu, Methylerythritol phosphate pathway of isoprenoid biosynthesis. Annu Rev Biochem 82, 497-530 (2013).
A. Sandstrom et al., The intracellular B30.2 domain of butyrophilin 3A1 binds phosphoantigens to mediate activation of human Vgamma9Vdelta2 T cells. Immunity 40, 490-500 (2014).
Y. L. Wu et al., gammadelta T cells and their potential for immunotherapy. Int J Biol Sci 10, 119-135 (2014).
J. Zheng, Y. Liu, Y. L. Lau, W. Tu, gammadelta-T cells: an unpolished sword in human anti-infection immunity. Cellular & molecular immunology 10, 50-57 (2013).
L. Wang, A. Kamath, H. Das, L. Li, J. F. Bukowski, Antibacterial effect of human V gamma 2V delta 2 T cells in vivo. The Journal of clinical investigation 108, 1349-1357 (2001).
D. I. Godfrey, J. Le Nours, D. M. Andrews, A. P. Uldrich, J. Rossjohn, Unconventional T Cell Targets for Cancer Immunotherapy. Immunity 48, 453-473 (2018).
H. Wang, Z. Fang, C. T. Morita, Vgamma2Vdelta2 T Cell Receptor recognition of prenyl pyrophosphates is dependent on all CDRs. Journal of immunology 184, 6209-6222 (2010).
C. T. Morita et al., Direct presentation of nonpeptide prenyl pyrophosphate antigens to human gamma delta T cells. Immunity 3, 495-507 (1995).
C. Harly et al., Key implication of CD277/butyrophilin-3 (BTN3A) in cellular stress sensing by a major human gammadelta T-cell subset. Blood 120, 2269-2279 (2012).
D. A. Rhodes et al., Activation of human gammadelta T cells by cytosolic interactions of BTN3A1 with soluble phosphoantigens and the cytoskeletal adaptor periplakin. Journal of immunology 194, 2390-2398 (2015).
Z. Sebestyen et al., RhoB Mediates Phosphoantigen Recognition by Vgamma9Vdelta2 T Cell Receptor. Cell Rep 15, 1973-1985 (2016).
S. Gu et al., Phosphoantigen-induced conformational change of butyrophilin 3A1 (BTN3A1) and its implication on Vgamma9Vdelta2 T cell activation. Proceedings of the National Academy of Sciences of the United States of America 114, E7311-E7320 (2017).
M. Salim et al., BTN3A1 Discriminates gammadelta T Cell Phosphoantigens from Nonantigenic Small Molecules via a Conformational Sensor in Its B30.2 Domain. ACS Chem Biol 12, 2631-2643 (2017).
Y. Yang et al., A Structural Change in Butyrophilin upon Phosphoantigen Binding Underlies Phosphoantigen-Mediated Vgamma9Vdelta2 T Cell Activation. Immunity 50, 1043-1053 e1045 (2019).
L. Starick et al., Butyrophilin 3A (BTN3A, CD277)-specific antibody 20.1 differentially activates Vgamma9Vdelta2 TCR clonotypes and interferes with phosphoantigen activation. European journal of immunology 47, 982-992 (2017).
F. Riano et al., Vgamma9Vdelta2 TCR-activation by phosphorylated antigens requires butyrophilin 3 A1 (BTN3A1) and additional genes on human chromosome 6. European journal of immunology 44, 2571-2576 (2014).
J. Young et al., Genome-scale CRISPR-Cas9 knockout and transcriptional activation screening. Nature protocols 12, 828-863 (2017).
G. Malcherek et al., The B7 homolog butyrophilin BTN2A1 is a novel ligand for DC-SIGN. Journal of immunology 179, 3804-3811 (2007).
P. Batard et al., Use of phycoerythrin and allophycocyanin for fluorescence resonance energy transfer analyzed by flow cytometry: advantages and limitations. Cytometry 48, 97-105 (2002).
T. J. Allison, C. C. Winter, J. J. Fournie, M. Bonneville, D. N. Garboczi, Structure of a human gammadelta T-cell antigen receptor. Nature 411, 820-824 (2001).
A. P. Uldrich et al., CD1d-lipid antigen recognition by the gammadelta TCR. Nature immunology 14, 1137-1145 (2013).
P. Vantourout et al., Heteromeric interactions regulate butyrophilin (BTN) and BTN-like molecules governing gammadelta T cell biology. Proceedings of the National Academy of Sciences of the United States of America 115, 1039-1044 (2018).
S. Vavassori et al., Butyrophilin 3A1 binds phosphorylated antigens and stimulates human gammadelta T cells. Nature immunology 14, 908-916 (2013).
R. Di Marco Barros et al., Epithelia Use Butyrophilin-like Molecules to Shape Organ-Specific gammadelta T Cell Compartments. Cell 167, 203-218 e217 (2016).
D. Melandri et al., The gammadeltaTCR combines innate immunity with adaptive immunity by utilizing spatially distinct regions for agonist selection and antigen responsiveness. Nature immunology 19, 1352-1365 (2018).
B. Castella et al., The ATP-binding cassette transporter A1 regulates phosphoantigen release and Vgamma9Vdelta2 T cell activation by dendritic cells. Nature communications 8, 15663 (2017).
S. Chen et al., Genome-wide CRISPR screen in a mouse model of tumor growth and metastasis. Cell 160, 1246-1260 (2015).
M. Martin, Cutadapt removes adapter sequences from high-throughput sequencing reads. 2011 17, 3% J EMBnet.journal (2011).
M. D. Robinson, D. J. McCarthy, G. K. Smyth, edgeR: a Bioconductor package for differential expression analysis of digital gene expression data. Bioinformatics 26, 139-140 (2010).
A. R. Aricescu, W. Lu, E. Y. Jones, A time- and cost-efficient system for high-level protein production in mammalian cells. Acta Crystallogr D Biol Crystallogr 62, 1243-1250 (2006

1. A method for inhibiting activation of γδ T cells that express a Vγ9+ TCR in a subject, the method comprising administering a BTN2A1 antagonist to the subject, wherein the BTN2A1 antagonist:
i) inhibits formation of a BTN2A1/BTN3A1 complex on the surface of a cell;
ii) inhibits binding of BTN2A1 to Vγ9;
iii) inhibits binding of a BTN2A1/BTN3A1 complex to the Vγ9+ TCR; and/or
iv) decreases the activity and/or survival of cells that express BTN2A1.

2. The method of claim 1, wherein the method inhibits activation of Vγ9Vδ2+ γδ T cells or inhibits activation of Vγ9Vδ2− γδ T cells.

3. (canceled)

4. The method of claim 1, wherein the BTN2A1/BTN3A1 complex comprises one or more additional molecules and/or the BTN2A1/BTN3A1 complex comprises BTN3A2 and/or BTN3A3.

5. (canceled)

6. The method of claim 1, wherein:
(i) the method inhibits one or more of cytolytic function, cytokine production of one or more cytokines, or proliferation of the γδ T cells; and/or
(ii) the BTN2A1 antagonist inhibits phosphoantigen mediated activation of the γδ T cells
(iii) the BTN2A1 antagonist inhibits association of BTN2A1 and BTN3A1; and/or
(iv) the BTN2A1 antagonist inhibits direct association of BTN2A1 and BTN3A1; and/or
(v) the BTN2A1 antagonist inhibits binding of BTN2A1 to the germline-encoded region of Vγ9 and/or distal to the δ-chain; and/or
(vi) the BTN2A1 antagonist modifies one or more of the extracellular domains (IgV and/or IgC) of the BTN2A1 molecule to switch the BTN2A1 molecule from stimulatory BTN2A1 to that of non-stimulatory.

7. (canceled)

8. (canceled)

9. (canceled)

10. (canceled)

11. The method of claim 2, wherein the BTN2A1 antagonist inhibits binding of a BTN2A1/BTN3A1 complex to the germline-encoded regions of Vδ2 such as the CDR2 loop and/or the CDR3 loop of the TCR 7 chain.

12. (canceled)

13. The method of claim 6, wherein the BTN2A1 antagonist modifies one or more of the extracellular domains (IgV and/or IgC) of the BTN2A1 molecule and inhibits phosphoantigen activation.

14. A method of suppressing or inhibiting Vγ9+ γδ T cell responses in a subject, wherein the method comprises administering a BTN2A1 antagonist to the subject, wherein the BTN2A1 antagonist:
i) inhibits formation of a BTN2A1/BTN3A1 complex on the surface of a cell;
ii) inhibits binding of BTN2A1 to Vγ9+ TCR;
iii) inhibits binding of a BTN2A1/BTN3A1 complex to the Vγ9+ TCR; and/or
iv) decreases the activity and/or survival of cells that express BTN2A1.

15. The method of claim 14, wherein the method suppresses or inhibits Vγ9Vδ2+ γδ T cell responses.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. A method for inhibiting activation of γδ T cells that express a Vγ9+ TCR in vitro or ex vivo, the method comprising culturing the γδ T cells and cells expressing BTN2A1 in the presence of a BTN2A1 antagonist, wherein the BTN2A1 antagonist inhibits:
i) formation of a BTN2A1/BTN3A1 complex on the surface of the cells;
ii) binding of BTN2A1 to Vγ9; and/or
iii) binding of a BTN2A1/BTN3A1 complex to the Vγ9+ TCR.

29. (canceled)

30. The method of claim 28, wherein the method further comprises the step of administering the γδ T cells to a subject in need thereof.

31. (canceled)

32. (canceled)

33. A method for activating γδ T cells that express a Vγ9+ TCR in a subject, the method comprising administering a BTN2A1 agonist to the subject, wherein the BTN2A1 agonist:
i) promotes formation of a BTN2A1/BTN3A1 complex on the surface of a cell;
ii) induces ligation of Vγ9+ TCR on γδ T cells; and/or
iii) increases the activity and/or survival of cells that express BTN2A1.

34. The method of claim 33, wherein the method activates Vγ9Vδ2+ γδ T cells and/or the method activates Vγ9Vδ2− γδ T cells.

35. (canceled)

36. The method of claim 33, wherein the BTN2A1/BTN3A1 complex comprises one or more additional molecules and/or the BTN2A1/BTN3A1 complex comprises BTN3A2 and/or BTN3A3.

37. (canceled)

38. The method of claim 33, wherein:
(i) the method activates one or more of cytolytic function, cytokine production, or proliferation of the γδ T cells; and/or
(ii) the BTN2A1 agonist activates the γδ T cells independent of phosphoantigen binding; and/or
(iii) the BTN2A1 agonist promotes association of BTN2A1 and BTN3A1; and/or
(iv) the BTN2A1 agonist promotes direct association of BTN2A1 and BTN3A1; and/or
(v) the BTN2A1 agonist is bi-specific for BTN2A1 and BTN3A1; and/or
(vi) the BTN2A1 agonist cross-reacts with BTN3A1; and/or
(vii) the BTN2A1 agonist modifies one or more of extracellular domains (IgV and/or IgC) of the BTN2A1 molecule to switch the BTN2A1 molecule from non-stimulatory BTN2A1 to that of stimulatory.

39. (canceled)

40. (canceled)

41. (canceled)

42. (canceled)

43. (canceled)

44. (canceled)

45. A method of inducing or enhancing Vγ9+ γδ T cell responses in a subject, wherein the method comprises administering a BTN2A1 agonist to the subject, wherein the BTN2A1 agonist:
i) promotes formation of a BTN2A1/BTN3A1 complex on the surface of a cell;
ii) induces ligation of Vγ9+ TCR on γδ T cells; and/or
iii) increases the activity and/or survival of cells that express BTN2A1.

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. A method for activating γδ T cells that express a Vγ9+ TCR in vitro or ex vivo, the method comprising culturing the γδ T cells and cells expressing BTN2A1 in the presence of a BTN2A1 agonist, wherein the BTN2A1 agonist:
i) promotes formation of a BTN2A1/BTN3A1 complex on the surface of antigen presenting cells;
ii) induces ligation of Vγ9+ TCR on γδ T cells; and/or
iii) increases the activity and/or survival of cells that express BTN2A1.

58. (canceled)

59. The method of claim 57, wherein the method further comprises the step of administering the activated γδ T cells to a subject in need thereof.

60. (canceled)

61. (canceled)

62. A BTN2A1 antagonist, wherein the BTN2A1 antagonist specifically binds to BTN2A1 and inhibits:
i) formation of a BTN2A1/BTN3A1 complex on the surface of a cell;
ii) binding of BTN2A1 to Vγ9; and/or
iii) binding of a BTN2A1/BTN3A1 complex to the Vγ9+ TCR.

63. (canceled)

64. A BTN2A1 agonist, wherein the BTN2A1 agonist specifically binds to BTN2A1 and:
i) promotes formation of a BTN2A1/BTN3A1 complex on the surface of a cell;
ii) induces ligation of Vγ9+ TCR on γδ T cells; and/or
iii) increases the activity and/or survival of cells that express BTN2A1.

65. (canceled)

66. The method of claim 1, wherein BTN2A1 antagonist is a protein comprising an antigen binding domain, wherein the protein is:
(i) a single chain Fv fragment (scFv);
(ii) a dimeric scFv;
(iii) a Fv fragment;
(iv) a single domain antibody (sdAb);
(v) a nanobody;
(vi) a diabody, triabody, tetrabody or higher order multimer;
(vii) Fab fragment;
(viii) a Fab′ fragment;
(ix) a F(ab′) fragment;
(x) a F(ab′)₂ fragment;
(xi) any one of (i)-(x) linked to a Fc region of an antibody;
(xii) any one of (i)-(x) fused to an antibody or antigen binding fragment thereof that binds to an immune effector cell; or
(xiii) an antibody.

67. (canceled)

68. (canceled)

69. The method of claim 1, wherein the BTN2A1 antagonist is a soluble Vγ9+ TCR.

70. The method of claim 69, wherein the soluble Vγ9+ TCR is a monomer or the soluble Vγ9+ TCR is a multimer.

71. (canceled)

72. The method of claim 1, wherein the BTN2A1 antagonist is:
(i) an antibody comprising a heavy chain variable region (V_{H}) comprising a sequence set forth in SEQ ID NO: 100 and a light chain variable region (V_{L}) comprising a sequence set forth in SEQ ID NO: 101; or
(ii) an antibody comprising a heavy chain variable region (V_{H}) comprising a sequence set forth in SEQ ID NO: 108 and a light chain variable region (V_{L}) comprising a sequence set forth in SEQ ID NO: 109; or
(iii) an antibody comprising a heavy chain variable region (V_{H}) comprising a sequence set forth in SEQ ID NO: 116 and a light chain variable region (V_{L}) comprising a sequence set forth in SEQ ID NO: 117; or
(iv) an antibody comprising a heavy chain variable region (V_{H}) comprising a sequence set forth in SEQ ID NO: 124 and a light chain variable region (V_{L}) comprising a sequence set forth in SEQ ID NO: 125; or
(v) an antibody comprising a heavy chain variable region (V_{H}) comprising a sequence set forth in SEQ ID NO: 132 and a light chain variable region (V_{L}) comprising a sequence set forth in SEQ ID NO: 133.

73. (canceled)

74. (canceled)

75. (canceled)

76. (canceled)

77. A BTN2A1 agonist that specifically binds to BTN2A1 and:
(i) activates γδ T cells and/or increases the number of activated γδ T cells in a population of cells; and/or
(i) increases the percentage of γδ T cells expressing a marker of γδ T cell activation; and/or
(ii) increases secretion of a cytokine by γδ T cells; and/or
(iii) induces γδ T cells to kill cancer cells and/or inhibit growth of the cancer cells and/or kill infected cells and/or inhibit growth of infected cells; and/or
(iv) increases the amount of a marker of γδ T cell activation expressed on the cell surface of γδ T cells.

78. A BTN2A1 agonist that specifically binds to BTN2A1 and:
(i) increases the percentage of γδ T cells expressing CD25 on the cell surface; and/or
(ii) increases secretion of interferon 7 by γδ T cells; and/or
(iii) induces γδ T cells to kill cancer cells and/or inhibit growth of the cancer cells; and/or
(iv) increases the amount of CD25 expressed on the cell surface of γδ T cells.

79. The BTN2A1 agonist of claim 77, which is an antibody comprising:
(i) a light chain variable region (V_{L}) comprising a sequence set forth in SEQ ID NO: 140 or the complementarity determining regions (CDRs) thereof and a heavy chain variable region (V_{H}) comprising a sequence set forth in SEQ ID NO: 144 or the CDRs thereof;
(ii) a V_{L} comprising a sequence set forth in SEQ ID NO: 148 or the CDRs thereof and a V_{H} comprising a sequence set forth in SEQ ID NO: 152 or the CDRs thereof;
(iii) a V_{L} comprising a sequence set forth in SEQ ID NO: 156 or the CDRs thereof and a V_{H} comprising a sequence set forth in SEQ ID NO: 160 or the CDRs thereof.

80. (canceled)

81. (canceled)

82. (canceled)

83. A method of preventing, treating, delaying the progression of,
preventing a relapse of, or alleviating a symptom of an autoimmune disease, transplantation rejection, -graft versus host disease, or graft versus tumour effect, a cancer or an infection the method comprising administering the BTN2A1 agonist of claim 77 to a subject in need thereof in an amount sufficient to prevent, treat, delay the progression of, prevent a relapse of, or alleviate the symptom of the autoimmune disease, transplant rejection, graft versus host disease, or graft versus tumour effect in the subject.

84. (canceled)
